SEQUENCE LISTING (75 sequences)

SEQ ID NO: 1 - DNA, 26 bp, Mus musculus
cagcactcagactacgtgcacctctg

SEQ ID NO: 2 - protein, 72 aa, Artificial Sequence
  source: /note=“Description of Artificial Sequence Synthetic polypeptide”
QQQQQQQQQQQQQQQQQQQQQQQQQQQQQQQQQQQQQQQQQQQQQQQQQQQQQQQQQQQQQQQQQQQQQQQQ

SEQ ID NO: 3 - protein, 11 aa, Artificial Sequence
  source: /note=“Description of Artificial Sequence Synthetic peptide”
QQQQQQQQQQQ

SEQ ID NO: 4 - DNA, 15 bp, Mus musculus
tggatgacgtgtaca

SEQ ID NO: 5 - DNA, 28 bp, Mus musculus
tcgagacaggtgctgacgtggcgattcc

SEQ ID NO: 6 - DNA, 21 bp, Artificial Sequence
  source: /note=“Description of Artificial Sequence Synthetic oligonucleotide”
gggattcatgaatggccctta

SEQ ID NO: 7 - DNA, 21 bp, Artificial Sequence
  source: /note=“Description of Combined DNA/RNA Molecule Synthetic oligonucleotide”
guuggacccugucauguuutt

SEQ ID NO: 8 - DNA, 21 bp, Artificial Sequence
  source: /note=“Description of Combined DNA/RNA Molecule Synthetic oligonucleotide”
aaacaugacaggguccaactt

SEQ ID NO: 9 - DNA, 21 bp, Artificial Sequence
  source: /note=“Description of Combined DNA/RNA Molecule Synthetic oligonucleotide”
gccauuaaugaacucauuctt

SEQ ID NO: 10 - DNA, 21 bp, Artificial Sequence
  source: /note=“Description of Combined DNA/RNA Molecule Synthetic oligonucleotide”
gaaugaguucauuaauggctt

SEQ ID NO: 11 - RNA, 21 nt, Artificial Sequence
  source: /note=“Description of Artificial Sequence Synthetic oligonucleotide”
gaagagaaccacaaacuccuu

SEQ ID NO: 12 - RNA, 21 nt, Artificial Sequence
  source: /note=“Description of Artificial Sequence Synthetic oligonucleotide”
ggaguuugugguucucuucuu

SEQ ID NO: 13 - RNA, 21 nt, Artificial Sequence
  source: /note=“Description of Artificial Sequence Synthetic oligonucleotide”
gaggaucacccugaauucauu

SEQ ID NO: 14 - RNA, 21 nt, Artificial Sequence
  source: /note=“Description of Artificial Sequence Synthetic oligonucleotide”
ugaauucagggugauccucuu

SEQ ID NO: 15 - RNA, 21 nt, Artificial Sequence
  source: /note=“Description of Artificial Sequence Synthetic oligonucleotide”
cuuggacccagucauguucuu

SEQ ID NO: 16 - RNA, 21 nt, Artificial Sequence
  source: /note=“Description of Artificial Sequence Synthetic oligonucleotide”
gaacaugacuggguccaaguu

SEQ ID NO: 17 - RNA, 21 nt, Artificial Sequence
  source: /note=“Description of Artificial Sequence Synthetic oligonucleotide”
aucugcuuucauccagccauu

SEQ ID NO: 18 - RNA, 21 nt, Artificial Sequence
  source: /note=“Description of Artificial Sequence Synthetic oligonucleotide”
uggcuggaugaaagcagauuu

SEQ ID NO: 19 - RNA, 21 nt, Artificial Sequence
  source: /note=“Description of Artificial Sequence Synthetic oligonucleotide”
gccccuagucuuagagauauu

SEQ ID NO: 20 - RNA, 21 nt, Artificial Sequence
  source: /note=“Description of Artificial Sequence Synthetic oligonucleotide”
uaucucuaagacuaggggcuu

SEQ ID NO: 21 - RNA, 21 nt, Artificial Sequence
  source: /note=“Description of Artificial Sequence Synthetic oligonucleotide”
gaaccuguagaagaugaccuu

SEQ ID NO: 22 - RNA, 21 nt, Artificial Sequence
  source: /note=“Description of Artificial Sequence Synthetic oligonucleotide”
ggucaucuucuacagguucuu

SEQ ID NO: 23 - RNA, 21 nt, Artificial Sequence
  source: /note=“Description of Artificial Sequence Synthetic oligonucleotide”
guucagauggaauccucuauu

SEQ ID NO: 24 - RNA, 21 nt, Artificial Sequence
  source: /note=“Description of Artificial Sequence Synthetic oligonucleotide”
uagaggauuccaucugaacuu

SEQ ID NO: 25 - RNA, 21 nt, Artificial Sequence
  source: /note=“Description of Artificial Sequence Synthetic oligonucleotide”
uuguguaccggggcauguuuu

SEQ ID NO: 26 - RNA, 21 nt, Artificial Sequence
  source: /note=“Description of Artificial Sequence Synthetic oligonucleotide”
aacaugccccgguacacaauu

SEQ ID NO: 27 - RNA, 21 nt, Artificial Sequence
  source: /note=“Description of Artificial Sequence Synthetic oligonucleotide”
guggggcgcaucaccaaguuu

SEQ ID NO: 28 - RNA, 21 nt, Artificial Sequence
  source: /note=“Description of Artificial Sequence Synthetic oligonucleotide”
acuuggugaugcgccccacuu

SEQ ID NO: 29 - RNA, 21 nt, Artificial Sequence
  source: /note=“Description of Artificial Sequence Synthetic oligonucleotide”
cauucuccucuccaugcccuu

SEQ ID NO: 30 - RNA, 21 nt, Artificial Sequence
  source: /note=“Description of Artificial Sequence Synthetic oligonucleotide”
gggcauggagaggagaauguu

SEQ ID NO: 31 - RNA, 21 nt, Artificial Sequence
  source: /note=“Description of Artificial Sequence Synthetic oligonucleotide”
ugaaggagaaggagacccuuu

SEQ ID NO: 32 - RNA, 21 nt, Artificial Sequence
  source: /note=“Description of Artificial Sequence Synthetic oligonucleotide”
agggucuccuucuccuucauu

SEQ ID NO: 33 - RNA, 21 nt, Artificial Sequence
  source: /note=“Description of Artificial Sequence Synthetic oligonucleotide”
caucucucuuugaacucacuu

SEQ ID NO: 34 - RNA, 21 nt, Artificial Sequence
  source: /note=“Description of Artificial Sequence Synthetic oligonucleotide”
gugaguucaaagagagauguu

SEQ ID NO: 35 - RNA, 21 nt, Artificial Sequence
  source: /note=“Description of Artificial Sequence Synthetic oligonucleotide”
gccugcaaugaaggagaaguu

SEQ ID NO: 36 - RNA, 21 nt, Artificial Sequence
  source: /note=“Description of Artificial Sequence Synthetic oligonucleotide”
cuucuccuucauugcaggcuu

SEQ ID NO: 37 - RNA, 21 nt, Artificial Sequence
  source: /note=“Description of Artificial Sequence Synthetic oligonucleotide”
ccacugcuacagacagcucuu

SEQ ID NO: 38 - RNA, 21 nt, Artificial Sequence
  source: /note=“Description of Artificial Sequence Synthetic oligonucleotide”
gagcugucuguagcagugguu

SEQ ID NO: 39 - DNA, 19 bp, Artificial Sequence
  source: /note=“Description of Artificial Sequence Synthetic primer”
tcatcggacgcacttggaa

SEQ ID NO: 40 - DNA, 21 bp, Artificial Sequence
  source: /note=“Description of Artificial Sequence Synthetic primer”
caaccaccttgaatggcaaga

SEQ ID NO: 41 - DNA, 20 bp, Artificial Sequence
  source: /note=“Description of Artificial Sequence Synthetic primer”
gaggcttgcccctgagtatg

SEQ ID NO: 42 - DNA, 19 bp, Artificial Sequence
  source: /note=“Description of Artificial Sequence Synthetic primer”
gttggcagtgcaatccacc

SEQ ID NO: 43 - DNA, 21 bp, Artificial Sequence
  source: /note=“Description of Artificial Sequence Synthetic primer”
gtccctagcttggctgacaga

SEQ ID NO: 44 - DNA, 18 bp, Artificial Sequence
  source: /note=“Description of Artificial Sequence Synthetic primer”
tggagagcgagggctttg

SEQ ID NO: 45 - DNA, 18 bp, Artificial Sequence
  source: /note=“Description of Artificial Sequence Synthetic primer”
cctgagcccggaggagaa

SEQ ID NO: 46 - DNA, 18 bp, Artificial Sequence
  source: /note=“Description of Artificial Sequence Synthetic primer”
ctcgagcagtctgcgctg

SEQ ID NO: 47 - DNA, 21 bp, Artificial Sequence
  source: /note=“Description of Artificial Sequence Synthetic primer”
caagatcaagccccacctgat

SEQ ID NO: 48 - DNA, 21 bp, Artificial Sequence
  source: /note=“Description of Artificial Sequence Synthetic primer”
agttcgccccaaccagtactt

SEQ ID NO: 49 - DNA, 22 bp, Artificial Sequence
  source: /note=“Description of Artificial Sequence Synthetic primer”
ccccagtgtcaaactgtaccag

SEQ ID NO: 50 - DNA, 24 bp, Artificial Sequence
  source: /note=“Description of Artificial Sequence Synthetic primer”
agcgtttccaattttccataaatt

SEQ ID NO: 51 - DNA, 19 bp, Artificial Sequence
  source: /note=“Description of Artificial Sequence Synthetic primer”
aagccctctggaacttgcg

SEQ ID NO: 52 - DNA, 19 bp, Artificial Sequence
  source: /note=“Description of Artificial Sequence Synthetic primer”
aacccaatggcctgtctgg

SEQ ID NO: 53 - DNA, 21 bp, Artificial Sequence
  source: /note=“Description of Artificial Sequence Synthetic primer”
ctatttccagggcttccgagt

SEQ ID NO: 54 - DNA, 21 bp, Artificial Sequence
  source: /note=“Description of Artificial Sequence Synthetic primer”
aggtgttgtactggcctcggt

SEQ ID NO: 55 - DNA, 20 bp, Artificial Sequence
  source: /note=“Description of Artificial Sequence Synthetic primer”
catgtacctgcaccacgtcg

SEQ ID NO: 56 - DNA, 19 bp, Artificial Sequence
  source: /note=“Description of Artificial Sequence Synthetic primer”
gaggaccaccatcatccgg

SEQ ID NO: 57 - DNA, 20 bp, Artificial Sequence
  source: /note=“Description of Artificial Sequence Synthetic primer”
taccaccatgtacccaggca

SEQ ID NO: 58 - DNA, 24 bp, Artificial Sequence
  source: /note=“Description of Artificial Sequence Synthetic primer”
ctcaggaggagcaatgatcttgat

SEQ ID NO: 59 - DNA, 20 bp, Artificial Sequence
  source: /note=“Description of Artificial Sequence Synthetic primer”
ccatcaacatgctcccgttc

SEQ ID NO: 60 - DNA, 16 bp, Artificial Sequence
  source: /note=“Description of Artificial Sequence Synthetic primer”
gggtaggcctcgccat

SEQ ID NO: 61 - DNA, 21 bp, Artificial Sequence
  source: /note=“Description of Artificial Sequence Synthetic primer”
tgtatgtacgccgcgtattca

SEQ ID NO: 62 - DNA, 21 bp, Artificial Sequence
  source: /note=“Description of Artificial Sequence Synthetic primer”
tcggaatccacaacacctttg

SEQ ID NO: 63 - DNA, 20 bp, Artificial Sequence
  source: /note=“Description of Artificial Sequence Synthetic primer”
acacgcttgggaatggacac

SEQ ID NO: 64 - DNA, 20 bp, Artificial Sequence
  source: /note=“Description of Artificial Sequence Synthetic primer”
ccatgggaagatgttctggg

SEQ ID NO: 65 - DNA, 24 bp, Artificial Sequence
  source: /note=“Description of Artificial Sequence Synthetic primer”
aaacagagtagcagcgcagactgc

SEQ ID NO: 66 - DNA, 26 bp, Artificial Sequence
  source: /note=“Description of Artificial Sequence Synthetic primer”
ggatctctaaaactagaggcttggtg

SEQ ID NO: 67 - DNA, 21 bp, Mus musculus
ccattaatgaactcattcgtt

SEQ ID NO: 68 - DNA, 21 bp, Mus musculus
gctcgtgaattgatagagaaa

SEQ ID NO: 69 - DNA, 21 bp, Mus musculus
gcgggagtatagtgagtttaa

SEQ ID NO: 70 - DNA, 21 bp, Mus musculus
ccattaatgaactcattcgtt

SEQ ID NO: 71 - DNA, 21 bp, Mus musculus
gctcgtgaattgatagagaaa

SEQ ID NO: 72 - DNA, 21 bp, Mus musculus
gcgggagtatagtgagtttaa

SEQ ID NO: 73 - DNA, 21 bp, Mus musculus
ccatatcatatctgtggacaa

SEQ ID NO: 74 - DNA, 21 bp, Mus musculus
gcccttaaagagcatctacat

SEQ ID NO: 75 - protein, 32 aa, Artificial Sequence
  source: /note=“Description of Artificial Sequence Synthetic peptide”
QQQQQQQQQQQQQQQQQQQQQQQQQQQQQQQQ

(19) United States
(12) Patent Application Publication
(10) Pub. No.: US 2011/0142799 A1
(43) Pub. Date: Jun. 16, 2011

(54) MODULATION OF NEURODEGENERATIVE DISEASE BY MODULATING XBP-1 ACTIVITY

(75) Inventors: Laurie H. Glimcher, West Newton, MA (US); Claudio Hetz, Brookline, MA (US)

(73) Assignee: PRESIDENT AND FELLOWS OF HARVARD COLLEGE, Cambridge, MA (US)

(21) Appl. No.: 13/000,921

(22) PCT Filed: Jun. 23, 2009
(86) PCT No.: PCT/US09/48321

§ 371 Date: Mar. 2, 2011

Related U.S. Application Data
(60) Provisional application No. 61/132,836, filed on Jun. 23, 2008.

(51) Int. Cl. A61K 38/21; C40B 30/06; C40B 30/04; C12N 5/0793; A61K 31/7016; A61K 31/437; A61K 31/138; A61K 31/436; A61K 31/131; A61K 31/683; A61K 31/198; A61K 31/575; A61K 31/192; A61K 31/10; A61K 31/047; A61P 25/00; C12Q 1/68

(52) U.S. Cl. 424/85.5; 506/10; 506/9; 435/375; 514/53; 514/283; 514/648; 514/291; 514/645; 514/663; 514/114; 514/561; 514/182; 514/570; 514/708; 514/738; 435/6.1

(57) ABSTRACT

The invention provides methods for treating and preventing a neurodegenerative disease associated with aberrant protein aggregation by modulating the expression and/or activity of XBP-1, IRE-1 alpha, and/or EDEM. The present invention also pertains to methods for identifying compounds that modulate the expression and/or activity of XBP-1, 1RE-1 alpha, and/or EDEM. The present invention further provides methods for determining whether a subject is at risk of developing or has developed a neurodegenerative disease associated with aberrant protein aggregation.

### RELATED APPLICATIONS

This application claims the benefit of Provisional Application U.S. Ser. No. 61/132,836 titled “Modulation of neurodegenerative disease by modulating XBP-1 activity” filed on Jun. 23, 2008.

This application is related to U.S. patent application Ser. No. 10/655,620, filed Sep. 2, 2003, titled “Methods and Compositions for Modulating XBP-1 Activity”, which claims priority to U.S. Provisional Application Ser. No. 60/407,166, titled “Methods and Compositions for Modulating XBP-1 Activity”, filed Aug. 30, 2002, and U.S. Provisional Application Ser. No. 60/488,568 titled “Methods and Compositions for Modulating XBP-1 Activity”, filed Jul. 18, 2003.

This application is also related to U.S. Provisional Patent Application No. 61/011,070, filed Jan. 14, 2008, titled “Methods for Moddulating De Novo Hepatic Lipogenesis by Modulating XBP-1 Activity”.

The entire contents of each of these applications are incorporated herein by this reference.

### BACKGROUND OF THE INVENTION

Amyotrophic lateral sclerosis (ALS), also known as Lou Gehrig's disease and Maladie de Charcot, is a progressive and deadly adult-onset motor neuron disease characterized by muscle weakness, spasticity, atrophy, paralysis and premature death. The pathological hallmark of ALS is the selective degeneration of motoneurons in the spinal ventral horn, most brainstem nuclei and the cerebral cortex. ALS has an average age of onset of approximately 50 years and the annual incidence is estimated at 1-2 per 100,000 individuals with a higher frequency in males than females. The majority of ALS patients lacks a defined hereditary genetic component and is termed sporadic, while approximately 10% of cases are familial (fALS). About 20 percent of all familial cases result from a specific genetic defect that leads to mutation of the enzyme known as superoxide dismutase 1 (SOD1). Over 100 mutations affect the SOD1 gene have been identified and shown to trigger misfolding and abnormal aggregation of the SOD1 protein resulting in neurotoxicity. Overexpression of human fALS-linked SOD1 mutations in transgenic mice recapitulates essential features of the human pathology, provoking age-dependent protein aggregation, paralysis and motor neuron degeneration (see general reviews in Pasinelli, P. and Brown, R. H. (2006). Nat. Rev. Neurosci. 7, 710-723; Boillee, S., et al. (2006). Neuron 52, 39-59). Since sporadic and familial ALS affect the same neurons with similar pathology, therapeutics effective in mutant SOD1 models may translate to sporadic ALS.

The primary mechanism by which mutations in SOD1 contribute to progressive motoneuron loss in fALS remains unknown, but it has been proposed that neuronal loss is mediated by different mechanisms including mitochondrial dysfunction, altered axonal transport, and other important non-neuronal components (reviewed in Pasinelli, P. and Brown, R. H. (2006). Nat. Rev. Neurosci. 7, 710-723; Boillee, S., et al. (2006). Neuron 52, 39-59). Autophagy is a “large-scale” cellular degradation process for proteins and damaged organelles (Maiuri et al., 2007. Nat Rev Mol Cell Biol 8:741). Macroautophagy, a process generally referred to as autophagy, involves the formation of double-membrane-bound structures known as autophagosomes. These fuse with lysosomes to form autophagolysosomes or autolysosomes, whose contents are then degraded by acidic lysosomal hydrolases.

Autophagy is also critical for the maintenance of neuronal homoeostasis (Cuervo, 2006. Trends Mol Med 12:461), and was recently shown to be an efficient pathway to eliminate mutant proteins related to neurodegeneration (review in Rubinzstein 2006 Nature 443:780; Matus et al. 2008 Curr Mol Med 8:157). Moreover, analysis of sporadic and familial ALS post-mortem samples revealed a strong activation of the UPR and autophagy.

Recent data also suggest that stress responses originating from the endoplasmic reticulum (ER) may contribute to ALS (Turner, B. J. and Atkin, J. D. (2006). Curr. Mol. Med. 6, 79-86). The ER can be thought of as a sophisticated machine for protein folding and secretion that employs an efficient system of chaperones to promote folding and prevent abnormal aggregation of proteins. A number of stress conditions can interfere with the function of this organelle, leading to abnormal protein folding in the ER lumen, resulting in a cellular condition referred to as ER stress. To alleviate ER stress, cells activate an integrated signaling pathway known as the Unfolded Protein Response (UPR), which aims to re-establish homeostasis by decreasing the extent of protein misfolding (review in Schroder, M. and Kaufman, R. J. (2005). Annu. Rev. Biochem. 74, 739-789). Disturbances in ER function are thought to contribute to cell loss in a number of important human diseases including Parkinson's (Hoozemans, J. J., et al. (2007). Biochem. Biophys. Res. Commun. 354, 707-711), Alzheimer's disease (Unterberger, U., et al. (2006). J. Neuropathol. Exp. Neurol. 65, 348-357), and many others (review in Hetz, C. A. and Soto, C. (2006). Curr. Mol. Med. 6, 37-43; Rao, R. V. and Bredesen, D. E. (2004). Curr. Opin. Cell Biol. 16, 653-662).

ER stress responses are observed in transgenic rodents expressing different fALS-related SOD1 mutations (Vlug, A. S., et al. (2005). Eur. J. Neurosci. 22, 1881-1894; Nagata, T., et al. (2007). Neurol. Res. 29, 767-771; Kikuchi, H., et al. (2006). Proc. Natl. Acad. Sci. U.S.A 103, 6025-6030; Wootz, H., et al. (2004). Biochem. Biophys. Res. Commun. 322, 281-286; Atkin, J. D., et al. (2006). J. Biol. Chem. 281, 30152; Urushitani, M., et al. (2006). Nat. Neurosci. 9, 108; Kieran, D., et al. (2007). Proc. Natl. Acad. Sci. U.S.A 104, 20606) and biochemical and histological studies revealed that a fraction of mutant SOD1 accumulates inside the ER and Golgi in vivo, where it forms insoluble high molecular weight species (Atkin, J. D., et al. (2006). J. Biol. Chem. 281, 30152-30165; Kikuchi, H., et al. (2006). Proc. Natl. Acad. Sci. U.S.A 103, 6025; Urushitani, M., et al. (2006). Nat. Neurosci. 9, 108). A direct interaction between mutant SOD1 and the ER chaperones Grp78/BiP and PDI was observed in microsomal fractions of spinal cord extracts. Significantly, a proteomic analysis of spinal cord tissue from SOD1^{G93A} transgenic mice revealed that two UPR targets, PDI and Grp58, are among the most induced proteins in this ALS model. Moreover, the involvement of the pro-apoptotic genes BIM and PUMA in motoneuron loss and disease progression in fALS models were recently described (Hetz et al., 2007; Kieran, D., et al. (2007). Proc. Natl. Acad. Sci. U.S.A 104, 20606), both genes essential for ER stress-induced apoptosis (Morishima, N., et al. (2004). J. Biol. Chem. 279, 50375; Puthalakath, H., et al. (2007). Cell 129, 1337).

Further elucidation of the molecular mechanisms involved in neurodegenerative diseases associated with protein aggregation would be of considerable benefit in identifying targets for drug discovery and in providing methods for treating, preventing, and/or delaying the onset neurodegenerative diseases associated with protein aggregation.

### SUMMARY OF THE INVENTION

The instant invention is based, at least in part, on the discovery that XBP-1 deficieny protects against neurodegenerative disease associated with protein aggregation. In particular, although it has previously been observed that XBP-1 deficiency results in failure to handle ER stress in organs such as pancreas and B lymphocytes, leading to apoptosis, the inventors have surprisingly discovered that XBP-1 deficiency in motor neurons in an animal model of neurodegenerative disease associated with protein aggregation, increases the life span of the animals and reduces motor neuron apoptosis.

Furthermore, the present invention is based, at least in part, on the discovery that reduced expression of XBP-1, IRE-1 alpha, and EDEM (e.g., using shRNAs) in in vitro models of neurodegenerative disease associated with protein aggesgation, e.g., ALS and Huntington's disease (HD), decreases the accumulation of intracellular inclusions of protein aggregates associated with the neurodegenerative disease, increases cell survival (decreases cell, e.g., motor neuron cell, apoptosis), and increases the levels of autophagy in the cells, e.g., motor neuron cells.

Accordingly, in one aspect, the invention pertains to methods of identifying compounds useful in treating a neurodegenerative disease associated with aberrant protein aggregation comprising, a) providing an indicator cell comprising an XBP-1 polypeptide; b) contacting the indicator cell with each member of a library of test compounds; c) determining the activity of the XBP-1 polypeptide in the presence and absence of the test compound; d) selecting from the library of test compounds a compound of interest that downmodulates the activity of the XBP-1 polypeptide; e) determining the effect of the compound on autophagy; and f) selecting from the library of test compounds a compound of interest that increases autophagy as compared to an appropriate control, thereby identifying a compound useful in treating a neurodegenerative disease associated with aberrant protein aggregation.

In another aspect, the invention pertains to a method of identifying compounds useful in preventing a neurodegenerative disease associated with aberrant protein aggregation comprising, a) providing an indicator cell comprising an XBP-1 polypeptide; b) contacting the indicator cell with each member of a library of test compounds; c) determining the activity of the XBP-1 polypeptide in the presence and absence of the test compound; d) selecting from the library of test compounds a compound of interest that downmodulates the activity of the XBP-1 polypeptide; e) determining the effect of the compound on autophagy; and f) selecting from the library of test compounds a compound of interest that increases autophagy as compared to an appropriate control, thereby identifying a compound useful in preventing a neurodegenerative disease associated with aberrant protein aggregation.

In another aspect, the invention pertains to a method of identifying compounds useful in delaying the onset of a neurodegenerative disease associated with aberrant protein aggregation comprising, a) providing an indicator cell comprising an XBP-1 polypeptide; b) contacting the indicator cell with each member of a library of test compounds; c) determining the activity of the XBP-1 polypeptide in the presence and absence of the test compound; d) selecting from the library of test compounds a compound of interest that downmodulates the activity of the XBP-1 polypeptide; e) determining the effect of the compound on autophagy; and

f) selecting from the library of test compounds a compound of interest that increases autophagy as compared to an appropriate control, thereby identifying a compound useful in delaying the onset of a neurodegenerative disease associated with aberrant protein aggregation.

In another aspect, the invention pertains to a method of identifying compounds useful in treating a neurodegenerative disease associated with aberrant protein aggregation comprising,

a) providing an indicator cell comprising an IRE-1 alpha polypeptide;

b) contacting the indicator cell with each member of a library of test compounds;

c) determining the activity of the IRE-1 alpha polypeptide in the presence and absence of the test compound;

d) selecting from the library of test compounds a compound of interest that downmodulates the activity of the IRE-1 alpha polypeptide;

e) determining the effect of the compound on autophagy; and

f) selecting from the library of test compounds a compound of interest that increases autophagy as compared to an appropriate control, thereby identifying a compound useful in treating a neurodegenerative disease associated with aberrant protein aggregation.

In another aspect, the invention pertains to a method of identifying compounds useful in preventing a neurodegenerative disease associated with aberrant protein aggregation comprising,

a) providing an indicator cell comprising an IRE-1 alpha polypeptide;

b) contacting the indicator cell with each member of a library of test compounds;

c) determining the activity of the IRE-1 alpha polypeptide in the presence and absence of the test compound;

d) selecting from the library of test compounds a compound of interest that downmodulates the activity of the IRE-1 alpha polypeptide;

e) determining the effect of the compound on autophagy; and

f) selecting from the library of test compounds a compound of interest that increases autophagy as compared to an appropriate control, thereby identifying a compound useful in preventing a neurodegenerative disease associated with aberrant protein aggregation.

In another aspect, the invention pertains to a method of identifying compounds useful in delaying the onset of a neurodegenerative disease associated with aberrant protein aggregation comprising,

a) providing an indicator cell comprising an IRE-1 alpha polypeptide;

b) contacting the indicator cell with each member of a library of test compounds;

c) determining the activity of the IRE-1 alpha polypeptide in the presence and absence of the test compound;

d) selecting from the library of test compounds a compound of interest that downmodulates the activity of the IRE-1 alpha polypeptide;

e) determining the effect of the compound on autophagy; and

f) selecting from the library of test compounds a compound of interest that increases autophagy as compared to an appropriate control, thereby identifying a compound useful in delaying the onset of a neurodegenerative disease associated with aberrant protein aggregation.

In another aspect, the invention pertains to a method of identifying compounds useful in treating a neurodegenerative disease associated with aberrant protein aggregation comprising,

a) providing an indicator cell comprising an EDEM polypeptide;

b) contacting the indicator cell with each member of a library of test compounds;

c) determining the activity of the EDEM polypeptide in the presence and absence of the test compound;

d) selecting from the library of test compounds a compound of interest that downmodulates the activity of the EDEM polypeptide;

e) determining the effect of the compound on autophagy; and

f) selecting from the library of test compounds a compound of interest that increases autophagy as compared to an appropriate control, thereby identifying a compound useful in treating a neurodegenerative disease associated with aberrant protein aggregation.

In still another aspect, the invention pertains to a method of identifying compounds useful in preventing a neurodegenerative disease associated with aberrant protein aggregation comprising,

a) providing an indicator cell comprising an EDEM polypeptide;

b) contacting the indicator cell with each member of a library of test compounds;

c) determining the activity of the EDEM polypeptide in the presence and absence of the test compound;

d) selecting from the library of test compounds a compound of interest that downmodulates the activity of the EDEM polypeptide;

e) determining the effect of the compound on autophagy; and

f) selecting from the library of test compounds a compound of interest that increases autophagy as compared to an appropriate control, thereby identifying a compound useful in preventing a neurodegenerative disease associated with aberrant protein aggregation.

In still another aspect, the invention pertains to a method of identifying compounds useful in delaying the onset of a neurodegenerative disease associated with aberrant protein aggregation comprising,

a a) providing an indicator cell comprising an EDEM polypeptide;

b) contacting the indicator cell with each member of a library of test compounds;

c) determining the activity of the EDEM polypeptide in the presence and absence of the test compound;

d) selecting from the library of test compounds a compound of interest that downmodulates the activity of the EDEM polypeptide;

e) determining the effect of the compound on autophagy; and

f) selecting from the library of test compounds a compound of interest that increases autophagy as compared to an appropriate control, thereby identifying a compound useful in delaying the onset of a neurodegenerative disease associated with aberrant protein aggregation.

In one embodiment, the activity of the XBP-1, IRE-1 alpha, or EDEM polypeptide is determined by determining the level of expression of the mRNA encoding the XBP-1, IRE-1 alpha, or EDEM polypeptide.

In one embodiment, the activity of the XBP-1, IRE-1 alpha, or EDEM polypeptide is determined by determining the level of XBP-1, IRE-1 alpha, or EDEM protein levels.

In one embodiment, the effect of the compound on XBP-1 or IRE-1 alpha activity is determined by measuring the binding of XBP-1 to IRE-1.

In one embodiment, the effect of the compound on XBP-1 activity is determined by assaying the activity of IRE-1.

In one embodiment, the activity of IRE-1 is a kinase activity.

In one embodiment, the activity of IRE-1 is an endoribonuclease activity.

In one embodiment, the XBP-1 polypeptide comprises a transactivation domain and the indicator composition further comprises a vector comprising a regulatory element responsive to the XBP-1 transactivation domain operatively linked to a reporter gene and the effect of the compound on the activity of the polypeptide is determined by evaluating the expression of the reporter gene in the presence and absence of the test compound.

In one embodiment, the indicator composition comprises a recombinant expression vector encoding a spliced XBP-1 protein; and a vector comprising a regulatory element responsive to the XBP-1 protein operatively linked a reporter gene and the effect of the compound on the activity of the polypeptide is determined by evaluating the expression of the reporter gene in the presence and absence of the test compound.

In one embodiment, the indicator composition is a cell and comprises, a recombinant expression vector comprising a regulatory element responsive to XBP-1 spliced protein operatively linked a reporter gene, wherein the cell is contacted with an agent that induces ER stress, and the effect of the compound on the activity of the polypeptide is determined by evaluating the expression of the reporter gene in the presence and absence of the test compound.

In one embodiment, the method further comprises determining the effect of the identified compound on the ratio of unspliced XBP-1 to spliced XBP-1 mRNA and/or protein.

In one embodiment, the method further comprises determining the effect of the identified compound on the production of XBP-1 protein.

In one embodiment, the activity of the EDEM polypeptide is determined by determining the binding of EDEM to calnexin.

In one embodiment, the activity of the EDEM polypeptide is determined by determining the ubiquitinated cellular protein levels

In one embodiment, the effect of the compound on autophagy is determined by determining the effect on the compound on motor neuron survival.

In one embodiment, the effect of the compound on autophagy is determined by determining the effect on the compound on generation of protein aggregation associated with the neurodegenerative disease.

In one embodiment, the effect of the compound on autophagy is determined by determining the effect on the compound on apoptosis of the cell.

In one embodiment, the cell further comprises an expression vector comprising a nucleic acid molecule encoding a SOD1 polypeptide, and the effect of the compound on autophagy is determined by determining the effect on the compound on intracellular accumulation of SOD1 inclusions and/or the detergent insolubility of SOD1.

In one embodiment, the SOD1 polypeptide comprises SOD^{G93A} or SOD^{G86R}.

In one embodiment, the expression vector further comprises a nucleic acid molecule encoding a heterologous polypeptide.

In one embodiment, the indicator cell further comprises an expression vector comprising a nucleic acid molecule encoding an LC3 polypeptide.

In one embodiment, the expression vector further comprises a nucleic acid molecule encoding a heterologous polypeptide.

In one embodiment, the heterologous polypeptide comprises dsRED. In one embodiment, the heterologous polypeptide comprises EGFP.

In one embodiment, the indicator cell further comprises an expression vector comprising a nucleic acid molecule encoding polyglutamine, and the effect of the compound on autophagy is determined by determining the effect on the compound on intracellular accumulation of polyglutamine inclusions and/or the detergent insolubility of polyglutamine.

In one embodiment, the cell has been engineered to express the polypeptide by introducing into the cell an expression vector encoding the polypeptide.

In one embodiment, the indicator cell is a motor neuron cell.

In one embodiment, the indicator cell is contacted with an agent that induces ER stress. In one embodiment, the agent is tunicamycin or thapsigargin.

In one embodiment, the indicator cell is contacted with an inhibitor of autophagy.

In one embodiment, the inhibitor is 3-methyladenine or Wortmannin.

In one embodiment, the indicator cell is undergoing nutrient starvation.

In one embodiment, the method further comprises determining the effect of the compound on the number of autophagosomes.

In one embodiment, the method further comprises determining the effect of the compound on lysosome content.

In one embodiment, the method further comprises determining the effect of the compound on Beclin-1 expression.

In one embodiment, the method further comprises determining the effect of the compound on expression and/or processing of LC3.

In one embodiment, the method further comprises determining the expression of a gene responsive to the activity of the polypeptide.

In one embodiment, the gene is selected from the group consisting of XBP-1, IRE-1 alpha, EDEM, ERdj4, Gpr58, Sec61, Wfs-1, Herp, Chop.

In one embodiment, the method further comprises determining the splicing of XBP-1.

In one embodiment, the method further comprises determining IRE-1 kinase activity.

In one embodiment, the method further comprises, further comprising determining the effect of the identified compound on the effect of the compound on a neurodegenerative disease associated with protein aggregation in a non-human animal, comprising administering the test compound to the animal and determining the effect of test compound on disease onset, disease progression, and/or disease severity in the presence and absence of the test compound, to thereby identify a compound that modulates a neurodegenerative disease associated with protein aggregation.

In one embodiment, the non-human animal develops Amyotrophic lateral sclerosis or a similar disorder.

In one embodiment, the animal comprises a SOD1 transgene.

In one embodiment, the non-human animal develops Huntington's disease or a similar disorder.

In one embodiment, the animal comprises an HD transgene.

In one embodiment, the neurodegenerative disease associated with aberrant protein aggregation is Amyotrophic lateral sclerosis.

In one embodiment, the Amyotrophic lateral sclerosis is familial Amyotrophic lateral sclerosis.

In one embodiment, the aggregated protein is a SOD1 protein.

In one embodiment, the Amyotrophic lateral sclerosis is sporadic Amyotrophic lateral sclerosis.

In one embodiment, the neurodegenerative disease associated with protein aggregation is Huntington's disease.

In one embodiment, the compound is present in a small molecule microarray.

In one aspect, the invention pertains to a method for decreasing protein aggregation in a motor neuron cell, comprising contacting the cell with an agent that decreases the activity of XBP-1, thereby decreasing protein aggregation in a motor neuron cell.

In one aspect, the invention pertains to a method for decreasing apoptosis of a motor neuron cell, comprising contacting the cell with an agent that decreases the activity of XBP-1, thereby decreasing apoptosis of a motor neuron cell.

In one aspect, the invention pertains to a method for decreasing protein aggregation in a motor neuron cell, comprising contacting the cell with an agent that decreases the activity of IRE-1 alpha, thereby decreasing protein aggregation in a motor neuron cell.

In one aspect, the invention pertains to a method for decreasing apoptosis of a motor neuron cell, comprising contacting the cell with an agent that decreases the activity of IRE-1 alpha, thereby decreasing apoptosis of a motor neuron cell.

In one aspect, the invention pertains to a method for decreasing protein aggregation in a motor neuron cell, comprising contacting the cell with an agent that decreases the activity of EDEM, thereby decreasing protein aggregation in a motor neuron cell.

In one aspect, the invention pertains to a method for decreasing apoptosis of a motor neuron cell, comprising contacting the cell with an agent that decreases the activity of EDEM, thereby decreasing apoptosis of a motor neuron cell.

In one aspect, the invention pertains to a method for treating a subject with a neurodegenerative disease associated with protein aggregation, comprising administering to the subject an agent that decreases the activity of XBP-1, thereby treating a neurodegenerative disease associated with protein aggregation in the subject.

In one aspect, the invention pertains to a method for preventing a neurodegenerative disease associated with protein aggregation in a subject, comprising administering to the subject an agent that decreases the activity of XBP-1, thereby preventing a neurodegenerative disease associated with protein aggregation in the subject.

In one aspect, the invention pertains to a method for delaying the onset of a neurodegenerative disease associated with protein aggregation in a subject, comprising administering to the subject an agent that decreases the activity of XBP-1, thereby delaying the onset of the neurodegenerative disease associated with protein aggregation in the subject.

In one aspect, the invention pertains to a method for treating a subject with a neurodegenerative disease associated with protein aggregation, comprising administering to the subject an agent that decreases the activity of IRE-1 alpha, thereby treating a neurodegenerative disease associated with protein aggregation in the subject.

In one aspect, the invention pertains to a method for preventing a neurodegenerative disease associated with protein aggregation in a subject, comprising administering to the subject an agent that decreases the activity of IRE-1 alpha, thereby preventing a neurodegenerative disease associated with protein aggregation in the subject.

In one aspect, the invention pertains to a method for delaying the onset of a neurodegenerative disease associated with protein aggregation in a subject, comprising administering to the subject an agent that decreases the activity of IRE-1 alpha, thereby delaying the onset of the neurodegenerative disease associated with protein aggregation in the subject.

In one aspect, the invention pertains to a method for treating a subject with a neurodegenerative disease associated with protein aggregation, comprising administering to the subject an agent that decreases the activity of EDEM, thereby treating a neurodegenerative disease associated with protein aggregation in the subject.

In one aspect, the invention pertains to a method for preventing a neurodegenerative disease associated with protein aggregation in a subject, comprising administering to the subject an agent that decreases the activity of EDEM, thereby preventing a neurodegenerative disease associated with protein aggregation in the subject.

In one aspect, the invention pertains to a method for delaying the onset of a neurodegenerative disease associated with protein aggregation in a subject, comprising administering to the subject an agent that decreases the activity of EDEM, thereby delaying the onset of the neurodegenerative disease associated with protein aggregation in the subject.

In one embodiment, the neurodegenerative disease associated with protein aggregation is Amyotrophic lateral sclerosis.

In one embodiment, the Amyotrophic lateral sclerosis is familial Amyotrophic lateral sclerosis.

In one embodiment, the Amyotrophic lateral sclerosis is sporadic Amyotrophic lateral sclerosis.

In one embodiment, the neurodegenerative disease associated with protein aggregation is Huntington's disease.

In one embodiment, wherein the agent increases autophagy.

In one embodiment, wherein the agent increases motor neuron survival.

In one embodiment, wherein the agent decreases apoptosis.

In one embodiment, the agent is a chemical chaperone or an autophagy activator.

In one embodiment, the chemical chaperone is selected from the group consisting of glycerol, D20, dimethylsulfoxide (DMSO), 4-phenyl butyrate (PBA), tauroursodeoxycholic acid (TUDCA), ursodeoxycholic acid (UDCA), glycine betaine (betaine), glycerolphosphocholine (GPC), methylamines, and trimethylamine N-oxide (TMAO).

In one embodiment, the autophagy activator is selected from the group consisting of a proteasome inhibitor, rapamycin, analogues, and derivatives thereof, tamoxifen, IFN-gamma, trehalose and vinblastine.

In another aspect, the invention pertains to a method for determining the predisposition of a subject to develop a neurodegenerative disorder associated with aberrant protein aggegation, said method comprising determining the amount of one or more markers selected from the group consisting of XBP-1, ERdj4, EDEM, WFS1, Grp58, and PDI in a biological sample derived from the subject and comparing the amount in said sample to the activity of the marker in an appropriate control sample, wherein an increase in the amount of the marker in the sample relative to the amount of the marker in the control sample indicates that the subject is at risk of developing neurodegenerative disorder associated with aberrant protein aggegation.

In another aspect, the invention pertains to a method for determining whether a subject is afflicted with a neurodegenerative disorder associated with aberrant protein aggegation, said method comprising determining the amount of one or more markers selected from the group consisting of XBP-1, ERdj4, EDEM, WFS1, Grp58, and PDI in a biological sample derived from the subject and comparing the amount in said sample to the amount of the marker in an appropriate control sample, wherein an increase in the amount of the marker in the sample relative to the amount of the marker in the control sample indicates that the subject is afflicted with a neurodegenerative disorder associated with aberrant protein aggregation.

### BRIEF DESCRIPTION OF THE FIGURES

FIGS. 1(A)-(D) are figures depicting the engagement of the UPR in familial and sporadic ALS cases. (A) The level of different UPR markers was investigated in total protein extracts derived from spinal cord post mortem samples from different patients affected with sALS and fALS. Healthy controls were also studied. The levels of XBP-1s, ATF4, phospho-ERK, ER chaperones (Grp94, Grp78/BiP and Grp58) and cytosolic chaperons (Hsp70 and Hsp90) were analyzed by Western blot. (B) The expression levels of the ER stress inducible genes PDI, BiP, and CHOP were analyzed in spinal cord protein extracts from symptomatic hSOD1^{G93A}, mSOD1^{G86R} transgenic or control mice by Western blot. All animals were between 100 to 120 days of age. As controls, the expression levels of Hsp90 and SOD1 are shown. Of note, human SOD1 (hSOD1), and mouse SOD1 (mSOD1) proteins are resolved in the analysis. Each well represents different animals. (C) The levels of XBP-1 mRNA splicing were determined in the spinal cord of a symptomatic SOD1^{G86R} transgenic mice by RT-PCR. The DNA fragments corresponding to spliced and non-spliced XBP-1 mRNA are indicated. Samples from three transgenic mice are shown. (D) The mRNA levels of XBP-1 target genes erdj4 and wfs-1, in addition to bip and chop were analyzed by real time PCR in total cDNA obtained from the spinal cord of from SOD1^{G86R} or litter mate control mice. All samples were normalized with the levels of beta-actin on each sample and represent the average four control mice and five SOD1 transgenic. Indicated p values were calculated using Student's t-test.

FIGS. 2(A)-(C) depict that neuronal specific XBP-1 deficiency prolongs life span of mutant SOD1 transgenic mice. (A) XBP-1^{Nes-/-} and control mice were crossed with a SOD1^{G86R} transgenic mice and animal survival was evaluated. Total number of animals per group is indicated. (B) Life span was evaluated in female animals. (C) TUNEL positive cells in the right haft of ventral horn were quantified in a total of five animals per group in mice around 120 days of age. Indicated p values were calculated using Student's t-test.

FIGS. 3(A)-(G) depict that NSC34 motoneuron-like cells were stably transduced with lentiviral vectors expressing shRNA against the XBP-1 or control luciferase mRNA (shXBP-1 and shControl respectively), and expression of XBP-1s or ATF4 (negative control) was analyzed after treatment with the ER stress inducer tunicamycin (Tm) for indicated time points. As a loading control the levels of Hsp90 were analyzed. (B) In parallel IRE-1alpha mRNA was targeted with shRNA and analyzed as described in (A). XBP-1s levels were analyzed after Tm treatment. In addition, the levels of XBP-1 mRNA splicing were determined by RT-PCR. (C) To address the effects of XBP-1 shRNA targeting in NSC34 cells, the mRNA levels of several UPR-target genes were determined by real time PCR. All samples were normalized with the levels of beta-actin on each sample. Results are representative of three independent experiments performed in duplicates. (D) NSC34 stably expressing shRNA constructs against IRE-1 alpha, XBP-1 or control mRNA were transiently transfected with expression vectors for human SOD1^{WT}- (wild-type), SOD1^{G93A}- and SOD1^{G85R}-EFGP fusion proteins. After 72 h, SOD1 intracellular inclusions were quantified by the visualization of SOD1-EGFP distribution by fluorescent microscopy. Left panel: The number of cells presenting intracellular inclusions was quantified in a total of at least 300 cells per experiment. Results are representative of three independent experiments performed in duplicates. Representative pictures of SOD1 distribution are shown. (E) In parallel, detergent insoluble SOD1 protein aggregates were determined in cell extracts prepared in NP-40 and centrifuged at high speed. NP40 insoluble and soluble SOD1 was analyzed by Western blot using anti-GFP antibody. Of note, high molecular weight SOD1 aggregates are observed in the analysis. shRNA cells: Mock (M), XBP-1 (X) or IRE-1 alpha (I). (F) NSC34 cells were co-transfected with mutant SOD1-EGFP and an XBP-1s expression vector or empty pcDNA.3 vector. After 48 h, intracellular inclusions were observed by fluorescent microscopy. (G) In parallel, SOD1 aggregation was determined by Western blot.

FIGS. 4(A)-(F) depict that XBP-1 deficiency leads to autophagy-mediated degradation of mutant SOD1. (A) NSC34 cells expressing shRNA constructs against IRE-1 alpha and XBP-1 were transfected with an expression vector for SOD1^{G85R} and after 48 h cells were treated for 8 h with MG132 (10 and 1 μM), or 16 h with 10 mM 3-MA or 10 μM wortmannine. Then level of SOD1 aggregation was analyzed by Western blot. A lower exposition of the same Western blot is shown in the bottom panel to depict monomeric SOD1. (B) In parallel, similar experiments as shown in (A) were performed after the expression of poly(Q)₇₂. (C) The Western blot signal corresponding to high molecular weight poly(Q)₇₂ aggregates of figure (B) was quantified using a Versadoc machine and plotted as arbitrary units of band pixel intensity. (D) shXBP-1 NSC34 cells were transiently transduced with lentiviruses carring an shRNA against Beclin-1 mRNA. Then the levels of beclin-1 and SOD1^{G85R} aggregation were determined by Western blot. (E) NSC34 cells were co-transfected with a SOD1^{WT} or SOD1^{G85R}-EFGP construct (green) with a LC3-dsRED expression vector, and after 48 h cells were visualized by confocal microscopy. (F) NSC34 cells were transduced with a SOD1^{WT}-EFGP and SOD1^{G85R}-EFGP construct and after 72 h living cells were stained with lysotracker, then fixed and visualized by confocal microscopy. The nucleus was labeled with Hoechst.

FIGS. 5(A)-(D) demonstrate that XBP-1 deficiency decreases the levels of Huntington's disease-related poly(Q)₇₂ aggregates. (A) As an alternative disease model, we monitored poly(Q) aggregation. NSC34 cells transduced with shRNA constructs were transiently transfected with expression vectors for poly(Q)₁₁ (control) or poly(Q)₇₂ as EFGP fusion proteins. The number of cells presenting intracellular poly(Q)_{72 inclusions was quantified in at least a total of} 150 cells per experiment. Results are representative of three independent experiments performed in duplicates. (B) The levels of poly(Q)₁₁ or poly(Q)₇₂ aggregation was determined in protein extracts prepared in RIPA buffer and analyzed by Western blot using anti-GFP antibody. Of note, high molecular weight poly(Q)₇₂ aggregates are observed in the analysis. Duplicate experiments are shown to illustrate reproducibility of the results. (C) NSC34 cells were co-transfected with mutant poly(Q)₇₂ and an XBP-1s expression vector or empty pcDNA.3 vector. After 48 h, intracellular inclusions were observed by fluorescent microscopy. Low magnification pictures are presented. Poly(Q)₇₂ intracellular inclusions were quantified by the visualization of EGFP distribution by fluorescent microscopy. A total of at least 500 cells were counted per experiment. All results are representative of two experiments performed in duplicates. (D) NSC34 cells were transduced with a poly(Q)₁₁ and poly(Q)₇₂-EFGP fusion proteins and after 72 h living cells were stained with lysotracker, then fixed and visualized by confocal microscopy. Nucleus was labeled with Hoechst. Of note, cells expressing intranuclear poly(Q)₇₂ inclusions shown increased number of lysosomes compared with the non-expressing cells (EGFP negative) (see FIG. 10B).

FIGS. 6(A)-(G) show that XBP-1 deficiency increases basal autophagy. (A)

Number of cells presenting autophagosomes was quantified in shControl or shXBP-1 NSC34 cells expressing a LC3-EGFP expression vector in images obtained with a confocal microscope. Quantifications were divided in two subgroups, cells showing 3-4 LC3-EGFP dots or cells containing >5 dots. As control, cells were treated for 16 h with 10 mM 3-MA to inhibit autophagy. Right panel: Representative images are presented to depict autophagosome formation. (B) NSC34 cells were stably transduced with lentiviral vectors expressing shRNA against the XBP-1 or luciferase mRNA (shXBP-1 and shControl respectively), and number of lysosomes (white arrows) were visualized after lysotracker staining and confocal microscopy analysis. The nucleus was stained with Hoechst. (C) NSC34 cells carrying indicated shRNA constructs were stained with acridine orange and then quantified by FACS. Upper panel: Representative images of the staining are shown. As positive control, cells were starved in EBSS buffer for 1 h. Representative confocal images of acridine orange staining are also shown. (D) Autophagosomes were visualized in shControl and shXBP-1 NSC34 cells by electron microscopy. Multimembrane autophagosomes are indicated with an arrow. (E) shControl, shXBP-1 and shIRE-1 alpha cells were maintained in rich culture media or incubated in EBSS buffer for 2 h. Then cell viability was determined after propidium iodide (PI) staining. Overlap images of phase contrast and PI staining are shown. Results are representative of three independent experiments performed in duplicates. (F) Cell viability was measured using the MTS assay using the conditions described in E. (G) XBP-1 mRNA splicing was evaluated in experiments presented in (E) in shLuc or shIRE1 cells.

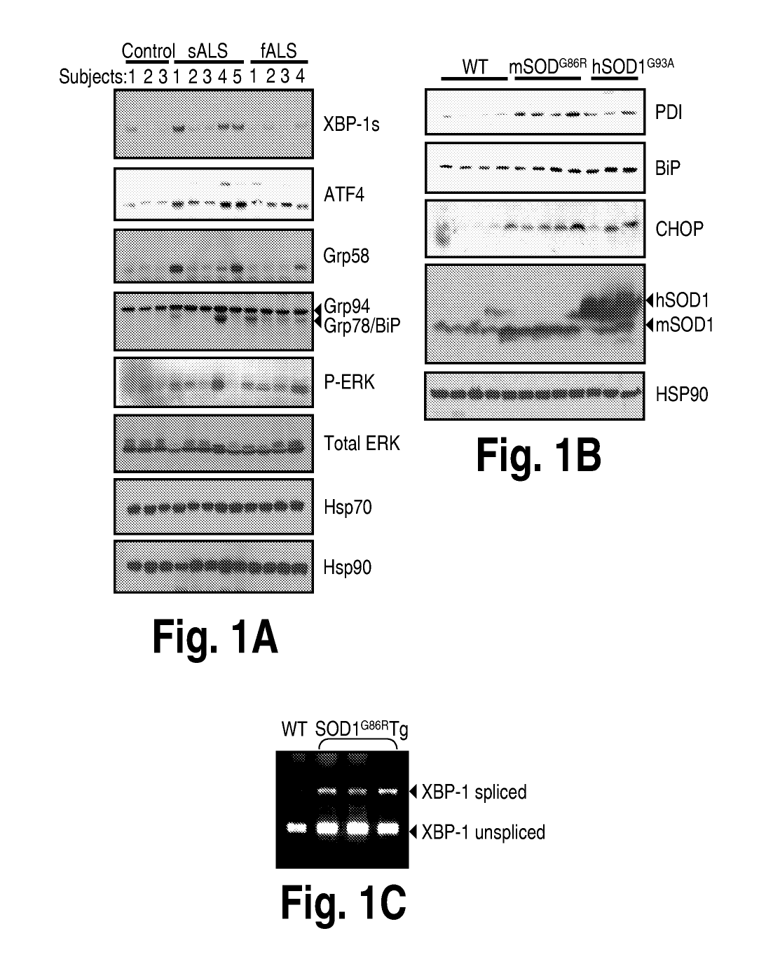

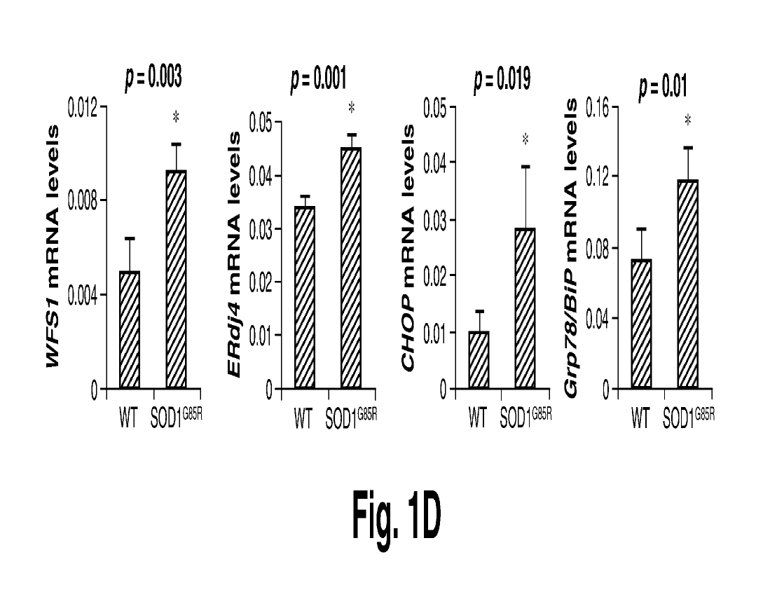

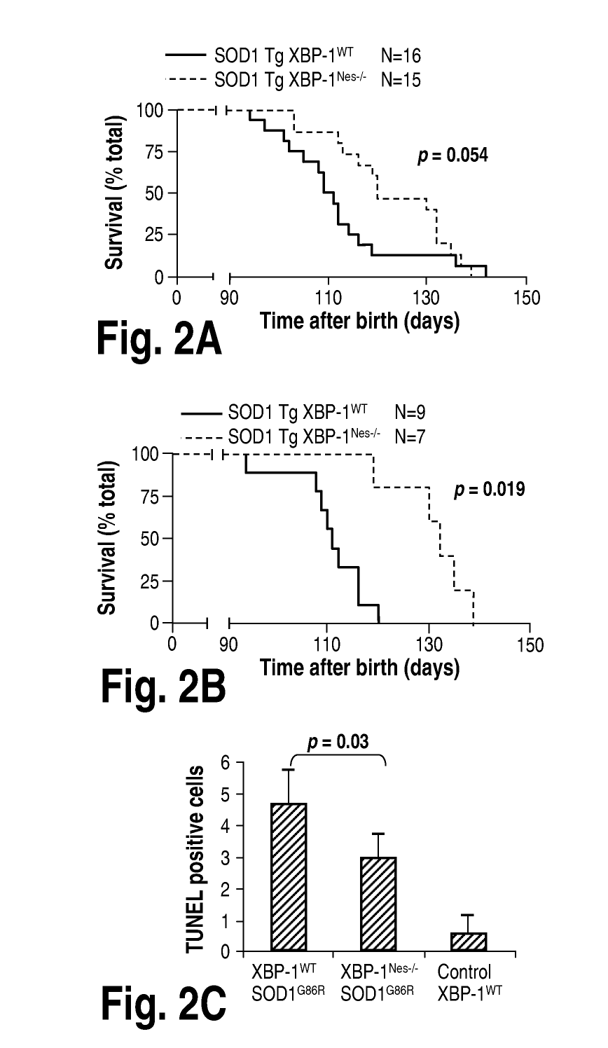

FIGS. 7(A)-(G) depict that XBP-1 deficiency increases autophagy in mutant SOD1 transgenic mice. (A) The levels of central autophagy regulators Beclin-1 and LC3 were investigated in total protein extracts derived from spinal cord post mortem samples from different patients affected with sALS and fALS. LC3 processing into the active LC3-II form is indicated. In addition, total levels of poly-ubiquitinated proteins were determined. There healthy controls were also studied. All subjects analyzed are the same as in FIG. 1A. (B) The expression levels of the Beclin-1, LC3 and Hsp90 were determined in spinal cord protein extracts from symptomatic mSOD1^{G86R} transgenic or control mice by Western blot. Two different animals are shown per group. (C) Autophagosomes were directly observed in the spinal cord of control, or mSOD1^{G86R} transgenic mice on an XBP-1^{Nes-/-} background by immuno-fluorescence using an anti-LC3 antibody. Neurons were co-stained with an anti-NeuN antibody. Motoneurons were identified by their morphology and staining with NeuN (white arrows). Total cells were stained with Hoechst (nucleus). Images are representative on the analysis of 5 different animals per group (FIG. 11). (D) In parallel, lysosomes were visualized in the samples presented in (B) after LAMP-2 staining. Co-localization with motoneurons was also evaluated after ChAT staining (white arrow). Total cells were stained with Hoechst (nucleus). Images are representative on the analysis of 5 different animals per group. (E) The pattern of total ubiquitinated proteins was analyzed by Western blot in SOD1^{G86R} and control mice breed in an XBP-1^{WT} and XBP-1^{Nes-/-} genetic background. Two control and four transgenic mice per group are shown. (F) EDEM was knockdown with shRNA in NSC34 and then mutant SOD1 aggregation was analyzed by Western blot analysis. Two different shRNA constructs against EDEM mRNA were employed. (G) In parallel, the content of autophagosomes was visualized in the same cells after the expression of LC3-EGFP.

FIGS. 8(A)-(D) show that knocking-down XBP-1/IRE-1 alpha decreases the levels of mutant SOD1 and poly(Q)₇₂ peptides. (A) NSC34 stably expressing shRNA constructs against IRE-1 alpha, XBP-1 or control mRNA were transiently transfected with expression vectors for human SOD1^{WT}-, SOD 1^{G93A}-, and SOD1^{G85R}-EFGP fusion proteins. After 72 h, SOD1 intracellular inclusions were visualized by fluorescent microscopy. Low magnification pictures are presented. (B) Transfection efficiency was analyzed in shRNA cell lines by transfecting an EGFP expression vector and FACS analysis. (C) In parallel, detergent insoluble SOD1 protein aggregates were determined in cell extracts prepared in RIPA and analyzed by Western blot using anti-GFP antibody. Of note, high molecular weight SOD1 aggregates are observed in the analysis. shRNA cells: Mock (M), XBP-1 (X) or IRE1α (I). (D) Cell viability after ER stress induction was determined in shControl, shXBP-1 and shIRE1α NSC34 cells after indicated concentrations of tunicamycin, thapsigargin or brefeldin A. After 24 h cell viability was determined using the MTS assay.

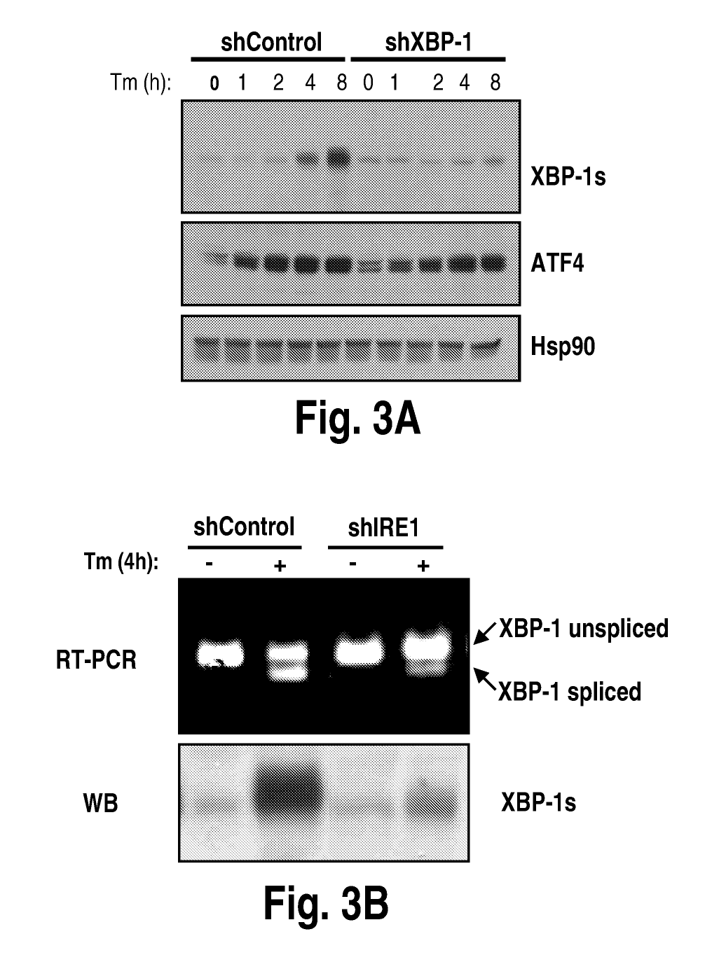

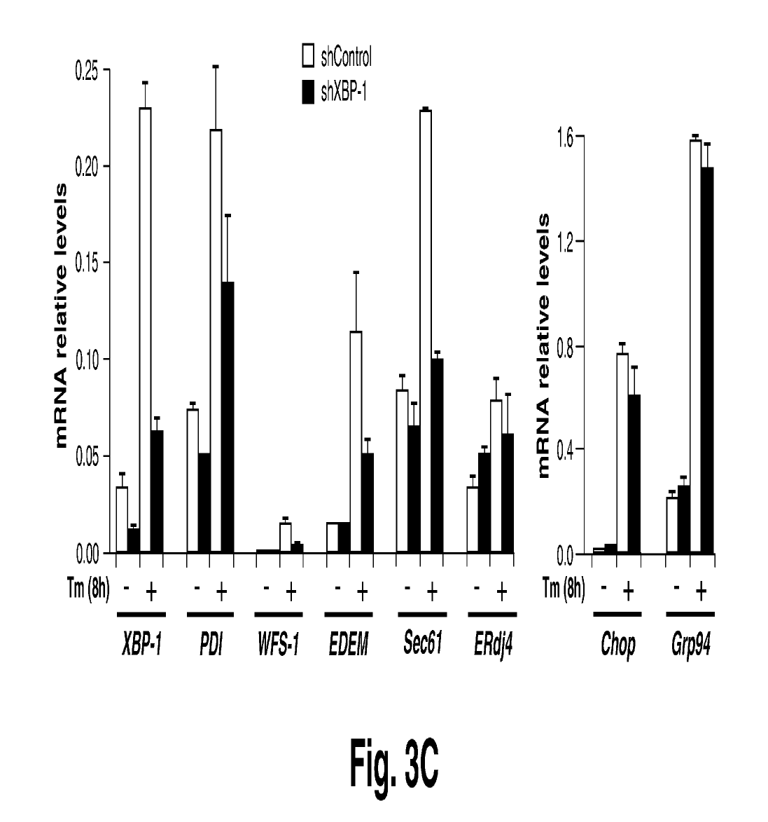

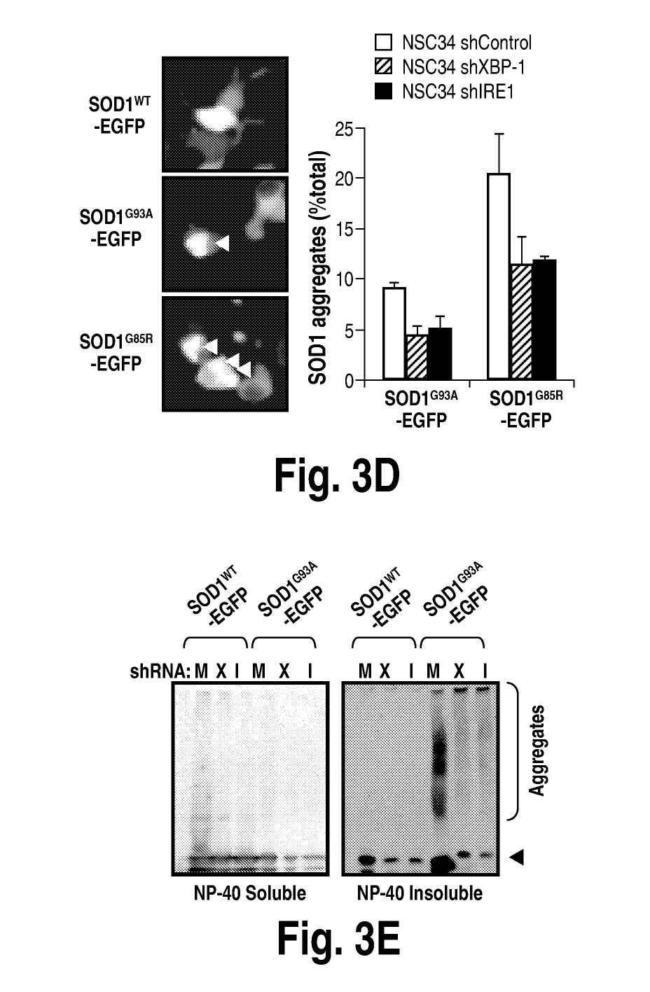

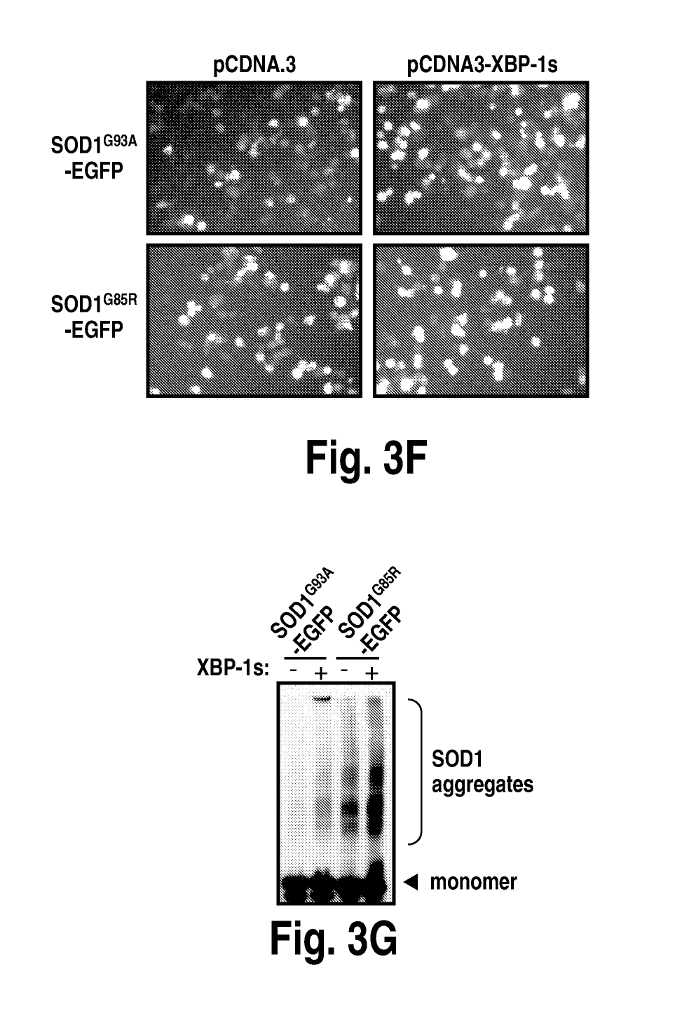

FIGS. 9(A)-(B) show that autophagy inhibition increases protein aggregation in XBP-1/IRE1α deficient cells. (A) The Western blot signal corresponding to high molecular weight SOD1 aggregates of FIG. 3A was quantified using a Versadoc machine and plotted as arbitrary units of band pixel intensity. (B) NSC34 cells expressing shRNA constructs against IRE1α and XBP-1 were transfected with an expression vector for poly(Q)₇₂-EGFP and after 48 h cells were treated for 16 h with 10 mM 3-MA or left untreated. Then, the generation of poly(Q)₇₂ intracellular inclusions were observed by fluorescent microscopy.

FIGS. 10(A)-(C) show colocalization between SOD1^{G8}5 and poly(Q)₇₂ intracellular inclusions with acidic compartments. (A) NSC34 shControl or shXBP-1 cells were transiently transfected with expression vectors for SOD1G85R-EFGP. After 72 h, cells were stained with lysotracker, and the co-localization with SOD1 intracellular inclusions was determined by confocal microscopy. Nucleus was stained with Hoechst. Representative pictures are presented to depict reproducibility of the observations. (B) NSC34 cells were transfected with an poly(Q)₇₂ expression vector and analyzed as described in (A). (C) Expression of Parkinson-related α-synuclein mutants does not increase the content of lysosomes. NSC34 cells transfected with α-synucleinWT, α-synuclein^{A53T} or α-synuclein^{E46K} as EGFP fusion proteins and after 48 h of transfection, the content of lysosomes was visualized after lysotracker staining and confocal microscopy analysis. Nucleus was stained with Hoechst. Six representative pictures are presented.

FIG. 11 shows that symptomatic SOD1 mice have increased autophagposome content which is further augmented in XBP-1 deficiency. Autophagosomes were directly observed in the spinal cord of control XBP-1^{WT} or XBP-1^{Nes-/-}, and mSOD1^{G86R} transgenic mice on an XBP-1^{Nes-/-} background by immuno-fluorescence using an anti-LC3 antibody. Neurons were co-stained with an anti-NeuN antibody. Motoneurons were identified by their morphology and staining with neuN. Total cells were stained with Hoechst (nucleus). Representative images from five different animals per group are presented. White arrows indicate neurons containing autophagosomes.

### DETAILED DESCRIPTION

The instant invention is based, at least in part, on the discovery that XBP-1 deficieny protects against neurodegenerative disease associated with protein aggregation. In particular, although it has previously been observed that XBP-1 deficiency results in failure to handle ER stress in organs such as pancreas (Lee, A. H., Chu, et al. (2005). EMBO J. 24, 4368-4380) and B lymphocytes (Iwakoshi, N. N., et al. (2003). Nat. Immunol. 4, 321-329; Reimold, A. M., et al. (2001). Nature 412, 300-307), leading to apoptosis, the inventors have surprisingly discovered that XBP-1 deficiency in motor neurons in animal models having a neurodegenerative disease associated with protein aggregation, increases the life span of the animals and reduces motor neuron apoptosis.

Furthermore, the present invention is based, at least in part, on the discovery that reduced expression of XBP-1, IRE-1 alpha, and EDEM (e.g., using shRNAs) in in vitro models of neurodegenerative disease associated with protein aggregation, e.g., ALS and Huntington's disease (HD), decreases the accumulation of intracellular inclusions of protein aggregates associated with the neurodegenerative disease, increases cell survival (decreases cell, e.g., motor neuron cell, apoptosis), and increases the levels of autophagy in the cells, e.g., motor neuron cells.

These findings provide for methods to identify agents that modulate, e.g., decrease, the expression and/or activity of XBP-1, IRE-1 alpha, and/or EDEM. In addition, the invention provides for the use of agents that modulate the expression and/or activity of XBP-1, IRE-1 alpha, and EDEM as targets in therapy. The present invention further provides methods for determining whether a subject is at risk of or has developed a neurodegenerative disorder associated with aberrant protein aggregation.

Certain terms are first defined so that the invention may be more readily understood.

### I. DEFINITIONS

As used herein “a neurodegenerative disease associated with protein aggregation” also referred to as “protein aggregation disorders”, “protein conformation disorders”, or “proteinopathies” include diseases or disorders characterised by the formation of detrimental intracellular protein aggregates (e.g., inclusions in the cytosol or nucleus) or extracellular protein aggregates (e.g., plaques). “Detrimental protein aggregation” is the undesirable and harmful accumulation, oligomerization, fibrillization or aggregation, of two or more, hetero- or homomeric, proteins or peptides. A detrimental protein aggregate may be deposited in bodies, inclusions or plaques, the characteristics of which are often indicative of disease and contain disease-specific proteins. For example, superoxide dismutase-1 aggregates are associated with ALS, poly-Q aggregates are associated with Huntington's disease, and α-synuclein-containing Lewy bodies are associated with Parkinson's disease. Non-limiting classes of Protein Aggregation Disorders or Proteopathies include Protein Conformational Disorders, Alpha-Synucleinopathies, Polyglutamine Diseases, Serpinopathies, Tauopathies or other related disorders. Non-limiting examples of Protein Aggregation Disorders include Amyotrophic Lateral Sclerosis (ALS), Huntington's Disease (HD), Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCAT), spinocerebellar ataxia type 17 (SCA17), chronic liver diseases, familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, or subacute sclerosing panencephalitis.

In one embodiment, a disease associated with protein aggregation is a neurogenerative disease associated with intracellular detrimental protein aggregates. In one embodiment, the neurodegenerative disease associated with protein aggregation is a disease associated with extracellular detrimental protein aggregates.

In one embodiment, the neurodegenerative disease associated with protein aggregation is Amyotrophic Lateral Sclerosis (ALS). In one embodiment, the ALS is familial ALS (fALS). In another embodiment, the ALS is sporadic ALS. “Amyotrophic lateral sclerosis” (“ALS”) is a prevalent, adult-onset neurodegenerative disease selective to motor neurons connecting the brain to the spinal cord and spinal cord to muscles. The neurons typically affected are located in the lower motor neurons of the brainstem and spinal cord and upper motor neurons in the cerebral cortex.

Within 2 to 5 years after clinical onset, the loss of motor neurons leads to progressive atrophy of skeletal muscles, which results in loss of muscular function resulting in paralysis, speech deficits, and death due to respiratory failure. The genetic defects that cause or predispose ALS onset are unknown, although missense mutations in the SOD-1 gene occur in approximately 10% of familial ALS cases, of which up to 20% have mutations in the gene encoding “superoxide dismutase” (“SOD1”), located on chromosome 21. SOD-1 normally functions in the regulation of oxidative stress by conversion of free radical superoxide anions to hydrogen peroxide and molecular oxygen. To date, over 90 mutations have been identified spanning all exons of the SOD-1 gene.

In another embodiment, the neurodegenerative disease associated with protein aggregation is Huntington's disease. “Huntington's disease” (“HD”) is an autosomal dominant, fully penetrant, neurodegenerative disease resulting from mutation in the Huntington gene. The mutation is an expansion of a trinucleotide repeat (CAG) in exon 1 of the HD gene, resulting in a “polyglutamine” (“polyQ”) expansion in the Huntingtin protein. The resulting gain of function is the basis for the pathological, clinical and cellular sequelae of Huntington's disease. Neuropathologically, the most striking changes occur in the caudate nucleus and putamen, where the medium spiny neurons are particularly vulnerable. Clinically, Huntington's disease is characterized by an involuntary choreiform movement disorder, psychiatric and behavioral chances and dementia. The age of onset is usually between the thirties and fifties, although juvenile and late onset cases of HD occur.

At the cellular level, Huntington's disease is characterized by protein aggregation in the cytoplasm and nucleus of neurons which comprise ubiquitinated terminal fragments of Huntingtin. (Bence et al., Science 292: pp. 1552-1555 (2001); Waelter et al., Molecular Biology of the Cell 12: pp. 1393-1407 (2001)).

The endoplasmic reticulum (ER) is a specialized cytosolic organelle in which various metabolic signals and pathways are integrated to regulate lipid, glucose, cholesterol, and protein metabolism. The ER is a principal site of protein synthesis, and together with the Golgi apparatus, it facilitates the transport and release of correctly folded proteins. Both surface and secreted proteins are synthesized in the ER where they need to fold and assemble prior to being transported.

Ribosomes attached to the ER membrane translate de novo peptides into the luminal space. Within the lumen of the ER, protein chaperones such as BiP (GRP78), calnexin, and calreticulin assist in the proper folding of de novo peptides and prevent the aggregation of unfolded or misfolded precursors. Once conformationally sound, the proteins are released to the Golgi for final modifications (such as oligosaccharide processing) and transported to their cellular destinations.

As used herein, the term “protein folding or transport” encompasses posttranslational processes including folding, glycosylation, subunit assembly and transfer to the Golgi compartment of nascent polypeptide chains entering the secretory pathway, as well as extracytosolic portions of proteins destined for the external or internal cell membranes, that take place in the ER lumen. Proteins in the ER are destined to be secreted or expressed on the surface of a cell. Accordingly, expression of a protein on the cell surface or secretion of a protein can be used as indicators of protein folding or transport.

Under normal cellular condition, e.g., when the cell is not under ER stress (described below), the ER operates a quality control system that identifies misfolded proteins, transports them into the cytoplasm and then targets them for degradation by the proteasome. The pathways that orchestrate the destruction of aberrant proteins are collectively termed “ER associated degradation” (“ERAD”), which includes, for example, “ER-associated glycoprotein degradation” (“GERAD”) and “ubiquitin-proteasome degradation” (discussed below).

More specifically, most of the proteins synthesized in the ER are cotranslationally modified with N (asparagine)-linked glycans by addition of triglucosylated, branched oligosaccharides at asparagines residues. Rapid glucose trimming by ER glucosidases generates monoglucosylated N-glycans (Glc0-3Man9GlcNAc2). Productive folding of the polypeptides to which the glycans are attached requires the services of an ER lectin chaperone called calnexin (CNX) that recognize the Glc1Man9GlcNAc2 oligosaccharide and assist the folding of newly synthesized glycoproteins. Beause the half-time of monoglucose in the nascent glycoproteins is about 2 minutes, the protein should be repeatedly reglycosylated by UDPglucose: glycoprotein glucosyltransferase (UUGT) resulting in association with CNX. Next, the mannose residue from the middle branch of the Man9GlcNAc2 oligosaccharide is removed by ER-mannosidase I, then correctly folded glycoproteins bearing Man8GlcNAc2 isomer B (Man8B form) are transported out of the ER to the Golgi apparatus.

If, however, the native structure of the glycoprotein cannot be achieved it is retained in the ER. When substrates are released from CNX by glucosidase II, they may be transferred to one of 2 pathways: EDEM for degradation or UUGT for reentry into CNX cycle. A glycoprotein with permanent structural defects is modified by the slow-acting enzyme ER mannosidase I. This enzyme attaches Man8 glycans to misfolded glycoproteins and in so doing generates a signal that results in ER-associated glycoprotein degradation (GERAD). If the glycoprotein has a glycan in the form Glc0-3Man8GlcNAc2, it is released from the calnexin cycle, recognised by a mannosidase-like protein EDEM and dislocated from the ER to the cytosol. Here the protein binds to the glycan-specific F box protein Fbx2 and is targeted to the SCF-Fbx2-type E3 ubiquitin ligase complex. This results in the protein being polyubiquitinated and target to the proteasome pathway. The protein is subsequently deglycosylated by a cytosolic peptide N-glycanase (PNGase) that is closely associated with the ubiquitin ligase complex and/or the 19S lid of the proteasome. Finally, the ubiquitinated, deglycosylated protein is degraded by the proteasome (ubiquitin-proteasome degradation).

Accordingly, as referred to herein, the term “proteasome pathway” or “ubiquitin-proteasome pathway” refers to a pathway by which a variety of cellular proteins are degraded. Many proteins are marked for degradation in this pathway by covalent attachment of ubiquitin. For example, the XBP-1 unspliced protein is an example of an ubiquitinated protein, and hence extremely unstable. XBP-1 spliced protein is not ubiquitinated, and has a much longer half life than unspliced XBP-1 protein.

As used herein “EDEM” or “endoplasmic reticulum degradation-enhancing alpha-mannosidase-like protein” is an enzymatically inactive mannosidase-like protein that accelerates the degradation of misfolded proteins in the ER and interacts with calnexin. EDEM extracts misfolded glycoproteins, but not glycoproteins undergoing productive folding, from the calnexin cycle. Overexpression of EDEM has been shown to result in faster release of folding-incompetent proteins from the calnexin cycle and earlier onset of degradation, whereas EDEM downregulation has been shown to prolong folding attempts and delay ER-associated degradation (ERAD). Furthermore, under ER stress conditions, EDEM is specifically upregulated by XBP-1 (Molinari, et al. Science 299: 1397-1400, 2003). gi:109472420 (rat EDEM).

There are two variants of human calnexin, the amino acid and nucleotide sequences of which may be found in, for example, GenBank accession Nos. gi:66933004 and gi:66933003. There are three variants of murine calnexin, the amino acid and nucleotide sequences of which may be found in, for example, gi:160333210, gi:160333211, and gi:160333215 3. The nucleotide and amino acid sequence of rat calnexin may be found in, for example, GenBank accession number: gi:164519098.

The nucleotide and amino acid sequence of human EDEM may be found in, for example, GenBank accession number: gi:7662001; the nucleotide and amino acid sequence of murine EDEM may be found in, for example, GenBank accession number: gi:119709811; the nucleotide and amino acid sequence of rat EDEM may be found in, for example, GenBank accession number gi:109472420.

In addition to the ERAD pathways for degradation of misfolded proteins described above (GERAD and the ubiquitin-proteasome pathway which predominantly degrade glycoproteins and short-lived nuclear and cytosolic proteins, respectively), the autophagy-lysosomal pathway is responsible for the bulk degradation of cytoplasmic proteins or organelles and is mediated by “macroautophagy”, also referred to as “autophagy”. “Autophagy” involves the formation of double membrane structures called “autophagosomes” or “autophagic vacuoles”, which encapsulates cytoplasm and organelles and then fuses with the primary lysosomes to form “autolysosomes” where their contents are degraded by acidic lysosomal hydrolases and then either disposed off or recycled back to the cell (Rubinsztein, D. C. (2006) Nature 443, 780-786). The formation of the double-membraned vesicle is a complex process involving 16 autophagy-related proteins (“Atg” proteins; see, e.g., Yorimitsu T, et al Cell Death Differ 2005; 12 Suppl 2:1542-52, the contents of which are incorporated by reference).

Under conditions of cellular stress leading to an impairment of ER function, proteins are unable to fold properly and accumulate in the ER lumen. It is to these unfolded or misfolded proteins that the ER has evolved a coping system known as the Unfolded Protein Response (UPR).

As used herein, the term “ER stress” includes conditions such as the presence of reducing agents, depletion of ER lumenal Ca2+, inhibition of glycosylation or interference with the secretory pathway (by preventing transfer to the Golgi system), which leads to an accumulation of misfolded protein intermediates and increase the demand on the chaperoning capacity, and induce ER-specific stress response pathways. ER stress pathways involved with protein processing include the UPR and the Endoplasmic Reticulum Overload Response (EOR) which is triggered by certain of the same conditions known to activate UPR (e.g. glucose deprivation, glycosylation inhibition), as well as by heavy overexpression of proteins within the ER. The distinguishing feature of EOR is its association with the activation of the transcription factor NF-κB. Modulation of both the UPR and the EOR can be accomplished using the methods and compositions of the invention. ER stress can be induced, for example, by inhibiting the ER Ca2+ATPase, e.g., with thapsigargin.

As used herein, the term “Unfolded Protein Response” (UPR) or the “Unfolded Protein Response pathway” refers to an adaptive response to the accumulation of unfolded proteins in the ER and includes the transcriptional activation of genes encoding chaperones and folding catalysts and protein degrading complexes as well as translational attenuation to limit further accumulation of unfolded proteins. It should be noted that the pathways for the degradation of misfolded proteins described above, ERAD and the autophagy-lysosomal pathway, are also operational under conditions of ER stress and respond to cellular signals described below by, for example upregulating the degradation of misfolded proteins.

Since the ER and the nucleus are located in separate compartments of the cell, the unfolded protein signal must be sensed in the lumen of the ER and transferred across the ER membrane and be received by the transcription machinery in the nucleus. The unfolded protein response (UPR) performs this function for the cell. Activation of the UPR can be caused by treatment of cells with reducing agents like DTT, by inhibitors of core glycosylation like tunicamycin or by Ca-ionophores that deplete the ER calcium stores. First discovered in yeast, the UPR has now been described in C. elegans as well as in mammalian cells. In mammals, the UPR functions via signaling through three arms or branches, denoted for the three stress-sensing proteins found in the ER membrane: PKR-like eukaryotic initiation factor 2α kinase (PERK), inositol-requiring enzyme-1 (IRE-1 alpha), and activating transcription factor-6 (ATF-6). These three transmembrane proteins are normally bound by the ER chaperone BiP in their intraluminal domains. When client proteins (also bound by BiP) begin to exceed ER capacity, less BiP is available for binding to the UPR sensors. Without BiP binding, PERK and IRE-1 alpha auto-oligomerize and undergo autophosphorylation, leading to the activation of downstream signaling. ATF-6 is released to the Golgi, it undergoes two subsequent cleavages to produce an active transcription factor.

One result of PERK activation is selective attenuation of protein translation through inhibitory phosphorylation of eukaryotic translational initiation factor 2{ alpha} (eIF2{alpha}) at serine 51. This phosphorylation also results in an increased alternative translation of ATF-4, which induces the expression of many genes, including those involved in apoptosis [C/EBP homologous protein (CHOP)]. ER redox control [endoplasmic reticulum oxidoreductin (ERO1)], and the negative feedback release of eIF2{alpha} inhibition [growth arrest and DNA damage-inducible protein (GADD34)]. PERK signaling also results in an antioxidant response mediated by the activated transcription factor Nrf2 (nuclear erythroid 2 p45-related factor 2).

In addition to selective inhibition of de novo protein synthesis, the UPR also induces the transcription of chaperones to assist with the unfolded protein load. Activated ATF-6 translocates to the nucleus and upregulates the gene expression of chaperones such as BiP, calreticulin, and GRP94. The process of ER-associated degradation is also upregulated at this time to facilitate the clearance and degradation of excess client proteins from the ER lumen. ATF-6 induces the expression of ER degradation-enhancing {alpha}-mannosidase-like protein (EDEM), which is involved in this process. ATF-6 also upregulates X-box protein 1 (XBP-1) mRNA, which is further processed and specially regulated by the IRE-1 alpha response arm.

IRE-1 alpha activation by the UPR also contributes to the increase in protein chaperone content as well as to ER biogenesis and enhanced secretory capacity via the action of XBP-1. IRE-1 alpha, acting as an endoribonuclease, cleaves a 26 bp segment out of the mRNA of XBP-1, creating a spliced mRNA that translates an active form of the transcription factor (XBP-1s). XBP-1s, in turn, induces the expression of protein chaperones, as well as proteins involved in ER biogenesis and secretion (e.g., EDEM, endoplasmic reticulum-resident DNAj homolog 4 (ERdj4), protein disulfide isomerase (PDI), and ER proteins), and acts as one of the major pathways regulating ER function and folding capacity.

As described above, eukaryotic cells respond to the presence of unfolded proteins by upregulating the transcription of genes encoding ER resident protein chaperones such as the glucose-regulated BiP/Grp74, GrP94 and CHOP genes, folding catalysts and protein degrading complexes that assist in protein folding. As used herein, the term “modulation of the UPR” includes both upregulation and downregulation of the UPR. As used herein the term “UPRE” refers to UPR elements upstream of certain genes which are involved in the activation these genes in response, e.g., to signals sent upon the accumulation of unfolded proteins in the lumen of the endoplasmic reticulum.

These cellular processes described above are for the recovery and survival of the cell. However, if the UPR cannot adapt to the presence of unfolded proteins in the ER, an apoptotic response is initiated leading to the activation of JNK protein kinase and caspases 7, 12, and 3.

As used herein the term “apoptosis” includes programmed cell death which can be characterized using techniques which are known in the art. Apoptotic cell death can be characterized, e.g., by cell shrinkage, membrane blebbing and chromatin condensation culminating in cell fragmentation. Cells undergoing apoptosis also display a characteristic pattern of internucleosomal DNA cleavage. As used herein, the term “modulates apoptosis” includes either up regulation or down regulation of apoptosis in a cell.

As used herein, the term “XBP-1” refers to the X-box binding protein. XBP-1 is a basic region leucine zipper (b-zip) transcription factor isolated independently by its ability to bind to a cyclic AMP response element (CRE)-like sequence in the mouse class II MHC Ac gene or the CRE-like site in the HTLV-1 21 base pair enhancer, and subsequently shown to regulate transcription of both the DRα and HTLV-1 ltr gene.

Like other members of the b-zip family, XBP-1 has a basic region that mediates DNA-binding and an adjacent leucine zipper structure that mediates protein dimerization. Deletional and mutational analysis has identified transactivation domains in the C-terminus of XBP-1 in regions rich in acidic residues, glutamine, serine/threonine and proline/glutamine. XBP-1 is present at high levels in plasma cells in joint synovium in patients with rheumatoid arthritis. In human multiple myeloma cells, XBP-1 is selectively induced by IL-6 treatment and implicated in the proliferation of malignant plasma cells. XBP-1 has also been shown to be a key factor in the transcriptional regulation of molecular chaperones and to enhance the compensatory UPR (Calfon et al., Nature 415, 92 (2002); Shen et al., Cell 107:893 (2001); Yoshida et al., Cell 107:881 (2001); Lee et al., Mol. Cell Biol. 23:7448 (2003); each of which is incorporated herein by reference).

The amino acid sequence of XBP-1 is described in, for example, Liou, H-C. et. al. (1990) Science 247:1581-1584 and Yoshimura, T. et al. (1990) EMBO J. 9:2537-2542. The amino acid sequence of mammalian homologs of XBP-1 are described in, for example, in Kishimoto T. et al., (1996) Biochem. Biophys. Res. Commun. 223:746-751 (rat homologue). Exemplary nucleic acid molecules and polypeptides intended to be encompassed by the term “XBP-1” include those having disclosed in GenBank accession numbers gi:118640872 (human XBP-1 spliced (XBP-1s)); gi:172072591 (human XBP-1 unspliced (XBP-1u)); gi:51948391 (rat XBP-1); gi:13775155 (mouse XBP-1). XBP-1 is also referred to in the art as TREB5 or HTF (Yoshimura et al. 1990. EMBO Journal. 9:2537; Matsuzaki et al. 1995. J. Biochem. 117:303).

There are two forms of XBP-1 protein, unspliced and spliced, which differ markedly in their sequence and activity. Unless the form is referred to explicitly herein, the term “XBP-1” as used herein includes both the spliced and unspliced forms.

As used herein, the term “spliced XBP-1” or “XBP-1s” refers to the spliced, processed form of the mammalian XBP-1 mRNA or the corresponding protein. Human and murine XBP-1 mRNA contain an open reading frame (ORF1) encoding bZIP proteins of 261 and 267 amino acids, respectively. Both mRNAs also contain another ORF, ORF2, partially overlapping but not in frame with ORF1. ORF2 encodes 222 amino acids in both human and murine cells. Human and murine ORF1 and ORF2 in the XBP-1 mRNA share 75% and 89% identity respectively. In response to ER stress, XBP-1 mRNA is processed by the ER transmembrane endoribonuclease and kinase IRE-1 alpha which excises an intron from XBP-1 mRNA. In murine and human cells, a 26 nucleotide intron is excised. The boundaries of the excised introns are encompassed in an RNA structure that includes two loops of seven residues held in place by short stems. The RNA sequences 5′ to 3′ to the boundaries of the excised introns form extensive base-pair interactions. Splicing out of 26 nucleotides in murine and human cells results in a frame shift at amino acid 165 (the numbering of XBP-1 amino acids herein is based on GenBank accession number gi:13775155 and one of ordinary skill in the art can determine corresponding amino acid numbers for XBP-1 from other organisms, e.g., by performing a simple alignment). This causes removal of the C-terminal 97 amino acids from the first open reading frame (ORF1) and addition of the 212 amino from ORF2 to the N-terminal 164 amino acids of ORF1 containing the b-ZIP domain. In mammalian cells, this splicing event results in the conversion of a 267 amino acid unspliced XBP-1 protein to a 371 amino acid spliced XBP-1 protein. The spliced XBP-1 then translocates into the nucleus where it binds to its target sequences to induce their transcription.

As used herein, the term “unspliced XBP-1” refers to the unprocessed XBP-1 mRNA or the corresponding protein. As set forth above, unspliced murineXBP-1 is 267 amino acids in length and spliced murine XBP-1 is 371 amino acids in length. The sequence of unspliced XBP-1 is known in the art and can be found, e.g., Liou, H-C. et. al. (1990) Science 247:1581-1584 and Yoshimura, T. et al. (1990) EMBO J. 9:2537-2542, or at GenBank accession numbers: AF443192 [gi: 18139942] (amino acid spliced murine XBP-1); AF027963 [gi: 13752783] (amino acid murine unspliced XBP-1); NM_{—}013842 [gi:13775155] (nucleic acid spliced murine XBP-1); or M31627 [gi:184485] (nucleic acid unspliced murine XBP-1).

As used herein, the term “ratio of spliced to unspliced XBP-1” refers to the amount of spliced XBP-1 present in a cell or a cell-free system, relative to the amount or of unspliced XBP-1 present in the cell or cell-free system. “The ratio of unspliced to spliced XBP-1” refers to the amount of unspliced XBP-1 compared to the amount of unspliced XBP-1. “Increasing the ratio of spliced XBP-1 to unspliced XBP-1” encompasses increasing the amount of spliced XBP-1 or decreasing the amount of unspliced XBP-1 by, for example, promoting the degradation of unspliced XBP-1. Increasing the ratio of unspliced XBP-1 to spliced XBP-1 can be accomplished, e.g., by decreasing the amount of spliced XBP-1 or by increasing the amount of unspliced XBP-1. Levels of spliced and unspliced XBP-1 an be determined as described herein, e.g., by comparing amounts of each of the proteins which can be distinguished on the basis of their molecular weights or on the basis of their ability to be recognized by an antibody. In another embodiment described in more detail below, PCR can be performed employing primers with span the splice junction to identify unspliced XBP-1 and spliced XBP-1 and the ratio of these levels can be readily calculated.

As used herein, the term “IRE-1 or IRE-1 alpha” refers to refers to an ER transmembrane endoribonuclease and kinase called inositol requiring enzyme, which oligomerizes and is activated by autophosphorylation upon sensing the presence of unfolded proteins, see, e.g., Ron and Walter 2007. Nat. Rev. Mol. Cell Biol. 8:519. In Saccharomyces cerevisiae, the UPR is controlled by IREp. In the mammalian genome, there are two homologs of IRE-1 alpha, IRE1α and IRE1β. IRE1α is expressed in all cells and tissue whereas IRE1β is primarily expressed in intestinal tissue. The endoribonucleases of either IRE1α and IRE1β are sufficient to activate the UPR. Accordingly, as used herein, the term “IRE-1” includes, e.g., IRE1α, IRE1β and IREp. In a preferred embodiment, IRE-1 refers to IRE1α.

Over-expression of the IRE-1 alpha gene leads to constitutive activation of the UPR. Phosphorylation of the IRE-1 alpha protein occurs at specific serine or threonine residues in the protein. IRE-1 alpha senses the overabundance of unfolded proteins in the lumen of the ER. The oligomerization of this kinase leads to the activation of a C-terminal endoribonuclease by trans-autophosphorylation of its cytoplasmic domains. IRE-1 alpha uses its endoribonuclease activity to excise an intron from XBP-1 mRNA. Cleavage and removal of a small intron is followed by re-ligation of the 5′ and 3′ fragments to produce a processed mRNA that is translated more efficiently and encodes a more stable protein (Calfon et al. (2002) Nature 415(3): 92-95). The nucleotide specificity of the cleavage reaction for splicing XBP-1 is well documented and closely resembles that for IRE-p mediated cleavage of HAC1 mRNA (Yoshida et al. (2001) Cell 107:881-891). In particular, IRE-1 alpha mediated cleavage of murine XBP-1 cDNA occurs at nucleotides 506 and 532 and results in the excision of a 26 base pair fragment (e.g., CAGCACTCAGACTACGTGCACCTCTG (SEQ ID NO:1) for mouse XBP-1. IRE-1 alpha mediated cleavage of XBP-1 derived from other species, including humans, occurs at nucleotides corresponding to nucleotides 506 and 532 of murine XBP-1 cDNA, for example, between nucleotides 502 and 503 and 528 and 529 of human XBP-1. The nucleotide and amino acid sequences of IRE-1 alpha are kown in the art and can be found in, for example, GenBank accession No. gi:153946420 (human IRE-1); gi:15284149 (mouse IRE-1); gi:109489193 (rat IRE-1).

XBP-1 activation is known to control expression of several other genes, for example, ERdj4, p58ipk, EDEM, PDI-P5, RAMP4, HEDJ, BiP, ATF6α, XBP-1, Armet and DNAJB9, which encodes the 222 amino acid protein, mDj7 (see, e.g., :ee et al. 2003b. Mol Cell Biol 23:7448), Grp58, Sec61, Wfs-1, Chop, Herp. These genes are important in a variety of cellular functions. For example, Hsp70 family proteins including BiP/Grp78 which is a representative ER localizing HSP70 member, function in protein folding in mammalian cells. A family of mammalian DnaJ/Hsp40-like proteins has recently been identified that are presumed to carry out the accessory folding functions. Two of them, Erdj4 and p58ipk, were shown to be induced by ER stress, localize to the ER, and modulate HSP70 activity (Chevalier et al. 2000 J Biol Chem 275: 19620-19627; Ohtsuka and Hata 2000 Cell Stress Chaperones 5: 98-112; Yan et al. 2002 Proc Natl Acad Sci USA 99: 15920-15925). ERdj4 has recently been shown to stimulate the ATPase activity of BiP, and to suppress ER stress-induced cell death (Kurisu et al. 2003 Genes Cells 8: 189-202; Shen et al. 2003 J Biol Chem 277: 15947-15956). ERdj4, p58IPK, EDEM, RAMP-4, PDI-P5 and HEDJ, all appear to act in the ER. ERdj4 (Shen et al. 2003), p58IPK (Melville et al. 1999 J Biol Chem 274: 3797-3803) and HEDJ (Yu et al. 2000 Mol Cell 6: 1355-1364) are localized to the ER and display Hsp40-like ATPase augmenting activity for the HspTO family chaperone proteins. EDEM was shown to be critically involved in the ERAD pathway by facilitating the degradation of ERAD substrates (Hosokawa et al. 2001 EMBO Rep 2:415-422; Molinari et al. 2003 Science 299 1397-1400; Oda et al. 2003 Science 299:1394-1397; Yoshida et al. 2003 Dev. Cell. 4:265-271). RAMP4 is a recently identified protein implicated in glycosylation and stabilization of membrane proteins in response to stress (Schroder et al. 1999 EMBO J 18:4804-4815; Wang and Dobberstein 1999 Febs Lett 457:316-322; Yamaguchi et al. 1999 J. Cell Biol 147:1195-1204). PDI-P5 has homology to protein disulfide isomerase, which is thought to be involved in disulfide bond formation (Kikuchi et al. 2002 J. Biochem (Tokyo) 132:451-455). Armet is also upregulated by the UPR and inhibits cell proliferation and ER stress-induced cell death (see, e.g., Apostoloua, et al. (2008) Exp Cell Res in press). Herp (homocysteine-induced endoplasmic reticulum protein) is an ER-resident membrane protein, which has a ubiquitin-like domain at its N-terminus. Expression of Herp protein is up-regulated in response to ER stress, including homocysteine. Herp stabilizes neuronal Ca2+ homeostasis and is involved in improving the balance of the folding capacity and protein loading in the ER (see, e.g., Lenz, et al. (2006) Exp Cell Res 312:4049). Expression of WFS1 protein, an ER resident membrane protein, is up-regulated by ER stress-inducing agents and is a component of the IRE1 and PERK signaling pathways. In addition, mutations in the gene encoding this protein are responsible for Wolfram syndrome (see, e.g., Fonseca et al (2005) J. Biol. Chem., 280, 39609). The SEC61 complex forms the core of the mammalian endoplasmic reticulum (ER) translocon, a transmembrane channel for the translocation (cotranslational or posttranslational transport) of proteins across the ER membrane protein and is composed of alpha, bet and gamma subunits. The SEC61 complex can also function in retrograde transport of multidomain integral membrane proteins from the ER to the cytosol for proteasomal degradation as part of ERAD and is upregulated during ER stress. CHOP is a C/EBP family transcription factor involved in endoplasmic reticulum (ER) stress-mediated apoptosis. Grp58 is an ER stress inducible chaperone protein. Collectively, these results show that the IRE1/XBP-1/EDEM pathway is required for efficient protein folding, maturation and degradation in the ER.

As used herein, the term “chaperone gene” is includes genes that are induced as a result of the activation of the UPR or the EOR. The chaperone genes include, for example, members of the family of Glucose Regulated Proteins (GRPs) such as GRP78 (BiP) and GRP94 (endoplasmin), as well as other chaperones such as calreticulin, protein disulfide isomerase, and ERp72. The upregulation of chaperone genes helps accommodate the increased demand for the folding capacity within the ER.

As used herein, the term “gene whose transcription is regulated by XBP-1”, includes genes having a regulatory region regulated by XBP-1. Such genes can be positively or negatively regulated by XBP-1. The term also includes genes which are indirectly regulated by XBP-1, e.g., are regulated by molecule in a signaling pathway in which XBP-1 is involved. Exemplary genes directly regulated by XBP-1 include, for example, genes such as ERdj4, p58ipk, EDEM, PDI-P5, RAMP4, BiP, ATF6α, XBP-1, Armet, DNAJB9, Grp58, Sec61, Wfs-1, Herp, Chop, the MHC class II genes (various MHC class II gene sequences are known in the art) and the IL-6 gene.

As discussed above, another UPR signaling pathway is activated by the PERK protein kinase. PERK phosphorylates eIF2α, which induces a transient suppression of protein translation accompanied by induction of transcription factor(s) such as ATF4 (Harding et al. 2000 Mol Cell 6: 1099-1108). eIF2α is also phosphorylated under various cellular stress conditions by specific kinases, double strand RNA activated protein kinase PKR, the amino acid control kinase GCN2 and the heme regulated inhibitor HRI (Samuel 1993 J. Biol. Chem 268:7603-76-6; Kaufman 1999 Genes Dev. 13: 1211-1233). Since genes that are induced by the PERK pathway are also induced by other stress signals, such as amino acid deprivation, it is likely that PERK dependent UPR target genes carry out common cellular defense mechanisms, such as cellular homeostasis, apoptosis and cell cycle (Harding et al. 2003 Mol. Cell 11619-633). Collectively, ER stress activates IRE/XBP-1 and PERK/eIF2α pathways to ensure proper maturation and degradation of secretory proteins and to effect common cellular defense mechanisms, respectively.

The reliance of p58IPK gene expression on XBP-1 connects two of the UPR signaling pathways, IRE1/XBP-1 and PERK. P581PK was originally identified as a 58 kD inhibitor of PKR in influenza virus-infected kidney cells (Lee et al. 1990 Proc Natl Acad Sci USA 87: 6208-6212) and described to downregulate the activity of PKR by binding to its kinase domain (Katze 1995 Trends Microbiol 3: 75-78). It also has a J domain in the C-terminus which has been shown to participate in interactions with Hsp70 family proteins Melville et al. 1999 J Biol Chem 274: 3797-380). Recently Katze and colleagues have demonstrated that p58IPK interacts with ERK which is structurally similar to PKR, inhibits its eIF2α kinase activity and that it is induced during the UPR by virtue of an ER stress-response element in its promoter region (Yan et al. 2002 Proc Natl Acad Sci USA 99: 15920-15925).

As used herein, the various forms of the term “modulate” include stimulation (e.g., increasing or upregulating a particular response or activity) and inhibition (e.g., decreasing or downregulating a particular response or activity).

As used herein, the terms “a modulator of XBP-1”, “a modulator of IRE-1 alpha”, and “a modulator of EDEM” include modulators of XBP-1 and/or IRE-1 alpha and/or expression and/or activity. The term includes agents, for example a compound or compounds which modulates transcription of, for example, an XBP-1, IRE-1 alpha, and/or EDEM gene, processing of an XBP-1 mRNA (e.g., splicing), translation of XBP-1 mRNA, post-translational modification of an XBP-1 and/or IRE-1 alpha protein (e.g., glycosylation, ubiquitination), or activity of an XBP-1, IRE-1 alpha, and/or EDEM protein. In one embodiment, a modulator modulates one or more of the above. In preferred embodiments, the activity of XBP-1, IRE-1 alpha, and/or EDEM is modulated. A “modulator of XBP-1 activity”, “a modulator of IRE-1 alpha activity” and “a modulator of EDEM activity” includes compounds that directly or indirectly modulate XBP-1, IRE-1 alpha, and/or EDEM activity. For example, an indirect modulator of XBP-1 activity can modulate a non-XBP-1 molecule which is in a signal transduction pathway that includes XBP-1. Examples of modulators that directly modulate XBP-1 expression, processing, post-translational modification, and/or activity include nucleic acid molecules encoding a biologically active portion of XBP-1, biologically active portions of XBP-1, nucleic acid molecules (e.g., antisense or siRNA nucleic acid molecules) that bind to XBP-1 mRNA or genomic DNA or otherwise reduce expression of XBP-1, intracellular antibodies that bind to XBP-1 intracellularly and modulate (i.e., inhibit) XBP-1 activity, XBP-1 peptides that inhibit the interaction of XBP-1 with a target molecule (e.g., IRE-1 alpha), as well as chemical compounds that act to specifically modulate the activity of XBP-1. Examples of modulators that directly modulate IRE-1 alpha expression, processing, post-translational modification, and/or activity include nucleic acid molecules encoding a biologically active portion of IRE-1, biologically active portions of IRE-1, antisense or siRNA nucleic acid molecules that bind to IRE-1 mRNA or genomic DNA, IRE-1 peptides that inhibit the interaction of IRE-1 with a target molecule (e.g., XBP-1), as well as chemical compounds that act to specifically modulate the activity of IRE-1. Examples of modulators that directly modulate EDEM expression, processing, post-translational modification, and/or activity include nucleic acid molecules encoding a biologically active portion of EDEM, biologically active portions of EDEM, antisense or siRNA nucleic acid molecules that bind to EDEM mRNA or genomic DNA, EDEM peptides that inhibit the interaction of EDEM with a target molecule, as well as chemical compounds that act to specifically modulate the activity of EDEM.

As used interchangeably herein, the terms “XBP-1 activity,” “biological activity of XBP-1” or “functional activity XBP-1,” include activities exerted by XBP-1 protein on an XBP-1 responsive cell or tissue, e.g., a motor neuron cell, or on an XBP-1 nucleic acid molecule or protein target molecule, as determined in vivo, or in vitro, according to standard techniques. XBP-1 activity can be a direct activity, such as an association with an XBP-1-target molecule e.g., binding of spliced XBP-1 to a regulatory region of a gene responsive to XBP-1 (for example, a gene such as ERdj4, p58^{ipk}, EDEM, PDI-P5, RAMP4, HEDJ, BiP, ATF6α, XBP-1, Armet and/or DNAJB9), Grp58, Sec61, Wfs-1, Herp, Chop, or the inhibition of spliced XBP-1 by unspliced XBP-1. Alternatively, an XBP-1 activity is an indirect activity, such as a downstream biological event mediated by interaction of the XBP-1 protein with an XBP-1 target molecule, e.g., EDEM. The biological activities of XBP-1 are described herein and include: e.g., modulation of a neurodegenerative diease associated with protein aggregation, modulation of autophagy, modulation of motor neuron survival, modulation of the generation of protein aggregation associated with a neurodegenerative disease, modulation of apoptosis, modulation of the UPR, modulation of the proteasome pathway, modulation of ERAD, and modulation of protein folding and transport.

As used interchangeably herein, the terms “IRE-1 alpha activity,” “biological activity of IRE-1 alpha” or “functional activity IRE-1 alpha,” include activities exerted by IRE-1 protein on an IRE-1 responsive cell or tissue, e.g., a motor neuron cell, or on an IRE-1 alpha nucleic acid molecule or protein target molecule, as determined in vivo, or in vitro, according to standard techniques. IRE-1 alpha activity can be a direct activity, such as an association with an IRE-1 alpha-target molecule e.g., IRE-1 alpha phosphorylation of a substrate (e.g., IRE-1 alpha autokinase activity) or endoribonuclease activity on a substrate e.g., XBP-1 mRNA. Alternatively, an IRE-1 alpha activity is an indirect activity, such as a downstream biological event mediated by interaction of the IRE-1 alpha protein with an IRE-1 alpha target molecule, e.g., XBP-1. As IRE-1 alpha is in a signal transduction pathway involving XBP-1, modulation of IRE-1 alpha modulates a molecule in a signal transduction pathway involving XBP-1. Modulators which modulate an XBP-1 biological activity indirectly modulate expression and/or activity of a molecule in a signal transduction pathway involving XBP-1, e.g., IRE-1 alpha, PERK, eIF2α, or ATF6α. The biological activities of IRE-1 alpha are described herein and include: e.g e.g., modulation of a neurodegenerative diease associated with protein aggregation, modulation of autophagy, modulation of motor neuron survival, modulation of the generation of protein aggregation associated with a neurodegenerative disease, modulation of apoptosis, modulation of the UPR, modulation of the proteasome pathway, modulation of ERAD, and modulation of protein folding and transport.

As used interchangeably herein, the terms “EDEM activity,” “biological activity of EDEM” or “functional activity EDEM” include activities exerted by EDEM protein on an EDEM responsive cell or tissue, e.g., a motor neuron cell, or on an EDEM nucleic acid molecule or protein target molecule, as determined in vivo, or in vitro, according to standard techniques. EDEM activity can be a direct activity, such as an association with an EDEM-target molecule e.g., calnexin. Alternatively, an EDEM activity is an indirect activity, such as a downstream biological event mediated by interaction of the EDEM protein with an EDEM target molecule, e.g., ubiquitinated cellular protein levels. As EDEM is in a signal transduction pathway involving XBP-1, modulation of EDEM modulates a molecule in a signal transduction pathway involving XBP-1. The biological activities of EDEM are described herein and include: e.g modulation of a neurodegenerative diease associated with protein aggregation, modulation of autophagy, modulation of motor neuron survival, modulation of the generation of protein aggregation associated with a neurodegenerative disease, modulation of apoptosis, modulation of the UPR, modulation of the proteasome pathway, modulation of ERAD, and modulation of protein folding and transport.

“Activity of unspliced XBP-1” includes the ability to modulate the activity of spliced XBP-1. In one embodiment, unspliced XBP-1 competes for binding to target DNA sequences with spliced XBP-1. In another embodiment, unspliced XBP-1 disrupts the formation of homodimers or heterodimers (e.g., with cfos or ATF6α) by XBP-1.

As used herein, a “substrate” or “target molecule” or “binding partner” is a molecule with which a protein binds or interacts in nature, such that that protein's function (e.g., modulation of e.g., modulation of a neurodegenerative diease associated with protein aggregates, autophagy, motor neuron survival, the generation of protein aggregation associated with a neurodegenerative disease, apoptosis, the UPR, the proteasome pathway, protein folding and transport) is achieved. For example, a target molecule can be a protein or a nucleic acid molecule. Exemplary target molecules of the invention include proteins in the same signaling pathway as the XBP-1, IRE-1 alpha, and/or EDEM protein, e.g., proteins which can function upstream (including both stimulators and inhibitors of activity) or downstream of the XBP-1, IRE-1 alpha, and/or EDEM protein in a pathway involving regulation of, for example, e.g., modulation of a neurodegenerative diease associated with protein aggregation, modulation of autophagy, modulation of motor neuron survival, modulation of the generation of protein aggregation associated with a neurodegenerative disease, modulation of apoptosis, modulation of the UPR, modulation of the proteasome pathway, modulation of ERAD, and modulation of protein folding and transport. Exemplary XBP-1 target molecules include IRE-1 alpha, ATF6α, XBP-1 itself (as the molecule forms homodimers), cfos (which can form heterodimers with XBP-1) as well as the regulatory regions of genes regulated by XBP-1. Exemplary IRE-1 alpha target molecules include XBP-1 and IRE-1 alpha itself (as the molecule can form homodimers).

The subject methods can employ various target molecules. For example, in one embodiment, the subject methods employ XBP-1, IRE-1 alpha, and/or EDEM. In another embodiment, the subject methods employ at least one other molecule, e.g., a molecule either upstream or downstream of XBP-1. For example, in one embodiment, the subject methods employ IRE-1 alpha. In another embodiment, the subject methods employ EDEM. In another embodiment, the subject methods employ EDEM and calnexin.

As used herein, the term “contacting” (i.e., contacting a cell e.g. a cell, with a compound) includes incubating the compound and the cell together in vitro (e.g., adding the compound to cells in culture) as well as administering the compound to a subject such that the compound and cells of the subject are contacted in vivo. The term “contacting” does not include exposure of cells to an XBP-1, IRE-1 alpha, and/or EDEM modulator that may occur naturally in a subject (i.e., exposure that may occur as a result of a natural physiological process).

As used herein, the term “test compound” refers to a compound that has not previously been identified as, or recognized to be, a modulator of the activity being tested. The term “library of test compounds” refers to a panel comprising a multiplicity of test compounds.

As used herein, the term “indicator composition” refers to a composition that includes a protein of interest (e.g., XBP-1), for example, a cell that naturally expresses the protein, a cell that has been engineered to express the protein by introducing an expression vector encoding the protein into the cell, or a cell free composition that contains the protein (e.g., purified naturally-occurring protein or recombinantly-engineered protein).

As used herein, the term “cell” includes prokaryotic and eukaryotic cells. In one embodiment, a cell of the invention is a bacterial cell. In another embodiment, a cell of the invention is a fungal cell, such as a yeast cell. In another embodiment, a cell of the invention is a vertebrate cell, e.g., an avian or mammalian cell. In a preferred embodiment, a cell of the invention is a murine or human cell.

As used herein, the term “engineered” (as in an engineered cell) refers to a cell into which a nucleic acid molecule e.g., encoding an XBP-1 protein (e.g., a spliced and/or unspliced form of XBP-1) has been introduced.

As used herein, the term “cell free composition” refers to an isolated composition, which does not contain intact cells. Examples of cell free compositions include cell extracts and compositions containing isolated proteins.

As used herein, the term “reporter gene” refers to any gene that expresses a detectable gene product, e.g., RNA or protein. As used herein the term “reporter protein” refers to a protein encoded by a reporter gene. Preferred reporter genes are those that are readily detectable. The reporter gene can also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898), Enhanced Green Fluorescent Proteins (EGFP), Discosoma sp. red fluorescent protein (dsRED).

As used herein, the term “XBP-1-responsive element” refers to a DNA sequence that is directly or indirectly regulated by the activity of the XBP-1 (whereby activity of XBP-1 can be monitored, for example, via transcription of a reporter gene).

As used herein, the term “cells deficient in XBP-1” includes cells of a subject that are naturally deficient in XBP-1, as wells as cells of a non-human XBP-1 deficient animal, e.g., a mouse, that have been altered such that they are deficient in XBP-1. The term “cells deficient in XBP-1” is also intended to include cells isolated from a non-human XBP-1 deficient animal or a subject that are cultured in vitro.

As used herein, the term “non-human XBP-1 deficient animal” refers to a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal, such that the endogenous XBP-1 gene is altered, thereby leading to either no production of XBP-1 or production of a mutant form of XBP-1 having deficient XBP-1 activity. Preferably, the activity of XBP-1 is entirely blocked, although partial inhibition of XBP-1 activity in the animal is also encompassed. The term “non-human XBP-1 deficient animal” is also intended to encompass chimeric animals (e.g., mice) produced using a blastocyst complementation system, such as the RAG-2 blastocyst complementation system, in which a particular organ or organs (e.g., the lymphoid organs) arise from embryonic stem (ES) cells with homozygous mutations of the XBP-1 gene. The term “non-human XBP-1 deficient animal” is also intended to encompass animals (e.g., mice) that contain a conditional allele(s) of the XBP-1 gene, such as a cre-lox containing animal in which the XBP-1 gene is rendered non-functional following, e.g., mating of an animal containing a floxed allele with an animal containing a cre allele (cre recombinase, e.g., under the control of the M×1 promoter), such as those described in, e.g., Lee, et al. (Science. 2008 Jun. 13; 320(5882):1492-6) or, Hetz, et al. (2008) Proc Natl Acad Sci, USA 105:757, the contents of each of which are incorporated herein by reference.

As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

In one embodiment, a nucleic acid molecule of the invention mediates RNAi. In one embodiment, a nucleic acid molecule of the invention is a “short interfering RNA” (“siRNA”) molecule. In another embodiment, a nucleic acid molecule of the invention is “small hairpin RNA” (“shRNA”) molecule. In another embodiment, the nucleic acid molecule of the invention is a “microRNA” (“miRNA”) molecule. As used herein, siRNA, shRNA and miRNA molecules are collectively referred to as “RNAi agents”.

RNA interference (RNAi) is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell TR, and Doering TL. 2003. Trends Microbiol. 11:37-43; Bushman F. 2003. Mol Therapy. 7:9-10; McManus MT and Sharp PA. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs or Ambion. In one embodiment one or more of the chemistries described herein or known in the art for use in antisense RNA can be employed in molecules that mediate RNAi.

As used herein, the term “dominant negative” includes molecules, such as XBP-1 molecules (e.g., portions or variants thereof) that compete with native (i.e., wild-type) XBP-1 molecules, but which do not have XBP-1 activity. Such molecules effectively decrease XBP-1 activity in a cell.

In one embodiment, small molecules can be used as test compounds. The term “small molecule” is a term of the art and includes molecules that are less than about 7500, less than about 5000, less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., Cane et al. 1998. Science 282:63), and natural product extract libraries. In another embodiment, the compounds are small, organic non-peptidic compounds. In a further embodiment, a small molecule is not biosynthetic. For example, a small molecule is preferably not itself the product of transcription or translation.

Various aspects of the present invention are described in further detail in the following subsections.

### II. SCREENING ASSAYS

In one aspect, the invention provides methods (also referred to herein as “screening assays”) for identifying agents for treating, preventing, and/or delaying the onset of (e.g., modulating at least one symptom of) a neurodegenerative disorder associated with protein aggregation, i.e., candidate or test compounds or agents (e.g., enzymes, peptides, peptidomimetics, small molecules, ribozymes, or antisense molecules, or RNAi agents) which bind, e.g., to XBP-1, IRE-1 alpha, and/or EDEM; and/or have an inhibitory effect on the expression or activity of XBP-1, IRE-1 alpha, and/or EDEM.

In one embodiment, the ability of a compound to directly inhibit the expression or activity of XBP-1, IRE-1 alpha, and/or EDEM is measured in a screening assay of the invention.

The indicator composition can be a cell that expresses the XBP-1, IRE-1 alpha, and/or EDEM protein, for example, a cell that naturally expresses or, more preferably, a cell that has been engineered to express the protein(s) by introducing into the cell an expression vector encoding the protein(s). Preferably, the cell is a mammalian cell, e.g., a human cell. In another embodiment, the cell is a motor neuron cell. Alternatively, the indicator composition can be a cell-free composition that includes the protein (e.g., a cell extract or a composition that includes e.g., either purified natural or recombinant protein). In one embodiment, the cell is under ER stress. In one embodiment, the cell is undergoing nutrient starvation.

Compounds identified as modulating, e.g., downmodulating the expression, activity, and/or stability of spliced XBP-1 and/or the expression and/or activity of IRE-1 alpha and/or the expression and/or activity of EDEM using the assays described herein are useful for decreasing aberrant protein aggregation associated with a neurodegenerative disease, and/or decreasing apoptosis of motor neuron cells, and/or preventing and/or treating and/or delaying the onset of a neurodegenerative disorder, associated with protein aggregation, e.g., at least one symptom associate with the disease.

Exemplary diseases(s) that can benefit from downmodulation include, for example, Amyotrophic Lateral Sclerosis (ALS), Huntington's Disease (HD), Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCAT), spinocerebellar ataxia type 17 (SCA17), chronic liver diseases, familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, or subacute sclerosing panencephalitis.

Exemplary symptoms associated with a neurodegenerative disorder associated with protein aggregation that can benefit from modulation, e.g., downmodulation, of the expression and/or activity of XBP-1, IRE-1 alpha, and/or EDEM include, for example twitching, cramping, or stiffness of affected muscles, muscle weakness affecting an arm or a leg; slurred and nasal speech, difficulty moving, swallowing (dysphagia), chewing and speaking or forming words (dysarthria), tight and stiff muscles (spasticity) and exaggerated reflexes (hyperreflexia) including an overactive gag reflex, abnormal Babinski's sign, muscle atrophy, muscle cramps, pseudobulbar affect, also known as “emotional lability”, maintainging weight, anxiousness, depression, frontotemporal dementia, mild problems with word-generation, attention, or decision-making, poor breathing at night (nocturnal hypoventilation), clumsiness, haw clenching (bruxism), loss of coordination and balance, uncontrolled continual muscular contractions (dystonia), etc.

The subject screening assays can be performed in the presence or absence of other agents. In one embodiment, the subject assays are performed in the presence of an agent that affects the unfolded protein response, e.g., tunicamycin, which evokes the UPR by inhibiting N-glycosylation, or thapsigargin. In another embodiment, the subject assays are performed in the presence of an agent that inhibits autophagy, e.g., 3-methyladenine, Wormannin, antisense Beclin-1 (ATG6) nucleic acid molecules. In another embodiment, the subject assays are performed in the presence of an agent that inhibits degradation of proteins by the ubiquitin-proteasome pathway (e.g., peptide aldehydes, such as MG132). In another embodiment, the screening assays can be performed in the presence or absence of a molecule that enhances cell activation.

In another aspect, the invention pertains to a combination of two or more of the assays described herein. For example, a modulating agent can be identified using a cell-based or a cell-free assay, and the ability of the agent to modulate the activity of XBP-1 can be confirmed in vivo, e.g., in an animal model of neurodegenerative disease associated with aberrant protein aggregation.

In addition, a modulating agent can be identified using a cell-based or a cell-free assay, and the ability of the agent to modulate, e.g., autophagy, motor neuron survival, aberrant protein aggregation associated with a neurodegenerative disease, aberrant protein aggregate detergent insolubility, apotptosis, etc., can be subsequently determined.

Moreover, a modulator of XBP-1, IRE-1 alpha, and/or EDEM, identified as described herein (e.g., an enzyme, an antisense nucleic acid molecule, or a specific antibody, or a small molecule) can be used in an animal model to determine the efficacy, toxicity, or side effects of treatment with such a modulator. Alternatively, a modulator identified as described herein can be used in an animal model to determine the mechanism of action of such a modulator.

In another embodiment, it will be understood that similar screening assays can be used to identify compounds that indirectly modulate the activity and/or expression of XBP-1, IRE-1 alpha, and/or EDEM, e.g., by performing screening assays such as those described above using molecules with which XBP-1, IRE-1 alpha, and/or EDEM interact, e.g., molecules that act either upstream or downstream of XBP-1, IRE-1 alpha, and/or EDEM in a signal transduction pathway.

The cell based and cell free assays of the invention are described in more detail below.

A. Cell Based Assays

The indicator compositions of the invention can be a cell that expresses an XBP-1, IRE-1 alpha, and/or EDEM polypeptide, for example, a cell that naturally expresses endogenous XBP-1 or, more preferably, a cell that has been engineered to express an exogenous XBP-1 protein by introducing into the cell an expression vector encoding the protein. Alternatively, the indicator composition can be a cell-free composition that includes XBP-1 or a non-XBP-1 protein, such as IRE-1 alpha, or a composition that includes purified XBP-1, IRE-1 alpha, and/or EDEM. In one embodiment, cells deficient in XBP-1, IRE-1 alpha, and/or EDEM may be used in the methods of the invention. In one embodiment, such cells are isolated from a non-human animal deficient in XBP-1, IRE-1 alpha, and/or EDEM. In another embodiment, such cells are produced by contacting a cell that naturally expresses endogenous, XBP-1, IRE-1 alpha, and/or EDEM with an agent, e.g., an antisenseXBP-1, IRE-1 alpha, and/or EDEM nucleic acid molecule, to downregulate the expression of XBP-1, IRE-1 alpha, and/or EDEM.

In another embodiment of the invention, cells suitable for use in the assays of the invention may comprise an expression vector comprising a nucleic acid molecule encoding a protein that when mutated forms protein aggregates that are associated with a neurodenerative disease, e.g., SOD1, e.g., SOD1^{G93A}, SOD1^{G86R}, polyglutamine, e.g., (poly (CAG)), e.g., Q, e.g., poly(Q)₁₁, poly(Q)₇₂.

Compounds that modulate expression and/or activity of XBP-1, IRE-1 alpha, and/or EDEM can be identified using various “read-outs.”

For example, an indicator cell can be transfected with an XBP-1 expression vector, incubated in the presence and in the absence of a test compound, and the effect of the compound on the expression of the molecule or on a biological response regulated by XBP-1 can be determined. In one embodiment, unspliced XBP-1 (e.g., capable of being spliced so that the cell will make both forms, or incapable of being spliced so the cell will make only the unspliced form) can be expressed in a cell. In another embodiment, spliced XBP-1 can be expressed in a cell. The biological activities of XBP-1 include activities determined in vivo, or in vitro, according to standard techniques. An XBP-1 activity can be a direct activity, such as an association with an XBP-1-target molecule (e.g., a nucleic acid molecule to which XBP-1 binds such as the transcriptional regulatory region of a chaperone gene) or a protein such as the IRE-1 alpha protein. Alternatively, an XBP-1 activity is an indirect activity, such as a cellular signaling activity or alteration in gene expression occurring downstream of the interaction of the XBP-1 protein with an XBP-1 target molecule or a biological effect occurring as a result of the signaling cascade triggered by that interaction. For example, biological activities of XBP-1 described herein include: modulation of a neurodegenerative diease associated with protein aggregation, modulation of autophagy, modulation of motor neuron survival, modulation of the generation of protein aggregation associated with a neurodegenerative disease, modulation of apoptosis, modulation of the UPR, modulation of the proteasome pathway, and modulation of protein folding and transport.

The biological activities of IRE-1 alpha include activities determined in vivo, or in vitro, according to standard techniques. An IRE-1 alpha activity can be a direct activity, such as an association with an IRE-1 alpha-target molecule (e.g., a nucleic acid molecule to which IRE-1 alpha binds or a protein such as the XBP-1 protein), and/or autophosphorylation. Alternatively, an IRE-1 alpha activity is an indirect activity, such as a cellular signaling activity or alteration in gene expression occurring downstream of the interaction of the IRE-1 alpha protein with an IRE-1 alpha target molecule or a biological effect occurring as a result of the signaling cascade triggered by that interaction. For example, biological activities of IRE-1 alpha described herein include: modulation of a neurodegenerative diease associated with protein aggregates, modulation of autophagy, modulation of motor neuron survival, modulation of the generation of protein aggregation associated with a neurodegenerative disease, modulation of apoptosis, modulation of the UPR, modulation of the proteasome pathway, and modulation of protein folding and transport.

The biological activities of EDEM include activities determined in vivo, or in vitro, according to standard techniques. An EDEM activity can be a direct activity, such as an association with an EDEM-target molecule (e.g., a nucleic acid molecule to which EDEM binds or a protein such as the calnexin protein. Alternatively, an EDEM activity is an indirect activity, such as a cellular signaling activity or alteration in gene expression occurring downstream of the interaction of the EDEM protein with an EDEM-target molecule or a biological effect occurring as a result of the signaling cascade triggered by that interaction. For example, biological activities of EDEM described herein include: modulation of a neurodegenerative diease associated with protein aggregates, modulation of autophagy, modulation of motor neuron survival, modulation of the generation of protein aggregation associated with a neurodegenerative disease, modulation of apoptosis, modulation of the UPR, modulation of ERAD, modulation of cellular protein ubiquitination, modulation of the proteasome pathway, and modulation of protein folding and transport.

To determine whether a test compound modulates XBP-1, IRE-1 alpha, and/or EDEM protein expression, in vitro transcriptional assays can be performed. In one example of such an assay, the full length XBP-1 gene or promoter and enhancer of XBP-1 operably linked to a reporter gene such as chloramphenicol acetyltransferase (CAT) or luciferase and introduced into host cells. The expression or activity of XBP-1 or the reporter gene can be measured using techniques known in the art. The ability of a test compound to regulate the expression or activity of a molecule in a signal transduction pathway involving XBP-1 can be similarly tested.

As used interchangeably herein, the terms “operably linked” and “operatively linked” are intended to mean that the nucleotide sequence is linked to a regulatory sequence in a manner which allows expression of the nucleotide sequence in a host cell (or by a cell extract).

In another embodiment, modulation of expression of a protein whose expression is regulated by XBP-1 is measured. Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of protein desired to be expressed.

Exemplary constructs can include, for example, an XBP-1 target sequence TGGATGACGTGTACA fused to the minimal promoter of the mouse RANTES gene (Clauss et al. Nucleic Acids Research 1996. 24:1855) or the ATF6/XBP-1 target TCGAGACAGGTGCTGACGTGGCGATTCC and comprising −53/+45 of the cfos promoter (J. Biol. Chem. 275:27013) fused to a reporter gene. In one embodiment, multiple copies of the XBP-1 target sequence can be included.

A variety of reporter genes are known in the art and are suitable for use in the screening assays of the invention. Examples of suitable reporter genes include those which encode chloramphenicol acetyltransferase, beta-galactosidase, alkaline phosphatase or luciferase, green fluorescent protein, red fluorescent protein. Standard methods for measuring the activity of these gene products are known in the art.

A variety of cell types are suitable for use as an indicator cell in the screening assay. Preferably a cell line is used which expresses low levels of endogenous XBP-1, IRE-1 alpha, and/or EDEM, and is then engineered to express recombinant XBP-1, IRE-1 alpha, and/or EDEM. Cells for use in the subject assays include both eukaryotic and prokaryotic cells. For example, in one embodiment, a cell is a bacterial cell. In another embodiment, a cell is a fungal cell, such as a yeast cell. In another embodiment, a cell is a vertebrate cell, e.g., an avian cell or a mammalian cell (e.g., a murine cell, or a human cell).

In one embodiment, the level of expression of the reporter gene in the indicator cell in the presence of the test compound is higher than the level of expression of the reporter gene in the indicator cell in the absence of the test compound and the test compound is identified as a compound that stimulates the expression of the molecule. In another embodiment, the level of expression of the reporter gene in the indicator cell in the presence of the test compound is lower than the level of expression of the reporter gene in the indicator cell in the absence of the test compound and the test compound is identified as a compound that inhibits the expression of the molecule.

In another embodiment, the level of expression of genes whose expression is regulated by XBP-1, e.g., chaperone genes, e.g., ERdj4, p58^{IPK}, EDEM, PDI-P5, RAMP4, HEDJ, BiP, Armet, DNAJB9, CHOP, ATF6α, Gpr58, Sec61, Wfs-1, Herp, XBP-1 and can be measured using standard techniques. The sequences of such genes are known in the art.

The nucleotide and amino acid sequence of human Erdj4 may be found in, for example, GenBank accession number: gi:9558754; The nucleotide and amino acid sequence of murine Erdj4 may be found in, for example, GenBank accession number: gi:142365683.

The nucleotide and amino acid sequence of human Gpr58 may be found in, for example, GenBank accession number: gi:67083697; the nucleotide and amino acid sequence of murine Gpr58 may be found in, for example, GenBank accession number: gi:112293263.

The nucleotide and amino acid sequence of human Sec61 (Sec61 alpha 1 subunit) may be found in, for example, GenBank accession number: gi:60218911; The nucleotide and amino acid sequence of murine Sec61 (Sec61 alpha 1 subunit) may be found in, for example, GenBank accession number: gi:146134405. The nucleotide and amino acid sequence of human Sec61 (Sec61 alpha 2 subunit) may be found in, for example, GenBank accession number: gi:14589846; The nucleotide and amino acid sequence of murine Sec61 (Sec61 alpha 2 subunit) may be found in, for example, GenBank accession number: gi:118130500. The nucleotide and amino acid sequence of human Sec61 gamma subunit (SEC61G), transcript variant 2 may be found in, for example, GenBank accession number: gi:60279264; The nucleotide and amino acid sequence of human Sec61 gamma subunit (SEC61G), transcript variant 1 may be found in, for example, GenBank accession number: gi:60279263; The nucleotide and amino acid sequence of murine Sec61 gamma subunit (SEC61G) may be found in, for example, GenBank accession number: gi:149233905.

The nucleotide and amino acid sequence of human Wfs-1 may be found in, for example, GenBank accession number: gi:13376995; The nucleotide and amino acid sequence of murine Wfs-1 may be found in, for example, GenBank accession number: gi:119672922.

The nucleotide and amino acid sequence of human herp (homocysteine-inducible, endoplasmic reticulum stress-inducible, ubiquitin-like domain member 1 (HERPUD1), transcript variant 2 may be found in, for example, GenBank accession number: gi:58530856. The nucleotide and amino acid sequence of human herp (homocysteine-inducible, endoplasmic reticulum stress-inducible, ubiquitin-like domain member 1 (HERPUD1), transcript variant 3 may be found in, for example, GenBank accession number: gi:58530858. The nucleotide and amino acid sequence of human herp (homocysteine-inducible, endoplasmic reticulum stress-inducible, ubiquitin-like domain member 1 (HERPUD1), transcript variant 1 may be found in, for example, GenBank accession number: gi:58530855. The nucleotide and amino acid sequence of murine herp (homocysteine-inducible, endoplasmic reticulum stress-inducible, ubiquitin-like domain member 1 (HERPUD1), may be found in, for example, GenBank accession number: gi:11612514.

The nucleotide and amino acid sequence of human BiP/GRP78 may be found in, for example, GenBank accession number: gi:21361242; The nucleotide and amino acid sequence of murine GRP78 may be found in, for example, GenBank accession number: gi:31981721, gi: 1143491.

The nucleotide and amino acid sequence of human p58ipk may be found in, for example, GenBank accession number: gi:24234721. The nucleotide and amino acid sequence of murine p58ipk may be found in, for example, GenBank accession number: gi:31542562. The nucleotide and amino acid sequence of rat p58ipk may be found in, for example, GenBank accession number: gi:11560029.

The nucleotide and amino acid sequence of human RAMP4 may be found in, for example, GenBank accession number: gi:109809760. The nucleotide and amino acid sequence of murine RAMP4 may be found in, for example, GenBank accession number: gi:141801749. The nucleotide and amino acid sequence of rat RAMP4 may be found in, for example, GenBank accession number: gi:42476131.

The nucleotide and amino acid sequence of human HEDJ may be found in, for example, GenBank accession number: gi:51317390. The nucleotide and amino acid sequence of murine HEDJ may be found in, for example, GenBank accession number: gi:142376452. The nucleotide and amino acid sequence of rat HEDJ may be found in, for example, GenBank accession number: gi:62543490.

The nucleotide and amino acid sequence of human Armet may be found in, for example, GenBank accession number: gi:54873599. The nucleotide and amino acid sequence of murine Armet may be found in, for example, GenBank accession number: gi:142365315. The nucleotide and amino acid sequence of rat Armet may be found in, for example, GenBank accession number: gi:157823810.

The nucleotide and amino acid sequence of human DNAJB9 may be found in, for example, GenBank accession number: gi:9558754. The nucleotide and amino acid sequence of murine DNAJB9 may be found in, for example, GenBank accession number: gi:142365683. The nucleotide and amino acid sequence of rat DNAJB9 may be found in, for example, GenBank accession number: gi:10732860.

The nucleotide and amino acid sequence of human ATF6α may be found in, for example, GenBank accession number: gi:56786156. The nucleotide and amino acid sequence of murine ATF6α may be found in, for example, GenBank accession number: gi:124486810. The nucleotide and amino acid sequence of rat ATF6α may be found in, for example, GenBank accession number: gi:157821878.

Also, see, e.g., p58^{ipk} (e.g., XM_{—}209778 [gi:2749842] or NM_{—}006260 [gi:24234721]), EDEM (e.g., NM_{—}014674 [gi:7662001]), PDI-P5 (e.g., D49489 [gi: 1136742]), RAMP4 (e.g., AF136975 [gi:12239332]), HEDJ (e.g., AF228505 [gi: 7385134]), ATF6q, e.g., NM_{—}007348 [gi:6671584], XBP-1 (e.g., NM_{—}005080 [gi:14110394]), Armet (e.g., NM_{—}006010 [gi:51743920]), DNAJB9 (which encodes mDj7) e.g., (NM_{—}012328 [gi:9558754]),

XBP-1 (e.g., A36299 [gi:105867], NP_{—}005071 [gi:4827058], P17861 [gi:139787], CAA39149 [gi:287645], and BAA82600 [gi:5596360], AF027963 [gi: 13752783]; NM_{—}013842 [gi:13775155]; or M31627 [gi:184485], NM_{—}005080 [gi:14110394] or NM_{—}013842 [gi:13775155]), the MHC class II genes (various MHC class II gene sequences are known in the art) and the IL-6 gene (e.g., MN_{—}000600 [gi:10834983]).

In one embodiment, the invention provides methods for identifying compounds that modulate cellular responses in which XBP-1, IRE-1 alpha, and/or EDEM are involved.

For example, in one embodiment, modulation of autophagy can be determined and used as an indicator of modulation of XBP-1, IRE-1 alpha, and/or EDEM activity. For example, the generation and/or quantification of autophagosomes may be determined using transmission electron microscopy and/or confocal microscopy. Autophagosomes can be recognized as being double-membrane-enclosed vesicles, which contain undigested cytoplasmic material including organelles, and which are dramatically reduced by known inhibitors of autophagy such as 3 methyladenine (3-MA, amino acid, or an antisense nucleic acid molecule to ATG (Autophagy) genes (ATG5, ATG9). Autolysosomes are single-membrane vesicles which may still contain some cytoplasmic components at various stages of degradation. Their formation is inhibited by V-ATPase specific inhibitor, such as bafilomycin Al (BafAl). Commercially available antibodies to ATG proteins are available and may be used to stain and visualize cellular structures associated with autophagy.

Cells may also be transiently transfected with an expression vector comprising an art recognized marker of autophagy that specifically localizes to autophagosomes in order to determine whether a test compound modulates autophagy. In one embodiment, a marker of autohpagy is an ATG protein, e.g., ATG8 (also known as LC3 and microtubule-associated protein 1 light chain 3 alpha). In one embodiment the ATG8 protein is a fusion protein, e.g., a nucleic acid molecule encoding the ATG8 protein is operably linked to a non-ATG8 protein, e.g., a detectable marker, to allow for visualization of the ATG8 protein expressed by the vector, e.g., dsRED, EGFP (Kabeya, et al. 2000. EMBO J 19; 5720). The nucleotide and amino acid sequence of human ATG8 is known and can be found in, for example, GenBank accession No. gi:31563519 (human transcript variant 1) and gi:31563517 (human transcript variant 2), gi:23956147 (mouse); gi:41054833 (rat).

Furthermore, two forms of LC3 (ATG8) have been described: LC3-I and LC3-II, and it has been demonstrated that upon autophagy induction, the cytosolic LC3-I is processed to LC3-II which specifically associates with autophagosome membranes. On SDS-PAGE, LC3-II (apparent mobility, 16 kD) migrates faster than LC3-I (apparent mobility, 18 kD). Consequently, measurement of LC3-II levels by immunoblotting is another method for determining the autophagic activity of mammalian cells. The expression of LC3 may also be determined using, for example, RT-PCR.

In addition, LAMP1 and LAMP2 have been shown to be specific markers of lysosomes (autolysosomes) and cells may be stained with commercially available antibodies to visualize these cellular structures to determine whether a test compound modulates autophagy as well as to determine the effect of the compound on lysosome content.

In another embodiment, the ability of a compound to modulate apoptosis (and/or modulate cell viability and/or survival) can be determined. In one embodiment, the ability of a compound to modulate apoptosis in a cell or a cell under ER stress is determined. Apoptosis can be measured in the presence or the absence of Fas-mediated signals. In one embodiment, cytochrome C release from mitochondria during cell apoptosis can be detected, e.g., plasma cell apoptosis (as described in, for example, Bossy-Wetzel E. et al. (2000) Methods in Enzymol. 322:235-42). Other exemplary assays include: cytofluorometric quantitation of nuclear apoptosis induced in a cell-free system (as described in, for example, Lorenzo H. K. et al. (2000) Methods in Enzymol. 322:198-201); apoptotic nuclease assays (as described in, for example, Hughes F. M. (2000) Methods in Enzymol. 322:47-62); analysis of apoptotic cells, e.g., apoptotic plasma cells, by flow and laser scanning cytometry (as described in, for example, Darzynkiewicz Z. et al. (2000) Methods in Enzymol. 322:18-39); detection of apoptosis by annexin V labeling (as described in, for example, Bossy-Wetzel E. et al. (2000) Methods in Enzymol. 322:15-18); transient transfection assays for cell death genes (as described in, for example, Miura M. et al. (2000) Methods in Enzymol. 322:480-92); and assays that detect DNA cleavage in apoptotic cells, e.g., apoptotic plasma cells (as described in, for example, Kauffman S. H. et al. (2000)Methods in Enzymol. 322:3-15). Apoptosis can also be measured by propidium iodide staining or by TUNEL assay. In another embodiment, the transcription of genes associated with a cell signaling pathway involved in apoptosis (e.g., JNK) can be detected using standard methods.

In one embodiment, cell viability is determined using the art-known MTT colorimetric assay. In another embodiment, cell viability or apoptosis of cells is determined using the art known propidium iodide assay (see, e.g., Hetz et al. (2006) Science 312:572).

In another embodiment, motor neuron survival is determined in, e.g., an animal model of a neurodegenerative disease associated with aberrant protein aggregation. For example, a test agent may be administed to the non-human animal model and tissue samples, e.g., neural, spinal cord tissue, may be collected and processed using standard histological techniques. For example, apoptotic cells may be visualized as described above, using, for example the TUNEL assay and motor neuron cells may be visualized using an anti-cholinesterase (antiChAT) and/or anti-NeuN antibody. In addition, autophagosomes and lysosmes may be visualized in the cells suing anti-ATG antibodies and anti-LAMP2 antibodies as described above. Such stained tissue samples may be analyzed using, for example confocal microscopy.

In another embodiment, the formation and quantification of protein aggregates, e.g., high molecular weight and detergent insoluble aggregates, and intracellular inclusions of protein aggregates associated with a neurogegenrative disease may be determined in vitro or in a non-human animal model of a neurodegenerative disease associated with protein aggregation. In vitro methods generally involve the use of an expression vector comprising a nucleic acid molecule encoding a protein that, when mutated, forms aggregates of the protein and intracellular inclusions of such aggregates. In one embodiment, the expression vector further comprises a nucleic acid molecule encoding a heterologous polypeptide, e.g., a detectable marker, e.g., EGFP. Cells may be transiently transfected with the expression vector and intracellular inclusions of the protein may be visualized using, for example, fluorescent confocal microscopy. Alternatively, cell lysates from such transiently transfected cell may be used for Western blot analysis, e.g., by staining using an antibody specific to the protein that aggregates. Such lysates may be prepared in a detergent, such as NP-40, prior to Western blot analysis to assay for detergent soluble and insoluble protein.

For example, in one embodiment, an assay using the transient expression of a protein that can form aggregates e.g., human SOD1^{WT} and the mutants SOD1^{G93A} and SOD1^{G85R} are made as fusion proteins, e.g., EGFP fusion proteins. These constructs can be employed to visualize and quantify the formation of intracellular SOD1 inclusions in living cells by fluorescent confocal microscopy. SOD1 oligomers were visualized in total cell extracts prepared in RIPA buffer and sonication, and then analyzed by Western blot. Alternatively, nuclear cell lysates can be prepared in 1% NP-40 in PBS containing protease inhibitors. After solubilization on ice for 30 min, cell nuclei are precipitated by centrifugation at 3000 rpm for 5 min and cell extracts arecentrifuged at 10,000 g for 10 min to collect NP-40 soluble and insoluble material. Pellets are resuspended in Western blot sampler buffer containing SDS.

In another embodiment, assays following the recommendations and precautions described in (Klionsky et al., 2008) can be used. Lysosomes or acidic compartments can be visualized after staining with different dyes. For example, living cells can be stained with 200 nM lysotraker or 600 nM Acridine orange for 45 min at 37° C. and 5% CO₂. Alternatively, cells can be loaded with DQ-BSA to monitor lysosomal activity as previously described (Klionsky et al., 2008). Autophagy may be monitored by analyzing LC3-positive dots or the levels of LC3-II by Western blot and its flux through the autophagosomal/lysosomal pathway as described (Klionsky et al., 2008). To follow the flow of LC3I/II through the autophagy pathway, cells can be treated with a mix of 200 nM bafilomycin A₁, 10 μg/mlpepstatin and 10 μg/ml E64d. Alternatively, to monitor flux transient expression of a tandem monomeric RFP-GFP-tagged LC3 (Klionsky et al., 2008) molecule can be used.

Methods to monitor the formation and characterize and quantify protein aggregates and intracellular inclusions of protein aggregates associated with a neurogegenrative disease in a non-human animal model of a neurodegenerative disease associated with protein aggregation generally involve collection and processing of tissue samples, e.g., neural, spinal cord tissue, using standard histological techniques. Cells may be examined microscopically and/or lysed for Western blot analysis as described above.

Suitable non-human animal models of neurodegenerative disease associated with protein aggregation are known in the art and are commercially available from, for example, Jackson Laboratory. In one embodiment, a non-human animal model of neurodegenerative disease associated with protein aggregartion is a model of ALS. In one embodiment, the model of ALS is a transgenic mouse expressing human SOD1^{G93A}. In another embodiment, the model of ALS is a transgenic mouse expressing mouse SOD1^{G86R}. In one embodiment a non-human animal model of neurodegenerative disease associated with protein aggregartion is a model of HD. In one embodiment, the model of HD is a transgenic mouse expressing the 5′ end of the human Huntington gene carrying (CAG)115-(CAG)150 repeat expansions.

In another embodiment, modulation of the UPR or ER stress can also be determined and used as an indicator of modulation of XBP-1, IRE-1 alpha, and/or EDEM activity. Transcription of genes encoding molecular chaperones and folding enzymes in the endoplasmic reticulum (ER) is induced by accumulation of unfolded proteins in the ER. This intracellular signaling, known as the unfolded protein response (UPR), is mediated by the cis-acting ER stress response element (ERSE) or unfolded protein response element (UPRE) in mammals.

The activation of the kinase PERK can also be measured to determine whether an agent moduleates ER stress by measuring the induction of CHOP

In another embodiment, modulation of ERAD may be measured by determining the basal amount of ubiquitinated cellular protein, e.g., the amount of high molecular weight ubiquitinated protein in a cell, as compared to an appropriate control.

The processing of ATF6 alpha can also be measured to determine whether an agent modulates ER stress. The basic leucine zipper protein ATF6 alpha isolated as a CCACG-binding protein is synthesized as a transmembrane protein in the ER, and ER stress-induced proteolysis produces a soluble form of ATF6 alpha that translocates into the nucleus.

In another embodiment, the expression of molecular chaperones such as CHOP, GRP78 or BIP can be measured.

Modulation of XBP-1, IRE-1 alpha, and/or EDEM activity (modulation of UPR) can also be measured by, for example, measuring the changes in the endogenous levels of mRNA and the transcription or production of proteins such as ERdj4, p58^{ipk}, EDEM, PDI-P5, RAMP4, HEDJ, BiP, ATF6α, XBP-1, Armet and DNAJB9, GRP58, Sec61, wfs-1, herp, CHOP, or folding catalysts using routine ELISA, Northern and Western blotting techniques. In addition, the attenuation of translation associated with the UPR can be measured, e.g., by measuring protein production (Ruegsegger et al. 2001. Cell 107:103). Preferred proteins for detection are expressed on the cell surface or are secreted.

In another embodiment, the phosphorylation of IRE-1 alpha, PERK and/or eukaryotic initiation factor 2 (eIF2a) can be measured. In another embodiment, the accumulation of aggregated, misfolded, or damaged proteins in a cell can be monitored (Welch, W. J. 1992 Physiol. Rev. 72:1063; Gething and Sambrook. 1992. Nature. 355:33; Kuznetsov et al. 1997. J. Biol. Chem. 272:3057), as can the amount of total cellular ubiquitinated proteinas an indicator of ERAD function.

In one embodiment, modulation of XBP-1 activity can be measured by determining the phosphorylation status of IRE-1 alpha, PERK or eIF2α e.g., using commercially available antibodies that specifically recognize phosphorylated forms of the proteins. Increased phosphorylation of these molecules is observed under conditions of ER stress.

In another embodiment, the ability of a compound to modulate the proteasome pathway of a cell can be determined using any of a number of art-recognized techniques. For example, in one embodiment, the half life of normally short-lived regulatory proteins (e.g., NF-kB, cyclins, oncogenic products or tumor suppressors) can be measured to measure the degradation capacity of the proteasome. In another embodiment, the presentation of antigen in the context of MHC molecules on the surface of cells can be measured (e.g., in an in vitro assay of T cell activation) as proteasome degradation of antigen is important in antigen processing and presentation. In another embodiment, threonine protease activity associated with the proteasome can be measured. Agents that modulate the proteasome pathway will affect the normal degradation of these proteins. In another embodiment, the modulation of the proteasome pathway can be measured indirectly by measuring the ratio of spliced to unspliced XBP-1 or the ratio of unspliced to spliced XBP-1. Inhibition of the proteasome pathway, e.g., by the inhibitor MG-132, leads to an increase in the level of unspliced XBP-1 as compared to spliced XBP-1.

The techniques for assessing the ratios of unspliced to spliced XBP-1 and spliced to unspliced XBP-1 are routine in the art. For example, the two forms can be distinguished based on their size, e.g., using northern blots or western blots. Because the spliced form of XBP-1 comprises an exon not found in the unspliced form, in another embodiment, antibodies that specifically recognize the spliced or unspliced form of XBP-1 can be developed using techniques well known in the art (Yoshida et al. 2001. Cell. 107:881). In addition, PCR can be used to distinguish spliced from unspliced XBP-1. For example, as described herein, primer sets can be used to amplify XBP-1 where the primers are derived from positions 410 and 580 of murine XBP-1, or corresponding positions in related XBP-1 molecules, in order to amplify the region that encompasses the splice junction. A fragment of 171 base pairs corresponds to unspliced XBP-1 mRNA. An additional band of 145 bp corresponds to the spliced form of XBP-1. The ratio of the different forms of XBP-1 can be determined using these or other art recognized methods.

Compounds that alter the ratio of unspliced to spliced XBP-1 or spliced to unspliced XBP-1 can be useful to modulate the biological activities of XBP-1, and the levels of these different forms of XBP-1 can be measured using various techniques described above, or known in the art, and a ratio determined.

In one embodiment, the ability of a compound to modulate protein folding or transport can be determined. The expression of a protein on the surface of a cell or the secretion of a secreted protein can be measured as indicators of protein folding and transport. Protein expression on a cell can be measured, e.g., using FACS analysis, surface iodination, immunoprecipitation from membrane preparations. Protein secretion can be measured, for example, by measuring the level of protein in a supernatant from cultured cells. The production of any secreted protein can be measured in this manner. The protein to be measured can be endogenous or exogenous to the cell. In preferred embodiment, the protein is selected from the group consisting of: α-fetoprotein, α-antitrypsin, albumin, luciferase and immunoglobulins. The production of proteins can be measured using standard techniques in the art.

In yet another embodiment, the ability of a compound to modulate translocation of spliced XBP-1 to the nucleus can be determined. Translocation of spliced XBP-1 to the nucleus can be measured, e.g., by nuclear translocation assays in which the emission of two or more fluorescently-labeled species is detected simultaneously. For example, the cell nucleus can be labeled with a known fluorophore specific for DNA, such as Hoechst 33342. The spliced XBP-1 protein can be labeled by a variety of methods, including expression as a fusion with GFP or contacting the sample with a fluorescently-labeled antibody specific spliced XBP-1. The amount spliced XBP-1 that translocates to the nucleus can be determined by determining the amount of a first fluorescently-labeled species, i.e., the nucleus, that is distributed in a correlated or anti-correlated manner with respect to a second fluorescently-labeled species, i.e., spliced XBP-1, as described in U.S. Pat. No. 6,400,487, the contents of which are hereby incorporated by reference.

In another embodiment, the ability of XBP-1, IRE-1 alpha, and/or EDEM to be acted on by an enzyme or to act on a substrate can be measured. For example, in one embodiment, the effect of a compound on the phosphorylation of IRE-1 alpha, the ability of IRE-1 alpha to process XBP-1, the ability of PERK to phosphorylate a substrate can be measured using techniques that are known in the art.

The ability of the test compound to modulate XBP-1, IRE-1 alpha, and/or EDEM binding to a substrate or target molecule can also be determined. Determining the ability of the test compound to modulate XBP-1 (or IRE-1 alpha) binding to a target molecule (e.g., a binding partner such as a substrate) can be accomplished, for example, by coupling the target molecule with a radioisotope or enzymatic label such that binding of the target molecule to XBP-1 can be determined by detecting the labeled XBP-1 (or IRE-1 alpha target molecule in a complex. Alternatively, XBP-1 could be coupled with a radioisotope or enzymatic label to monitor the ability of a test compound to modulate XBP-1 binding to a target molecule in a complex. Determining the ability of the test compound to bind to XBP-1 can be accomplished, for example, by coupling the compound with a radioisotope or enzymatic label such that binding of the compound to XBP-1 can be determined by detecting the labeled compound in a complex. For example, targets can be labeled with ¹²⁵I, ³⁵S, ¹⁴C, or ³H, either directly or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

It is also within the scope of this invention to determine the ability of a compound to interact with XBP-1, IRE-1 alpha, and/or EDEM without the labeling of any of the interactants. For example, a microphysiometer can be used to detect the interaction of a compound with XBP-1 without the labeling of either the compound or the XBP-1 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and XBP-1.

Exemplary target molecules of XBP-1 include: XBP-1-responsive elements, for example, upstream regulatory regions from genes such as α-1 antitrypsin, α-fetoprotein, HLA DRα, as well as the 21 base pair repeat enhancer of the HTLV-1 LTR. An example of an XBP-1-responsive reporter construct is the HLA DRα-CAT construct described in Ono, S. J. et al. (1991) Proc. Natl. Acad. Sci. USA 88:4309-4312. Other examples can include regulatory regions of the chaperone genes such as members of the family of Glucose Regulated Proteins (GRPs) such as GRP78 (BiP) and GRP94 (endoplasmin), as well as other chaperones such as calreticulin, protein disulfide isomerase, Erdj4, EDEM and ERp72. XBP-1 targets are taught, e.g. in Clauss et al. Nucleic Acids Research 1996. 24:1855 also include CRE and TRE sequences.

In another embodiment, a different (i.e., non-XBP-1) molecule acting in a pathway involving XBP-1 that acts upstream (e.g., IRE-1 alpha) or downstream (e.g., ATF6α or cochaperone proteins that activate ER resident HspTO proteins, such as p58IPK) of XBP-1 can be included in an indicator composition for use in a screening assay. Compounds identified in a screening assay employing such a molecule would also be useful in modulating XBP-1 activity, albeit indirectly. IRE-1 alpha is one exemplary IRE-1 alpha substrate (e.g., the autophosphorylation of IRE-1 alpha). In another embodiment, the endoribonuclease activity of IRE-1 alpha can be measured, e.g., by detecting the splicing of XBP-1 using techniques that are known in the art. The activity of IRE-1 alpha can also be measured by measuring the modulation of biological activity associated with XBP-1.

In another embodiment, a different (i.e., non-XBP-1) molecule acting in a pathway involving XBP-1 that acts upstream (e.g., IRE-1 alpha) or downstream (e.g., ATF6α or cochaperone proteins that activate ER resident HspTO proteins, such as p58^{IPK}) of XBP-1 can be included in an indicator composition for use in a screening assay.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell. In a preferred embodiment, the cell is a human cell. In another preferred embodiment the cell is a hepatocyte.

The cells of the invention can express endogenous XBP-1, IRE-1 alpha, and/or EDEM, or can be engineered to do so. For example, a cell that has been engineered to express the XBP-1 protein and/or a non XBP-1 protein can be produced by introducing into the cell an expression vector encoding the protein.

In one embodiment, to specifically assess the role of agents that modulate the expression and/or activity of unspliced or spliced XBP-1 protein, retroviral gene transduction of cells deficient in XBP-1 with spliced XBP-1 or a form of XBP-1 which cannot be spliced can be performed. For example, a construct in which mutations at in the loop structure of XBP-1 (e.g., positions −1 and +3 in the loop structure of XBP-1) can be generated. Expression of this construct in cells results in production of the unspliced form of XBP-1 only. Using such constructs, the ability of a compound to modulate a particular form of XBP-1 can be detected. In one embodiment, a compound modulates one form of XBP-1, e.g., spliced XBP-1, without modulating the other form, e.g., unspliced XBP-1.

In another embodiment, the invention provides for screening assays to identify compounds which alter the ratio of spliced XBP-1 to unspliced XBP-1 or the ratio of unspliced XBP-1 to spliced XBP-1. Only the spliced form of XBP-1 mRNA activates gene transcription. Unspliced XBP-1 mRNA inhibits the activity of spliced XBP-1 mRNA. As explained above, human and murine XBP-1 mRNA contain an open reading frame (ORF1) encoding bZIP proteins of 261 and 267 amino acids, respectively. Both mRNA's also contain another ORF, ORF2, partially overlapping but not in frame with ORF1. ORF2 encodes 222 amino acids in both human and murine cells. Human and murine ORF1 and ORF2 in the XBP-1 mRNA share 75% and 89% identity respectively. In response to ER stress, XBP-1 mRNA is processed by the ER transmembrane endoribonuclease and kinase IRE-1 which excises an intron from XBP-1 mRNA. In murine and human cells, a 26 nucleotide intron is excised. Splicing out of 26 nucleotides in murine cells results in a frame shift at amino acid 165. This causes removal of the C-terminal 97 amino acids from the first open reading frame (ORF1) and addition of the 212 amino from ORF2 to the N-terminal 164 amino acids of ORF1 containing the b-ZIP domain. In mammalian cells, this splicing event results in the conversion of an approximately 267 amino acid unspliced XBP-1 protein to a 371 amino acid spliced XBP-1 protein. The spliced XBP-1 then translocates into the nucleus where it binds to its target sequences to induce their transcription.

Compounds that alter the ratio of unspliced to spliced XBP-1 or spliced to unspliced XBP-1 can be useful to modulate the biological activities of XBP-1, e.g., in modulation of the UPR, modulation of cellular differentiation, modulation of IL-6 production, modulation of immunoglobulin production, modulation of the proteasome pathway, modulation of protein folding and transport, modulation of terminal B cell differentiation, and modulation of apoptosis. The compounds can also be used to treat disorders that would benefit from modulation of XBP-1 expression and/or activity, e.g., autoimmune disorders, and malignancies.

The techniques for assessing the ratios of unspliced to spliced XBP-1 and spliced to unspliced XBP-1 are routine in the art. For example, the two forms can be distinguished based on their size, e.g., using northern blots or western blots. Because the spliced form of XBP-1 comprises an exon not found in the unspliced form, in another embodiment, antibodies that specifically recognize the spliced or unspliced form of XBP-1 can be developed using techniques well known in the art (Yoshida et al. 2001. Cell. 107:881). In addition, PCR can be used to distinguish spliced from unspliced XBP-1. For example, as described herein, primer sets can be used to amplify XBP-1 where the primers are derived from positions 410 and 580 of murine XBP-1, or corresponding positions in related XBP-1 molecules, in order to amplify the region that encompasses the splice junction. A fragment of 171 base pairs corresponds to unspliced XBP-1 mRNA. An additional band of 145 bp corresponds to the spliced form of XBP-1. The ratio of the different forms of XBP-1 can be determined using these or other art recognized methods.

Recombinant expression vectors that can be used for expression of XBP-1, IRE-1 alpha, and/or EDEM in the indicator cell are known in the art. For example, the XBP-1 cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for XBP-1 or a molecule in a signal transduction pathway involving XBP-1 (e.g., human, murine and yeast) are known in the art and can be used for the design of PCR primers that allow for amplification of a cDNA by standard PCR methods or for the design of a hybridization probe that can be used to screen a cDNA library using standard hybridization methods.

Following isolation or amplification of a cDNA molecule encoding, for example, XBP-1, the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule in a form suitable for expression of the nucleic acid molecule in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufman et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the □-fetoprotein promoter (Campes and Tilghman (1989) Genes Dev. 3:537-546).

Vector DNA can be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on a separate vector from that encoding XBP-1 or, more preferably, on the same vector. Cells stably transfected with the introduced nucleic acid can be identified by drug selection (e.g., cells that have incorporated the selectable marker gene will survive, while the other cells die).

In one embodiment, within the expression vector coding sequences are operatively linked to regulatory sequences that allow for constitutive expression of the molecule in the indicator cell (e.g., viral regulatory sequences, such as a cytomegalovirus promoter/enhancer, can be used). Use of a recombinant expression vector that allows for constitutive expression of, for example, XBP-1, IRE-1 alpha, and/or EDEM in the indicator cell is preferred for identification of compounds that enhance or inhibit the activity of the molecule. In an alternative embodiment, within the expression vector the coding sequences are operatively linked to regulatory sequences of the endogenous gene for XBP-1 (i.e., the promoter regulatory region derived from the endogenous gene). Use of a recombinant expression vector in which expression is controlled by the endogenous regulatory sequences is preferred for identification of compounds that enhance or inhibit the transcriptional expression of the molecule.

C. Cell-Free Assays

In another embodiment, the indicator composition is a cell free composition. XBP-1, IRE-1 alpha, and/or EDEM protein expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate XBP-1, IRE-1 alpha, and/or EDEM activity are identified based on their ability to modulate the interaction of XBP-1, IRE-1 alpha, and/or EDEM with a target molecule to which XBP-1, IRE-1 alpha, and/or EDEM binds. The target molecule can be a DNA molecule, e.g., an XBP-1-responsive element, such as the regulatory region of a chaperone gene) or a protein molecule. Suitable assays are known in the art that allow for the detection of protein-protein interactions (e.g., immunoprecipitations, two-hybrid assays and the like) or that allow for the detection of interactions between a DNA binding protein with a target DNA sequence (e.g., electrophoretic mobility shift assays, DNAse I footprinting assays, chromatin immunoprecipitations assays and the like). By performing such assays in the presence and absence of test compounds, these assays can be used to identify compounds that modulate (e.g., inhibit or enhance) the interaction of XBP-1, IRE-1 alpha, and/or EDEM with a target molecule.

In one embodiment, the amount of binding of XBP-1, IRE-1 alpha, and/or EDEM to the target molecule in the presence of the test compound is greater than the amount of binding of XBP-1, IRE-1 alpha, and/or EDEM to the target molecule in the absence of the test compound, in which case the test compound is identified as a compound that enhances binding of XBP-1, IRE-1 alpha, and/or EDEM to a target. In another embodiment, the amount of binding of the XBP-1, IRE-1 alpha, and/or EDEM to the target molecule in the presence of the test compound is less than the amount of binding of the XBP-1, IRE-1 alpha, and/or EDEM to the target molecule in the absence of the test compound, in which case the test compound is identified as a compound that inhibits binding of XBP-1 to the target.

Binding of the test compound to XBP-1, IRE-1 alpha, and/or EDEM can be determined either directly or indirectly as described above. Determining the ability of XBP-1, IRE-1 alpha, and/or EDEM protein to bind to a test compound can also be accomplished using a technology such as real-time Biomolecular Interaction Analysis (BIA) (Sjolander, S, and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In the methods of the invention for identifying test compounds that modulate an interaction between XBP-1 XBP-1, IRE-1 alpha, and/or EDEM. protein and a target molecule, the complete XBP-1, IRE-1 alpha, and/or EDEM protein can be used in the method, or, alternatively, only portions of the protein can be used. For example, an isolated XBP-1 b-ZIP structure (or a larger subregion of XBP-1 that includes the b-ZIP structure) can be used. In another example, a form of XBP-1 comprising the splice junction can be used (e.g., a portion including from about nucleotide 506 to about nucleotide 532). The degree of interaction between the protein and the target molecule can be determined, for example, by labeling one of the proteins with a detectable substance (e.g., a radiolabel), isolating the non-labeled protein and quantitating the amount of detectable substance that has become associated with the non-labeled protein. The assay can be used to identify test compounds that either stimulate or inhibit the interaction between the XBP-1, IRE-1 alpha, and/or EDEM protein and a target molecule. A test compound that stimulates the interaction between the protein and a target molecule is identified based upon its ability to increase the degree of interaction between, e.g., spliced XBP-1 and a target molecule as compared to the degree of interaction in the absence of the test compound and such a compound would be expected to increase the activity of spliced XBP-1 in the cell. A test compound that inhibits the interaction between the protein and a target molecule is identified based upon its ability to decrease the degree of interaction between the protein and a target molecule as compared to the degree of interaction in the absence of the compound and such a compound would be expected to decrease spliced XBP-1 activity.

In one embodiment of the above assay methods of the present invention, it may be desirable to immobilize either XBP-1, IRE-1 alpha, and/or EDEM or a respective target molecule for example, to facilitate separation of complexed from uncomplexed forms of one or both of the proteins, or to accommodate automation of the assay. Binding of a test compound to, for example, an XBP-1 protein, or interaction of an XBP-1 protein with a target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or XBP-1. protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either an XBP-1 protein or a molecule in a signal transduction pathway involving XBP-1, or a target molecule can be immobilized utilizing conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with protein or target molecules but which do not interfere with binding of the protein to its target molecule can be derivatized to the wells of the plate, and unbound target or XBP-1. protein is trapped in the wells by antibody conjugation. Methods for detecting such complexes, in addition to those described above for the GST-immobilized complexes, include immunodetection of complexes using antibodies reactive with XBP-1 or a molecule in a signal transduction pathway involving XBP-1 or target molecule, as well as enzyme-linked assays which rely on detecting an enzymatic activity associated with the XBP-1, IRE-1 alpha, and/or EDEM, protein or target molecule.

In yet another aspect of the invention, the XBP-1, IRE-1 alpha, and/or EDEM protein or fragments thereof can be used as “bait proteins” e.g., in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with XBP-1 (“binding proteins” or “bp”) and are involved in XBP-1 activity. Such XBP-1-binding proteins are also likely to be involved in the propagation of signals by the XBP-1 proteins or XBP-1 targets such as, for example, downstream elements of an XBP-1-mediated signaling pathway. Alternatively, such XBP-1-binding proteins can be XBP-1 inhibitors.

The two-hybrid system is based on the modular nature of most transcription factors, which consist of separable DNA-binding and activation domains. Briefly, the assay utilizes two different DNA constructs. In one construct, the gene that codes for an XBP-1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an XBP-1 dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the XBP-1 protein or a molecule in a signal transduction pathway involving XBP-1.

C. Assays Using Knock-Down or Knock-Out Cells

In another embodiment, the invention provides methods for identifying compounds that modulate a biological effect of XBP-1, IRE-1 alpha, and/or EDEM using cells deficient in XBP-1 (or e.g., IRE-1 alpha and/or EDEM). For example, cells deficient in which XBP-1, IRE-1 alpha, and/or EDEM is knocked down can be used to identify agents that modulate a biological response regulated by XBP-1, IRE-1 alpha, and/or EDEM by means other than modulating XBP-1, IRE-1 alpha, and/or EDEM itself (i.e., compounds that “rescue” the XBP-1 deficient phenotype). Alternatively, a “conditional knock-out” system, in which the gene is rendered non-functional in a conditional manner, can be used to create deficient cells for use in screening assays. For example, a tetracycline-regulated system for conditional disruption of a gene as described in WO 94/29442 and U.S. Pat. No. 5,650,298 can be used to create cells, or animals from which cells can be isolated, be rendered deficient in XBP-1, IRE-1 alpha, and/or EDEM in a controlled manner through modulation of the tetracycline concentration in contact with the cells.

In the screening method, cells deficient in XBP-1, IRE-1 alpha, and/or EDEM can be contacted with a test compound and a biological response regulated by XBP-1, IRE-1 alpha, and/or EDEM can be monitored. Modulation of the response in cells deficient in XBP-1, IRE-1 alpha, and/or EDEM (as compared to an appropriate control such as, for example, untreated cells or cells treated with a control agent) identifies a test compound as a modulator of the XBP-1, IRE-1 alpha, and/or EDEM regulated response. In another embodiment, to specifically assess the role of agents that modulate unspliced or spliced XBP-1 protein, retroviral gene transduction of cells deficient in XBP-1, to express spliced XBP-1 or a form of XBP-1 which cannot be spliced can be performed. For example, a construct in which mutations at in the loop structure of XBP-1 (e.g., positions −1 and +3 in the loop structure of XBP-1) can be generated. Expression of this construct in cells results in production of the unspliced form of XBP-1 only. Using such constructs, the ability of a compound to modulate a particular form of XBP-1 can be detected. For example, in one embodiment, a compound modulates one form of XBP-1 without modulating the other form.

In one embodiment, the test compound is administered directly to a non-human knock out animal, preferably a mouse (e.g., a mouse in which the XBP-1 gene is conditionally disrupted by means described above, or a mouse in which the motor neuron cells are deficient in XBP-1 as described herein), to identify a test compound that modulates the in vivo responses of cells deficient in XBP-1, IRE-1 alpha, and/or EDEM. In another embodiment, cells deficient in XBP-1, IRE-1 alpha, and/or EDEM are isolated from the non-human XBP-1 or a molecule in a signal transduction pathway involving XBP-1 deficient animal, and contacted with the test compound ex vivo to identify a test compound that modulates a response regulated by XBP-1, IRE-1 alpha, and/or EDEM in the cells

Cells deficient in XBP-1, IRE-1 alpha, and/or EDEM can be obtained from a non-human animals created to be deficient in XBP-1, IRE-1 alpha, and/or EDEM. Preferred non-human animals include monkeys, dogs, cats, mice, rats, cows, horses, goats and sheep. In preferred embodiments, the deficient animal is a mouse. Mice deficient in XBP-1, IRE-1 alpha, and/or EDEM can be made using methods known in the art. Non-human animals deficient in a particular gene product typically are created by homologous recombination. Briefly, a vector is prepared which contains at least a portion of the gene into which a deletion, addition or substitution has been introduced to thereby alter, e.g., functionally disrupt, the endogenous XBP-1 (or e.g., IRE-1 alpha gene). The gene preferably is a mouse gene. For example, a mouse XBP-1 gene can be isolated from a mouse genomic DNA library using the mouse XBP-1 cDNA as a probe. The mouse XBP-1 gene then can be used to construct a homologous recombination vector suitable for modulating an endogenous XBP-1 gene in the mouse genome. In a preferred embodiment, the vector is designed such that, upon homologous recombination, the endogenous gene is functionally disrupted (i.e., no longer encodes a functional protein; also referred to as a “knock out” vector).

Alternatively, the vector can be designed such that, upon homologous recombination, the endogenous gene is mutated or otherwise altered but still encodes functional protein (e.g., the upstream regulatory region can be altered to thereby alter the expression of the endogenous XBP-1 protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced gene has homologously recombined with the endogenous gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, retroviral transduction of donor bone marrow cells from both wild type and null mice can be performed, e.g., with the XBP-1 unspliced, DN or spliced constructs to reconstitute irradiated RAG recipients. This will result in the production of mice whose lymphoid cells express only unspliced, or only spliced XBP-1 protein, or which express a dominant negative version of XBP-1. Cells from these mice can then be tested for compounds that modulate a biological response regulated by XBP-1.

In another embodiment, XBP-1 may be temporally deleted to, for example, circumvent embryonic lethality. For example, as described herein and in C. Hetz et al. (2008) Proc Natl Acad Sci USA In Press, XBP-1 was specifically deleted using XBP-1^{flox} mice bred with Mx1-cre mice (R. Kuhn, et al. (2995) Science 269, 1427) to generate XBP-1^{f/f}; Mx1-cre mice, and subsequently treated with poly(I:C) to induce cre expression and excision of the floxed exon 2 of XBP-1.

In another embodiment, a molecule which mediates RNAi, e.g., double stranded RNA can be used to knock down expression of XBP-1, IRE-1 alpha, and/or EDEM. For example, an XBP-1-specific RNAi vector has been constructed by inserting two complementary oligonucleotides 5′-GGGATTCATGAATGGCCCTTA-3′ into the pBS/U6 vector as described (Sui et al. 2002 Proc Natl Acad Sci USA 99: 5515-5520).

In one embodiment of the screening assay, compounds tested for their ability to modulate a biological response regulated by XBP-1, IRE-1 alpha, and/or EDEM are contacted with deficient cells by administering the test compound to a non-human deficient animal in vivo and evaluating the effect of the test compound on the response in the animal.

The test compound can be administered to a non-knock out animal as a pharmaceutical composition. Such compositions typically comprise the test compound and a pharmaceutically acceptable carrier. As used herein the term “pharmaceutically acceptable carrier” includes any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions. Pharmaceutical compositions are described in more detail below.

In another embodiment, compounds that modulate a biological response regulated by XBP-1, IRE-1 alpha, and/or EDEM are identified by contacting cells deficient in, for example, XBP-1, ex vivo with one or more test compounds, and determining the effect of the test compound on a read-out. In one embodiment, XBP-1, IRE-1 alpha, and/or EDEM deficient cells contacted with a test compound ex vivo can be re-administered to a subject.

For practicing the screening method ex vivo, cells deficient, e.g., in XBP-1, IRE-1 alpha, and/or EDEM, can be isolated from a non-human XBP-1, IRE-1 alpha, and/or EDEM, deficient animal or embryo by standard methods and incubated (i.e., cultured) in vitro with a test compound. Cells (e.g., B cells, hepatocytes, MEFs) can be isolated from e.g., XBP-1, IRE-1 alpha, and/or EDEM deficient animals by standard techniques.

In another embodiment, cells deficient in more than one member of a signal transduction pathway involving XBP-1 can be used in the subject assays.

Following contact of the deficient cells with a test compound (either ex vivo or in vivo), the effect of the test compound on the biological response regulated by XBP-1 or a molecule in a signal transduction pathway involving XBP-1 can be determined by any one of a variety of suitable methods, such as those set forth herein, e.g., including light microscopic analysis of the cells, histochemical analysis of the cells, production of proteins, induction of certain genes, e.g., chaperone genes, lipogenic genes.

D. Test Compounds

A variety of test compounds can be evaluated using the screening assays described herein. The term “test compound” includes any reagent or test agent which is employed in the assays of the invention and assayed for its ability to influence the expression and/or activity of XBP-1, IRE-1 alpha, and/or EDEM. More than one compound, e.g., a plurality of compounds, can be tested at the same time for their ability to modulate the expression and/or activity of, e.g., XBP-1, in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening can be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061-).

The compounds of the present invention can be obtained using any of the numerous approaches in combinatorial library methods known in the art, including:

biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422; Horwell et al. (1996) Immunopharmacology 33:68-; and in Gallop et al. (1994); J. Med. Chem. 37:1233-.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Nall Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310). In still another embodiment, the combinatorial polypeptides are produced from a cDNA library.

Exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms of XBP-1 (or e.g., IRE-1 alpha, and/or EDEM molecules), e.g., dominant negative mutant forms of the molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Nall Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Compounds identified in the subject screening assays can be used in methods of modulating one or more of the biological responses regulated by XBP-1, IRE-1 alpha, and/or EDEM. It will be understood that it may be desirable to formulate such compound(s) as pharmaceutical compositions (described supra) prior to contacting them with cells.

Once a test compound is identified that directly or indirectly modulates, e.g., XBP-1 expression or activity, by one of the variety of methods described hereinbefore, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

E. Computer Assisted Design of Modulators of XBP-1, IRE-1 alpha, and/or EDEM

Computer-based analysis of a protein with a known structure can also be used to identify molecules which will bind to the protein. Such methods rank molecules based on their shape complementary to a receptor site. For example, using a 3-D database, a program such as DOCK can be used to identify molecules which will bind to XBP-1, IRE-1 alpha, and/or EDEM. See DesJarlias et al. (1988) J. Med. Chem. 31:722; Meng et al. (1992) J. Computer Chem. 13:505; Meng et al. (1993) Proteins 17:266; Shoichet et al. (1993) Science 259:1445. In addition, the electronic complementarity of a molecule to a targeted protein can also be analyzed to identify molecules which bind to the target. This can be determined using, for example, a molecular mechanics force field as described in Meng et al. (1992) J. Computer Chem. 13:505 and Meng et al. (1993) Proteins 17:266. Other programs which can be used include CLIX which uses a GRID force field in docking of putative ligands. See Lawrence et al. (1992) Proteins 12:31; Goodford et al. (1985) J. Med. Chem. 28:849; Boobbyer et al. (1989) J. Med. Chem. 32:1083.

Compounds identified in the subject screening assays can be used in methods of modulating one or more of the biological responses regulated by XBP-1, IRE-1 alpha, and/or EDEM. It will be understood that it may be desirable to formulate such compound(s) as pharmaceutical compositions (described supra) prior to contacting them with cells.

Once a test compound is identified that directly or indirectly modulates, e.g., XBP-1, IRE-1 alpha, and/or EDEM expression or activity, by one of the variety of methods described hereinbefore, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

The instant invention also pertains to compounds identified in the subject screening assays.

### III. METHODS OF THE INVENTION

The invention provides methods for modulating, e.g., decreasing, protein aggregation associated with a neurodegenerative disorder and methods for modulating, e.g., decreasing, apoptosis in a motor neuron cell by modulating XBP-1, IRE-1 alpha, and/or EDEM expression and/or activity, in vitro and in vivo. In particular, the ability of a compound to modulate XBP-1, IRE-1 alpha, and/or EDEM can be detected by measuring the ability of the compound to modulate a biological activity of XBP-1, IRE-1 alpha, and/or EDEM, e.g., by measuring modulation of a neurodegenerative diease associated with protein aggregation, modulation of autophagy, modulation of motor neuron survival, modulation of the generation of protein aggregation associated with a neurodegenerative disease, modulation of apoptosis, modulation of the UPR, modulation of the proteasome pathway, modulation of ERAD, and modulation of protein folding and transport.

Such modulatory methods generally involve contacting a cell (e.g., a motor neuron cell) with an agent that modulates, e.g., decreases, the expression and/or biological activity of XBP-1, IRE-1 alpha, and/or EDEM. An agent that modulates, for example, XBP-1 activity can be an agent as described herein, such as a peptide that binds to XBP-1, a peptide that binds to IRE-1 alpha, a peptide that binds to EDEM, an XBP-1 antagonist, an IRE-1 alpha antagonist, an EDEM antagonist, a peptidomimetic of an XBP-1 antagonist, a peptidomimetic of an IRE-1 alpha antagonist, a peptidomimetic of an EDEM1 antagonist, or other small molecule identified using the screening methods described herein. Additional agents include, but are not limited to a nucleic acid molecule that is antisense to a XBP-1 molecule, a nucleic acid molecule that is antisense to a IRE-1 alpha molecule, a nucleic acid molecule that is antisense to a EDEM molecule, an XBP-1 siRNA molecule, an IRE-1 alpha siRNA molecule, an EDEM siRNA molecule, a chemical chaperone, and an activator of autophagy. The claimed methods are not meant to include naturally occurring events. For example, the term “agent” or “modulator” is not meant to embrace endogenous mediators produced by the cells of a subject.

These modulatory methods can be performed in vitro (e.g., by contacting the cell with the agent) or, alternatively, in vivo (e.g., by administering the agent to a subject). As such, the present invention pertains to methods for modulating XBP-1, IRE-1 alpha, and/or EDEM activity for therapeutic purposes. For example, as shown herein, decreasing XBP-1 activity decreases protein aggregation associated with a neurodegenerative disease and, therefore agents that decrease the activity of XBP-1 are useful for the treatment and/or amelioration of at least one symptom, and/or normalization of at least one indicator of the neurodegenerative disease. Similarly, decreasing XBP-1 activity decreases apoptosis of motor neuron cells and, therefore agents that decrease the activity of XBP-1 are useful for the treatment, and/or amelioration of at least one symptom, and/or normalization of at least one indicator of the neurodegenerative disease.

In one embodiment, the method involves administering an agent (e.g., an agent identified by a screening assay described herein), or combination of agents that modulates XBP-1, IRE-1 alpha, and/or EDEM expression or biological activity, as described herein.

The present invention also provides prophylactic methods for preventing and/or delaying the onset of a neurodegenerative disease associated with protein aggregation in a subject at risk of developing such a disease.

Subjects at risk for such disease can be identified by, for example, any or a combination of diagnostic or prognostic assays known in the art. For example, neurodegenerative diseases associated with protein aggregation may be inherited and therefore a subject at risk may be identified based on family history. In addition, genetic testing is available for, e.g., HD, fALS, Alzheimer's. Administration of a prophylactic agent can occur prior to the manifestation of symptoms, such that a disease is prevented or, alternatively, delayed in its progression.

The subject methods employ agents that modulate XBP-1 expression or activity (or the expression or activity of IRE-1 alpha, and/or EDEM) such that an XBP-1 biological activity, e.g., aberrant protein aggeggation associated with a neurodegenerative disease, apoptosis of motor neuron cells, is modulated.

In one embodiment, the methods and compositions of the invention can be used to modulate XBP-1 expression and/or activity in a cell. In one embodiment, the methods and compositions of the invention can be used to modulate IRE-1 alpha expression and/or activity in a cell. In one embodiment, the methods and compositions of the invention can be used to modulate EDEM expression and/or activity in a cell. In one embodiment, the cell is a mammalian cell. In another embodiment, the cell is a human cell. In one embodiment, the cell is a motor neuron. Such modulation can occur in vitro or in vivo.

In one embodiment, cells in which, e.g., XBP-1, is modulated in vitro can be introduced, e.g., into an allogeneic subject, or e.g., reintroduced into a subject. In one embodiment, the invention also allows for modulation of XBP-1 in vivo, by administering to the subject an amount of a modulator of XBP-1 such that at least one symptom or indicator of a neurodegenerative disease associated with protein aggregation in a subject is modulated.

In one embodiment, a modulatory agent of the invention directly affects the expression and/or activity of XBP-1, IRE-1 alpha, and/or EDEM. In another embodiment, the expression of XBP-1, IRE-1 alpha, and/or EDEM is modulated. In another embodiment, the post-translational modification of XBP-1, IRE-1 alpha, and/or EDEM is modulated. In another embodiment, the activity of XBP-1, IRE-1 alpha, and/or EDEM, is modulated, e.g., aberrant protein aggeggation associated with a neurodegenerative disease, apoptosis of motor neuron cells. In one embodiment, the agent modulates the interaction of XBP-1, IRE-1 alpha, and/or EDEM with a DNA molecule to which XBP-1, IRE-1 alpha, and/or EDEM, binds. In another embodiment, a modulatory agent of the invention indirectly affects the expression, post-translational modification, and/or activity of XBP-1, IRE-1 alpha, and/or EDEM.

The term “subject” is intended to include living organisms but preferred subjects are mammals. Examples of subjects include mammals such as, e.g., humans, monkeys, dogs, cats, mice, rats cows, horses, goats, and sheep.

Identification of compounds that modulate the biological effects of XBP-1, IRE-1 alpha, and/or EDEM by directly or indirectly modulating XBP-1, IRE-1 alpha, and/or EDEM activity allows for selective manipulation of these biological effects in a variety of clinical situations using the modulatory methods of the invention. For example, the inhibitory methods of the invention (i.e., methods that use an inhibitory agent) can result in decreased expression, processing, post-translational modification, and/or activity of spliced XBP-1, which decreases, e.g., aberrant protein aggeggation associated with a neurodegenerative disease, apoptosis of motor neuron cells.

In one embodiment, the stimulatory methods of the invention can be used to increase the expression, processing, post-translational modification, and/or activity of a negative regulator of XBP-1 (e.g., unspliced XBP-1 or a dominant negative form of XBP-1) to inhibit e.g., aberrant protein aggeggation associated with a neurodegenerative disease, apoptosis of motor neuron cells. For example, the XBP-1 unspliced protein is an example of an ubiquitinated and hence extremely unstable protein. XBP-1 spliced protein is not ubiquitinated, and has a much longer half life than unspliced XBP-1 protein. Proteasome inhibitors, for example, block ubiquitination, and hence stabilize XBP-1 unspliced but not spliced protein. Thus, the ratio of unspliced to spliced XBP-1 protein increases upon treatment with proteasome inhibitors. Since unspliced XBP-1 protein actually inhibits the function of the spliced protein, treatment with proteasome inhibitors blocks the activity of spliced XBP-1.

Modulation of XBP-1 activity, therefore, provides a means to regulate disorders arising from aberrant XBP-1 activity in various disease states. Thus, to treat and/or prevent a disorder wherein inhibition of a biological effect of spliced XBP-1 is desirable, such as a disorder that would benefit from reduced aberrant protein aggeggation associated with a neurodegenerative disease, apoptosis of motor neuron cells is beneficial, an inhibitory method of the invention is selected such that spliced XBP-1 activity and/or expression is inhibited or a stimulatory method is selected which selectively stimulates the expression and/or activity of a negative regulator of XBP-1.

Examples of disorders in which such inhibitory methods can be useful include, aberrant protein aggeggation associated with a neurodegenerative disease, apoptosis of motor neuron cells, Amyotrophic Lateral Sclerosis (ALS), Huntington's Disease (HD), Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCAT), spinocerebellar ataxia type 17 (SCA17), chronic liver diseases, familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, or subacute sclerosing panencephalitis.

Application of the modulatory methods of the invention for the prevention, treatment, and/or amelioration of at least one symptom, or normalization of at least one indicator of a disorder can result in curing the disorder, a decrease in at least one symptom associated with the disorder, either in the long term or short term (i.e., amelioration of the condition) or simply a transient beneficial effect to the subject.

The modulatory methods of the invention can be practiced either in vitro or in vivo. For practicing the method in vitro, cells can be obtained from a subject by standard methods and incubated (i.e., cultured) in vitro with a stimulatory or inhibitory compound of the invention to stimulate or inhibit, respectively, the activity of XBP-1, IRE-1 alpha, and/or EDEM. Methods for isolating cells are known in the art.

Cells treated in vitro with an inhibitory compound can be administered to a subject to influence the biological effects of XBP-1, IRE-1 alpha, and/or EDEM. For example, cells can be isolated from a subject, expanded in number in vitro and the activity of, e.g., spliced XBP-1, activity in the cells using a stimulatory agent, and then the cells can be readministered to the same subject, or another subject tissue compatible with the donor of the cells. Accordingly, in another embodiment, the modulatory method of the invention comprises culturing cells in vitro with e.g., an XBP-1 modulator and further comprises administering the cells to a subject. For administration of cells to a subject, it may be preferable to first remove residual compounds in the culture from the cells before administering them to the subject. This can be done for example by gradient centrifugation of the cells or by washing of the tissue. For further discussion of ex vivo genetic modification of cells followed by readministration to a subject, see also U.S. Pat. No. 5,399,346 by W. F. Anderson et al.

In other embodiments, an inhibitory compound is administered to a subject in vivo. Such methods can be used to treat neurological disorders associated with aberrant protein aggregation, e.g., as detailed above.

For agents that comprise nucleic acids (e.g., recombinant expression vectors encoding, e.g., XBP-1, IRE-1 alpha, and/or EDEM; antisense RNA; or e.g., XBP-1, IRE-1 alpha, and/or EDEM derived peptides), the compounds can be introduced into cells of a subject using methods known in the art for introducing nucleic acid (e.g., DNA) into cells. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ΨCrip, ΨCre, Ψ2 and ΨAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

In one embodiment, the inhibitory compounds can be administered to a subject as a pharmaceutical composition. In one embodiment, the invention is directed to an active compound (e.g., a modulator of XBP-1) and a carrier. Such compositions typically comprise the inhibitory compounds, e.g., as described herein or as identified in a screening assay, e.g., as described herein, and a pharmaceutically acceptable carrier. Pharmaceutically acceptable carriers and methods of administration to a subject are described herein.

In one embodiment, the active compounds of the invention are administered in combination with other agents. For example, in one embodiment, an active compound of the invention, e.g., a compound that modulates an XBP-1, IRE-1 alpha, and/or EDEM signal transduction pathway (e.g., by directly modulating XBP-1 activity) is administered with another compound known in the art to be useful in treatment of a particular condition or disease. Non-limiting examples of art recognized agents useful in the treatment of a neurodegenerative disease associated with protein aggregation are, Riluzole, Baclofen, tizanadine, nonsteroidal anti-inflammatory drugs (NSAIDs), tramadol, antipsychotics (haloperidol, chlorpromazine, olanzapine), antidepressants (fluoxetine, sertraline hydrochloride, nortriptyline), tranquilizers (benzodiazepines, paroxetine, venlafaxin, beta-blockers), mood-stabilizers (lithium, valproate, carbamazepine), botulinum toxin, Cholinesterase inhibitors (Donepezil, Rivastigmine, Galantamine), Memantine dopamine agonists, levodopa, Symmetrel, Anticholinergics Eldepryl, Deprenyl, Tasmar, Comtan (COMT Inhibitors), etc

Compounds that can be used in the methods of the invention are described in further detail below.

A. Inhibitory Compounds

The methods of the invention using inhibitory compounds which inhibit the expression and/or activity of spliced XBP-1, IRE-1 alpha, and/or EDEM can be used in the prevention and/or treatment of disorders in which spliced XBP-1 activity is undesirably enhanced, stimulated, upregulated or the like, For example, a neurodegenerative disorder associate with aberrant protein aggregation, e.g., Amyotrophic Lateral Sclerosis (ALS), Huntington's Disease (HD), Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCAT), spinocerebellar ataxia type 17 (SCA17), chronic liver diseases, familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, or subacute sclerosing panencephalitis.

In one embodiment of the invention, an inhibitory compound can be used to inhibit (e.g., specifically inhibit) the expression or activity of spliced XBP-1.

Inhibitory compounds of the invention can be, for example, intracellular binding molecules that act to specifically inhibit the expression or activity e.g., of XBP-1, IRE-1 alpha, and/or EDEM. As used herein, the term “intracellular binding molecule” is intended to include molecules that act intracellularly to inhibit the processing expression or activity of a protein by binding to the protein or to a nucleic acid (e.g., an mRNA molecule) that encodes the protein. Examples of intracellular binding molecules, described in further detail below, include antisense nucleic acids, peptidic compounds that inhibit the interaction of XBP-1, IRE-1 alpha, and/or EDEM with a target molecule and chemical agents that specifically inhibit XBP-1, IRE-1 alpha, and/or EDEM activity.

i. Antisense or siRNA Nucleic Acid Molecules

In one embodiment, an inhibitory compound of the invention is an antisense nucleic acid molecule that is complementary to a gene encoding XBP-1, IRE-1 alpha, and/or EDEM, or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the known nucleotide sequence for the coding strand of the XBP-1, IRE-1 alpha, and/or EDEM gene and thus the known sequence of the XBP-1, IRE-1 alpha, and/or EDEM mRNA, antisense nucleic acids of the invention can be designed according to the rules of Watson and Crick base pairing. The antisense nucleic acid molecule can be complementary to the entire coding region of an mRNA, but more preferably is antisense to only a portion of the coding or noncoding region of an mRNA. For example, the antisense oligonucleotide can be complementary to the region surrounding the translation start site of an XBP-1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. To inhibit expression in cells, one or more antisense oligonucleotides can be used.

Alternatively, an antisense nucleic acid can be produced biologically using an expression vector into which all or a portion of a cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector can be introduced into cells using a standard transfection technique.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid molecule of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation mRNAs. A ribozyme having specificity e.g., for an XBP-1, IRE-1 alpha, or ATF6α-encoding nucleic acid can be designed based upon the nucleotide sequence of the cDNA. For example, a derivative of a Tetrahymena L-19 IVS RNA can be constructed in which the nucleotide sequence of the active site is complementary to the nucleotide sequence to be cleaved in, e.g., an XBP-1-encoding mRNA. See, e.g., Cech et al. U.S. Pat. No. 4,987,071 and Cech et al. U.S. Pat. No. 5,116,742. Alternatively, XBP-1 mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of a gene (e.g., an XBP-1 promoter and/or enhancer) to form triple helical structures that prevent transcription of a gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In another embodiment, a compound that promotes RNAi can be used to inhibit expression of XBP-1, IRE-1 alpha, and/or EDEM. RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell TR, and Doering TL. 2003. Trends Microbiol. 11:37-43; Bushman F. 2003. Mol. Therapy. 7:9-10; McManus MT and Sharp PA. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabsor Ambion. In one embodiment one or more of the chemistries described above or known in the art for use in antisense RNA can be employed in molecules that mediate RNAi. A working example of XBP-1 specific RNAi in which an XBP-1-specific RNAi vector was constructed by inserting two complementary oligonucleotides for 5′-GGGATTCATGAATGGCCCTTA-3′ (SEQ ID NO: 9) into the pBS/U6 vector.

Exemplary siRNA molecules specific for the unspliced form of murine XBP-1 are shown below:

Beginning at position 711:

(SEQ ID NO.: 32)
Sense strand siRNA: GUUGGACCCUGUCAUGUUUtt
(SEQ ID NO.: 33)
Antisense strand siRNA: AAACAUGACAGGGUCCAACtt

Beginning at position 853:

(SEQ ID NO.: 34)
Sense strand siRNA: GCCAUUAAUGAACUCAUUCtt
(SEQ ID NO.: 35)
Antisense strand siRNA: GAAUGAGUUCAUUAAUGGCtt

Exemplary siRNA molecules specific for the spliced form of murine XBP-1 are shown below:

Beginning at position 746:

(SEQ ID NO.: 36)
Sense strand siRNA: GAAGAGAACCACAAACUCCUU
(SEQ ID NO.: 37)
Antisense strand siRNA: GGAGUUUGUGGUUCUCUUCUU

Beginning at position 1307:

(SEQ ID NO.: 38)
Sense strand siRNA: GAGGAUCACCCUGAAUUCAUU
(SEQ ID NO.: 39)
Antisense strand siRNA: UGAAUUCAGGGUGAUCCUCUU

Exemplary siRNA molecules specific for the unspliced form of human XBP-1 are shown below:

Beginning at position 729:

(SEQ ID NO.: 44)
Sense strand siRNA: CUUGGACCCAGUCAUGUUCUU
(SEQ ID NO.: 45)
Antisense strand siRNA: GAACAUGACUGGGUCCAAGUU

Beginning at position 1079:

(SEQ ID NO.: 46)
Sense strand siRNA: AUCUGCUUUCAUCCAGCCAUU
(SEQ ID NO.: 47)
Antisense strand siRNA: UGGCUGGAUGAAAGCAGAUUU

Exemplary siRNA molecules specific for the spliced form of human XBP-1 are shown below:

Beginning at position 884:

(SEQ ID NO.: 48)
Sense strand siRNA: GCCCCUAGUCUUAGAGAUAUU
(SEQ ID NO.: 49)
Antisense strand siRNA: UAUCUCUAAGACUAGGGGCUU

Beginning at position 1108:

(SEQ ID NO.: 50)
Sense strand siRNA: GAACCUGUAGAAGAUGACCUU
(SEQ ID NO.: 51)
Antisense strand siRNA: GGUCAUCUUCUACAGGUUCUU

Exemplary siRNA molecules specific for human IRE-1 alpha are shown below:

Beginning at position 443

Sense strand siRNA: GUUCAGAUGGAAUCCUCUAUU
Antisense strand siRNA: UAGAGGAUUCCAUCUGAACUU

Beginning at position 1865

Sense strand siRNA: UUGUGUACCGGGGCAUGUUUU
Antisense strand siRNA: AACAUGCCCCGGUACACAAUU

Exemplary siRNA molecules specific for murine IRE-1 alpha are shown below:

Beginning at position 902

Sense strand siRNA: GUGGGGCGCAUCACCAAGUUU
Antisense strand siRNA: ACUUGGUGAUGCGCCCCACUU

Beginning at position 2197

Sense strand siRNA: CAUUCUCCUCUCCAUGCCCUU
Antisense strand siRNA: GGGCAUGGAGAGGAGAAUGUU

Exemplary siRNA molecules specific for human EDEM are shown below:

Beginning at position 1289

Sense strand siRNA: UGAAGGAGAAGGAGACCCUUU
Antisense strand siRNA: AGGGUCUCCUUCUCCUUCAUU

Beginning at position 3355

Sense strand siRNA: CAUCUCUCUUUGAACUCACUU
Antisense strand siRNA: GUGAGUUCAAAGAGAGAUGUU

Exemplary siRNA molecules specific for murine EDEM are shown below:

Beginning at position 1307

Sense strand siRNA: GCCUGCAAUGAAGGAGAAGUU
Antisense strand siRNA: CUUCUCCUUCAUUGCAGGCUU

Beginning at position 3063

Sense strand siRNA: CCACUGCUACAGACAGCUCUU
Antisense strand siRNA: GAGCUGUCUGUAGCAGUGGUU

ii. Peptidic Compounds

In another embodiment, an inhibitory compound of the invention is a peptidic compound derived from the XBP-1, IRE-1 alpha, and/or EDEM amino acid sequence. For example, in one embodiment, the inhibitory compound comprises a portion of, e.g., XBP-1 (or a mimetic thereof) that mediates interaction of XBP-1 with a target molecule such that contact of XBP-1 with this peptidic compound competitively inhibits the interaction of XBP-1 with the target molecule.

The peptidic compounds of the invention can be made intracellularly in cells by introducing into the cells an expression vector encoding the peptide. Such expression vectors can be made by standard techniques using oligonucleotides that encode the amino acid sequence of the peptidic compound. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like).

In addition, dominant negative proteins (e.g., of XBP-1) can be made which include XBP-1 molecules (e.g., portions or variants thereof) that compete with native (i.e., wild-type) molecules, but which do not have the same biological activity. Such molecules effectively decrease, e.g., XBP-1 activity in a cell. For example, the peptide compound can be lacking part of an XBP-1 transcriptional activation domain, e.g., can consist of the portion of the N-terminal 136 or 188 amino acids of the spliced form of XBP-1.

iii. Other Agents

In one embodiment, the expression of spliced XBP-1 can be inhibited using an agent that inhibits a signal that increases XBP-1 expression, processing, post-translational modification or activity in a cell. Both IL-4 and IL-6 have been shown to increase transcription of XBP-1 (Wen et al. 1999. Int. Journal of Oncology 15:173) and Iwakoshi, et al. (2003) Nat. Immunol. 4 (4): 321-9). Accordingly, in one embodiment, an agent that inhibits a signal transduced by IL-4 or IL-6 can be used to downmodulate XBP-1 expression and, thereby, decrease the activity of spliced XBP-1 in a cell. For example, in one embodiment, an agent that inhibits a STAT-6 dependent signal can be used to decrease the expression of XBP-1 in a cell.

Other inhibitory agents that can be used to specifically inhibit the activity of XBP-1, IRE-1 alpha, and/or EDEM are chemical compounds that directly inhibit expression, processing, post-translational modification, and/or activity of, e.g., an XBP-1 target protein activity or inhibit the interaction between, e.g., XBP-1 and target molecules. Such compounds can be identified using screening assays that select for such compounds, as described in detail above as well as using other art recognized techniques.

In one embodiment, an inhibitory compound is a chemical chaperone. As used herein, a “chemical chaperone” is a compound known to stabilize protein conformation against denaturation (e.g., chemical denaturation, thermal denaturation), thereby preserving protein structure and function (Welch et al. Cell Stress Chaperones 1:109-115, 1996; incorporated herein by reference). Chemical chaperones have been shown in certain instances to correct folding/trafficking defects seen in such diseases as cystic fibrosis (Fischer et al. Am. J. Physiol. Lung Cell Mol. Physiol. 281:L52-L57, 2001; incorporated herein by reference), prion-associated diseases, nephrogenic diabetes insipidus, and cancer (Bai et al. Journal of Pharmacological and Toxicological Methods 40(1):39-45, July 1998; incorporated herein by reference).

In one embodiment, a “chemical chaperone” is a small molecule or low molecular weight compound. Preferably, the “chemical chaperone” is not a protein. Examples of “chemical chaperones include glycerol, deuterated water (D20), dimethylsulfoxide (DMSO), trimethylamine N-oxide (TMAO), glycine betaine (betaine), glycerolphosphocholine (GPC) (Burg et al. Am. J. Physiol. (Renal Physiol. 43):F762-F765, 1998; incorporated herein by reference), 4-phenyl butyrate or 4-phenyl butyric acid (PBA), methylamines, and tauroursodeoxycholic acid (TUDCA), taurin, methylamine and deoxyspergualin (see Brown et al., Cell Stress Chaperones 1:117-125, 1996; Jiang et al., Amer J. Physiol.-Cell Physiol. 44:C171-C178, 1998). (Rubenstein et al., J. Clin. Invest. 100:2457-2465, 1997), sodium butyrate (Cheng et al., Am. J. Physiol. 268:L615-624, 1995) and S-Nitrosoglutathione (Zaman, et al., Biochem Biophys Res Commun 284: 65-70, 2001; Snyder, et al., American Journal of Respiratory and Critical Care Medicine 165: 922-6, 2002; Andersson, et al. Biochemical and Biophysical Research Communication 297(3): 552-557, 2002.).

In another embodiment of the invention, an inhibitory compound is an autophagy activator. As used herein, an “autophagy activator” is any compound that increases autphagy within a cell. An increase in autophagy may be determined as known in the art and described herein. Exemplary, non-limiting autophagy activators are known in the art and include, for example, proteasome inhibitor, tamoxifen, IFN-gamma, trehalose, vinblastine, rapamycin, or its analogues, that inhibit the mammalian target of rapamycin (mTOR) (a negative regulator of autophagy), ganima-benzene hexachloride, or of a derivative thereof which is obtainable by chemical substitution, but has retained said capacity of acting as an inducer or stimulator of autophagy maturation.

An mTor inhibitor may include a rapamycin macrolide such as rapamycin or a salt, analogue or derivatives of rapamycin. Suitable rapamycin macrolides are described in more detail below. An IMPase inhibitor may include a compound described above. mTOR inhibitors include rapamycin and other rapamycin macrolides. A macrolide is a macrocyclic lactone, for example a compound having a 12-membered or larger lactone ring. Lactam macrolides are macrocyclic compounds which have a lactam (amide) bond in the macrocycle in addition to a lactone (ester) bond.

Rapamycin is a lactam macrolide produced by Streptomyces hygroscopicus (McAlpine J. B. et al. J. Antibiotics (1991) 44: 688; Schreiber, S. L. et al. J. Am. Chem. Soc. (1991) 113:7433; U.S. Pat. No. 3,929,992). A rapamycin macrolide as described herein may include rapamycin or a salt, analogue or derivative of rapamycin.

Suitable rapamycin analogues well known in the art (see for example WO 94/09010 and Wa 96/41807) and include 40-0-(2-hydroxy)ethyl-rapamycin, 32-deoxo-rapamycin, 16-O-pent-2-ynyl-32-deoxo-rapamycin, 16-a-pent-2-ynyl-32-deoxO-40-O-(2-hydroxyethyl)-rapamycin, 16-O-pent-2-ynyl-32-CS)-dihydro-rapamycin and 16-a-pent-2-ynyl-32-(S)-dihydro-40-0-C2 hydroxyethyl)-rapamycin. Other rapamycin analogues include carboxylic acid esters as set out in WO 92/05179, amide esters as set out in U.S. Pat. No. 5,118,677, carbamates as set out in U.S. Pat. No. 5,118,678, fluorinated esters as set out in USS, 100,883, acetals as set out in U.S. Pat. No. 5,151,413, silyl ethers as set out in U.S. Pat. No. 5,120,842 and arylsulfonates and sulfamates as set out in U.S. Pat. No. 5,177,203. Other rapamycin analogues which may be used in accordance with the invention may have the methoxy group at the position 16 replaced with alkynyloxy as set out in WO 95/16691. Rapamycin analogues are also disclosed in WO 93/11130, WO 94/02136, WO 94/02385 and Administration of a compound for the treatment of a disorder, as described herein, is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

### IV. DIAGNOSTIC METHODS

As described in the appended Examples, it has been discovered that subjects, e.g., subjects diagnosed with sALS and fALS, exhibit increased expression of genes whose expression is regulated by XBP-1, e.g., “XBP-1 regulated genes”, e.g., ERdj4, EDEM, WFS1, Grp58, PDI, and XBP-1. Accordingly, the present invention also provides methods for determining whether a subject is at risk of developing of has developed a neurodegenerative disease, e.g., ALS associated with aberrant protein aggregation. Such a subject may be one in which the amount of a “marker”, e.g., XBP-1, ERdj4, EDEM, WFS1, Grp58, PDI, e.g., the mRNA and/or protein level and/or activity of the marker, is greater than the level of the marker as compared to a normal or control subject, and who is not at risk of or has not developed a neurodegenerative disease associated with aberrant protein aggregation. In one embodiment, such a subject is identified by determining the level of marker mRNA in a biological sample from the subject. In another embodiment, such a subject is identified by determining the level of marker protein in a biological sample from the subject. In yet another embodiment, such a subject is identified by determining the level of marker activity in a biological sample from the subject.

As used herein, the term “amount”, with respect to marker present in a cell or sample refers to either (a) an absolute amount as measured in molecules, moles or weight per unit volume or cell or (b) a relative amount as designated, for example, by a numerical rating from 0 to 5.

The level or amount of marker in a cell or a sample derived from a subject is “altered” (“increased or decreased” or “higher or lower” than the normal level or amount of marker, if the amount of marker is greater or less, respectively, than a control amount, i.e., a negative control amount, by an amount that is greater than the standard error of the assay employed to assess the amount. The level or amount of marker in a cell or a sample derived from a subject can be considered “higher” or “lower” than the control amount if the difference in the control amount and the sample amount is at least about two, and preferably at least about three, four, or five times, higher or lower, respectively, than the standard error of control and sample measurements of marker.

The term “negative control level” or “negative control amount” of a marker, refers to the level of the marker in a cell or a sample derived from a subject not afflicted with or not at risk of developing a neurodegenerative disease associated with aberrant protein aggregation.

The term “positive control level” or “positive control amount” of a marker, refers to the level of the marker in a cell or a sample derived from a subject known to be afflicted with a neurodegenerative disease associated with aberrant protein aggregation.

The “positive control level” and/or the “negative control level” may, for example, be determined by calculating the average level of marker present in cells or tissues that are known to express the marker.

In general, it is preferable that the difference between the level of a marker in a sample from a subject being treated for a neurodegenerative disease associated with aberrant protein aggregation and the level of the marker in control sample, is as great as possible. Although this difference can be as small as the limit of detection of the method for determining the level it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the standard error of the assessment method.

An alteration in the level of a marker in control (e.g., a negative control, e.g., a tissue from a subject that does not have or is not at risk of developing a neurodegenerative disease associated with aberrant protein aggregation) can be assessed in a variety of ways. In one embodiment, the amount is assessed by assessing the level of marker in cells in which aberrant protein aggregates do not form, e.g., non-neural tissue, and by comparing the foregoing normal level of marker with the amount of marker in the cells which are suspected of being afflicted, e.g., neural tissue.

Alternatively, and particularly as further information becomes available as a result of routine performance of the methods described herein, population-average values for “normal” level of a marker may be used. In other embodiments, the “normal” level of a marker may be determined by assessing the level of the marker in a subject sample obtained from a non-afflicted subject, from a subject sample obtained from a subject before the suspected onset of, e.g., a neurodegenerative disease associated with aberrant protein aggregation, in the subject, from archived subject samples, and the like.

A “higher level of expression and/or activity” of the marker refers to an expression level and/or activity in a test sample that is greater than the standard error of the assay employed to assess expression and/or activity, and is preferably at least twice, and more preferably three, four, five or ten or more times the expression level and/or activity of the marker in a negative control sample (e.g., a sample from a healthy subject not afflicted with or not at risk of a neurodegenerative disease associated with aberrant protein aggregation) and preferably, the average expression level and/or activity of the marker in several control samples.

A “lower level of expression and/or activity” of a marker refers to an expression level and/or activity in a test sample that is greater than the standard error of the assay employed to assess expression and/or activity, but is preferably at least twice, and more preferably three, four, five or ten or more times less than the expression level of the marker in a negative control sample and preferably, the average expression level and/or activity of the marker in several control samples.

As used herein, “known positive standard” or “positive control” refers to one or more of a level of a marker. A known positive standard preferably reflects such levels characteristic of a neurodegenerative disease associated with aberrant protein aggregation.

As used herein, “known negative standard” or “negative control” refers to one or more of a level of a marker. A known negative standard preferably reflects such levels characteristic of the absence of a neurodegenerative disease associated with aberrant protein aggregation

Reagents for generating a known positive and negative standards include, without limitation, cells from a subject who does not have or is not at risk of a neurodegenerative disease associated with aberrant protein aggregation, cells from a tissue that does not develop aberrant protein aggregates associated with a neurodegenerative disease, and/or cells from a subject that has developed a neurodegenerative disease associated with aberrant protein aggregation. Known standards may also include tissue culture cell lines (including, but not limited to, cell lines that have been manipulated to express a marker or manipulated to lose XBP-1 marker expression).

The methods of the present invention can be practiced in conjunction with any other method used by the skilled practitioner to prognose and/or diagnose a neurodegenerative disease associated with aberrant protein aggregation. For example, the methods of the invention may be performed in conjunction with a biochemical, morphological or cytological analysis of the sample obtained from the subject.

### A. Detecting and Determining the Level of a Marker

Material suitable for use in assays to identify a subject afflicted with or at risk of developing a neurodegenerative disease associated with aberrant protein aggregation can be derived from a variety of sources. For example, nucleic acid molecules (e.g., mRNA or DNA, genomic DNA) or polypeptides can be isolated from a cell from a living or deceased individual using standard methods. Cells can be obtained from biological samples, e.g., from tissue samples or from bodily fluid samples that contain cells, such as blood, urine, semen, or saliva. The term “biological sample” is intended to include tissues, cells and biological fluids containing cells which are isolated from a subject, as well as tissues, cells and fluids present within a subject. Samples useful in the methods of the invention include any tissue, cell, biopsy, or bodily fluid sample that expresses marker. In one embodiment, a sample may be a tissue, a cell, whole blood, serum, plasma, buccal scrape, saliva, cerebrospinal fluid, urine, stool, or bronchoalveolar lavage. In certain embodiments, the tissue sample is a large intestine tissue sample.

Body samples may be obtained from a subject by a variety of techniques known in the art including, for example, by the use of a biopsy or by scraping or swabbing an area or by using a needle to aspirate bodily fluids. Methods for collecting various body samples are well known in the art. In particular embodiments, the body sample comprises gastrointestinal tissue samples.

Tissue samples suitable for detecting and determining the level of marker may be fresh, frozen, or fixed according to methods known to one of skill in the art. Suitable tissue samples are preferably sectioned and placed on a microscope slide for further analyses. Alternatively, solid samples, i.e., tissue samples, may be solubilized and/or homogenized and subsequently analyzed as soluble extracts.

Once the sample is obtained any method known in the art to be suitable for detecting and determining the level of marker may be used (either at the nucleic acid or at the protein level). Such methods are well known in the art and include but are not limited to western blots, northern blots, southern blots, immunohistochemistry, ELISA, e.g., amplified ELISA, immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, and nucleic acid amplification methods.

In one embodiment, an antibody-based method is used for detecting and determining the level of marker may be used (either at the nucleic acid or at the protein level). Such methods are the level of marker proteins. Techniques for detecting antibody binding are well known in the art. Antibody binding to marker maybe detected through the use of chemical reagents that generate a detectable signal that corresponds to the level of antibody binding and, accordingly, to the level of marker protein expression. In one of the immunohistochemistry or immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include, but are not limited to, horseradish peroxidase (HRP) and alkaline phosphatase (AP).

In one embodiment of the invention, proteomic methods, e.g., mass spectrometry, are used for detecting and determining the level of marker may be used (either at the nucleic acid or at the protein level). Such methods are the level of marker proteins. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev MoI Diagn 2:549; Li, J., et al. (2002) Clin Chem 48:1296; Laronga, C, et al. (2003) Dis Markers 19:229; Petricoin, E. F., et al (2002) 359:572; Adam, B. L., et al. (2002) Cancer Res 62:3609; Tolson, J., et al. (2004) Lab Invest 84:845; Xiao, Z., et al. (2001) Cancer Res 6 1:6029) can be used to detect and quantitate the marker proteins. Mass spectrometric methods are described in, for example, U.S. Pat. Nos. 5,622,824, 5,605,798 and 5,547,835, the entire contents of each of which are incorporated herein by reference.

In other embodiments, the level of marker is determined by determining the level of expression of marker at the nucleic acid level. Nucleic acid-based techniques for assessing expression are well known in the art and include, for example, determining the level of marker mRNA in a body sample. Many expression detection methods use isolated RNA. Any RNA isolation technique that does not select against the isolation of mRNA can be utilized for the purification of RNA from cells that express marker (see, e.g., Ausubel et al., ed., (1987-1999) Current Protocols in Molecular Biology (John Wiley &amp; Sons, New York). Additionally, large numbers of tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to marker, for example, a marker nucleotide transcript or marker protein. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the marker mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to marker genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of marker mRNA.

An alternative method for determining the level of marker mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. ScL USA 88:189-193), self sustained sequence replication (Guatelli et al. (1990) Proc. Natl. Acad. ScL USA 87: 1874-1 878), transcriptional amplification system (Kwoh et al. (1989) Proc. Natl. Acad. ScL USA 86:1 173-1 177), Q-Beta Replicase (Lizardi et al. (1988) Bio/Technology 6:1 197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, marker expression is assessed by quantitative fluorogenic RT-PCR (i.e., the TaqMan System). Such methods typically utilize pairs of oligonucleotide primers that are specific for marker. Methods for designing oligonucleotide primers specific for a known sequence are well known in the art.

The expression levels of marker mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection marker expression may also comprise using nucleic acid probes in solution.

In one embodiment of the invention, microarrays are used to detect marker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference.

In one embodiment, the level of marker is determined by measuring an activity of marker as described supra.

In one embodiment, the level of marker is determined by determining a polymorphism in a marker nucleic acid molecule. There are a large number of assay techniques known in the art which can be used for detecting alterations in a polymorphic sequence.

Suitable marker polymorphism can be known or identified as described herein using methods routine to one of skill in the art. For example, given that the human marker coding sequence and flanking sequences are publically available, as are polymorphisms (SNPs) in the marker gene, primers can readily be designed to amplify polymorphic sequences and/or detect marker polymorphisms by one of ordinary skill in the art. For example, a marker sequence comprising a polymorphism (e.g., SNP) can be identified in the NCBI Variation Database (dbSNP) or by homology searching of another database containing human genomic sequences (e.g., using Blast or another program), and the location of the SNP sequence and/or flanking sequences can be determined and the appropriate primers identified and/or designed by one of skill in the art.

In another embodiment, a marker polymorphism(s) is identified and a statistically significant association with the development or the likelihood of developing ulcerative colitis, colorectal cancer or colonization of a subject's gastrointestinal tract with commensal bacteria that cause ulcerative colitis is determined using the methods described herein. It should be noted that it is possible for methods in the art to detect chromosomal variation without specifying an exact SNP site. For example, a tag SNP may be a representative SNP in a region discovered to have high linkage disequilibrium. As such, the methods of the present invention may make use of the named SNPs or other SNPs which reside nearby in the genome or are within the identified regions of linkage disequilibrium.

In one embodiment, analysis of polymorphisms is amenable to highly sensitive PCR approaches using specific primers flanking the sequence of interest. Oligonucleotide primers corresponding to marker sequences can be prepared by standard synthetic techniques, e.g., using an automated DNA synthesizer. In one embodiment, detection of the polymorphism involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364). In one embodiment, genomic DNA of a cell is exposed to two PCR primers and amplification for a number of cycles sufficient to produce the required amount of amplified DNA.

This method can include the steps of collecting a sample of cells from a patient, isolating nucleic acid (e.g., genomic, DNA) from the cells of the sample, contacting the nucleic acid sample with one or more primers which specifically amplify a subject SNP under conditions such that hybridization and amplification of the sequence occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting polymorphisms described herein.

In one preferred embodiment, detection of single nucleotide polymorphisms (“SNP”) and point mutations in nucleic acid molecule is based on primer extension of PCR products by DNA polymerase. (See, e.g., U.S. Pat. No. 6,972,174, the contents of which are incorporated by reference).

In one preferred embodiment, a polymorphism is detected by primer extension of PCR products, as described above, followed by chip-based laser deionization time-of-flight (MALDI-TOF) analysis, as described in, for example U.S. Pat. No. 6,602,662, the contents of which are incorporated by reference.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, after extraction of genomic DNA, amplification is performed using standard PCR methods, followed by molecular size analysis of the amplified product (Tautz, 1993; Vogel, 1997). In one embodiment, DNA amplification products are labeled by the incorporation of radiolabelled nucleotides or phosphate end groups followed by fractionation on sequencing gels alongside standard dideoxy DNA sequencing ladders. By autoradiography, the size of the repeated sequence can be visualized and detected heterogeneity in alleles recorded. In another embodiment, the incorporation of fluorescently labeled nucleotides in PCR reactions is followed by automated sequencing. (Yanagawa, T., et al., (1995). J Clin Endocrinol Metab 80: 41-5 Huang, D., et al., (1998). J Neuroimmunol 88: 192-8.

In other embodiments, polymorphisms can be identified by hybridizing a sample and control nucleic acids to high density arrays containing hundreds or thousands of oligonucleotides probes (Cronin, M. T. et al. (1996) Human Mutation 7: 244-255; Kozal, M. J. et al. (1996) Nature Medicine 2: 753-759).

In one embodiment, any of a variety of sequencing reactions known in the art can be used to directly sequence marker, or a region surrounding marker and detect allelic variants, e.g., mutations, by comparing the sequence of the sample sequence with the corresponding reference (control) sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA polymer chain probe” and U.S. Pat. No. 5,571,676 entitled “Method for mismatch directed in vitro DNA sequencing”.

In some cases, the presence of a specific polymorphism of marker in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242; Cotton et al. (1988) Proc. Natl. Acad Sci USA 85:4397; Saleeba et al (1992) Methods Enzymol. 217:286-295.

In another embodiment, an allelic variant can be identified by denaturing high-performance liquid chromatography (DHPLC) (Oefner and Underhill, (1995) Am. J. Human Gen. 57:Suppl. A266; O'Donovan et al. (1998) Genomics 52:44-49).

In other embodiments, alterations in electrophoretic mobility is used to identify the type of marker polymorphism. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495; Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl. Acad. Sci USA 86:6230; and Wallace et al. (1979) Nucl. Acids Res. 6:3543). Such allele specific oligonucleotide hybridization techniques may be used for the simultaneous detection of several nucleotide changes in different polylmorphic regions of marker. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077 1080. (Nickerson, D. A. et al., (1990) Proc. Natl. Acad. Sci. (U.S.A.) 87:8923 8927. U.S. Pat. No. 5,593,826 Tobe et al. ((1996) Nucleic Acids Res 24: 3728),

In another embodiment, the single base polymorphism can be detected by using a specialized exonuclease resistant nucleotide, as disclosed, e.g., in Mundy, C. R. (U.S. Pat. No. 4,656,127).

In another embodiment of the invention, a solution based method is used for determining the identity of the nucleotide of a polymorphic site (Cohen, D. et al. (French Patent 2,650,840; PCT Application No. WO91/02087).

An alternative method, known as Genetic Bit Analysis or GBA™ is described by Goelet, P. et al. (PCT Application No. 92/15712).

Several primer guided nucleotide incorporation procedures for assaying polymorphic sites in DNA have been described (Komher, J. S. et al., Nucl. Acids. Res. 17:7779 7784 (1989); Sokolov, B. P., Nucl. Acids Res. 18:3671 (1990); Syvanen, A. C., et al., Genomics 8:684 692 (1990); Kuppuswamy, M. N. et al., Proc. Natl. Acad. Sci. (U.S.A.) 88:1143 1147 (1991); Prezant, T. R. et al., Hum. Mutat. 1:159 164 (1992); Ugozzoli, L. et al., GATA 9:107 112 (1992); Nyren, P. et al., Anal. Biochem. 208:171 175 (1993)). (Syvanen, A. C., et al., Amer. J. Hum. Genet. 52:46 59 (1993)).

The methods described herein may be performed, for example, by utilizing pre-packaged diagnostic kits comprising at least one probe/primer nucleic acid or antibody reagent described herein, which may be conveniently used, e.g., in clinical settings to diagnose patients exhibiting symptoms or family history of a disease or illness involving a polymorphic elements. In addition, a readily available commercial service can be used to analyze samples for the polymorphic elements of the invention.

### V. PHARMACEUTICAL COMPOSITIONS

A pharmaceutical composition comprising a compound of the invention, e.g., a stimulatory or inhibitory molecule of the invention or a compound identified in the subject screening assays, is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will preferably be sterile and should be fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

In one embodiment, the test compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, a modulatory agent of the invention is administered in amount sufficient to modulate de novo hepatic lipogenesis, e.g., such that at lest one indicator of de novo hepatic lipogenesis is brought within normal levels. Such indicators may be measured by analyzing serum lipid levels according to methods routine to one of ordinary skill in the art.

This invention is further illustrated by the following examples which should not be construed as limiting. The contents of all references, patents and published patent applications cited throughout this application are hereby incorporated by reference.

### EXAMPLES

The following Materials and Methods were used in the Examples:

### Materials and Plasmids.

Tunicamycin (Tm), wortmannin, 3-methyladenine, and MG-132 were purchased from Calbiochem EMB Bioscience Inc. Cell medium, EBSS media and antibiotics were obtained from Life Technologies (Maryland, USA). Fetal calf serum was obtained from Atlanta Biologicals. Hoechst, lysotraker, and Acridine orange were purchased from Molecular Probes. All transfections were performed using the Effectene reagent (Quiagene). DNA was purified with Quigene kits.

pEGFP-poly(Q) ii and pEGFP-poly(Q)₇₂ were prepared as described before (Kouroku et al., 2002. Hum. Mol. Genet. 11, 1505-1515). SOD1-EGFP expression vectors were described before (Turner et al., 2005. Curr. Mol. Med. 6, 79-86). Primers were designed to introduce a SalI site to allow subcloning SOD1 mutants into pEGFP-N1 (Clontech, Palo Alto, Calif.) and to remove the SOD1 translation stop codon. Mutants were generated via site directed mutagenesis of the SOD1^{WT} template using the Quik Change kit (Stratagene, La Jolla, Calif.). LC3-EGFP expression vectors were described before, where the rat LC3 cDNA was cloned into the B gill and EcoRI sites of the pEGFP-C1 vector (Clontech laboratories) (Kabeya et al., 2000. EMBO J. 19, 5720-5728). LC3-dsRED expression vector was generated by subcloning a 800 bp DNA fragment containing the rat LC3 cDNA excised with BglII and EcoRI enzymes from pEGFP-LC3 and cloned in frame downstream of the dsRed gene into similarly digested pDsRed2-C1 vector (Clontech) as described in (Tasdemir et al., 2007). α-synuclein-EGFP mutants were previously described (Pandey et al., 2006. Exp. Neurol. 197, 515-520). The A30P and E46K mutants were generated through polymerase chain reaction (PCR) and cloned into pcDNA 3.1 (Invitrogen) vector. Then, GFP fusion proteins of each of the constructs were generated by in-frame cloning of AS constructs in pEGFP-N1 (Clontech) vector. XBP-1s expression vector was previously described (Iwakoshi et al., 2003. Nat. Immunol. 4, 321-329), were the cDNA was obtained from NIH3T3 cells treated with tunicamycin. Then XBP-1s cDNA was cloned into the pcDNA.3 vector between the HindIII and ApaI sites.

### Animal Experimentation.

XBP-1^{flox/flox} mice were crossed with mice expressing Cre recombinase under the control of the Nestin promoter (NesCre/+) to achieve deletion of XBP-1 in the nervous system (XBP-1^{Nes-/-}) (Hetz et al., 2008. Proc. Natl. Acad. Sci. U.S. A 105, 757-762). The ALS model, SOD1^{G86R} transgenic mice (the equivalent of human SOD1^{G85R}) which were produced in the FVB/N strain (strain FVB-Tg(Sod1-G86R)M1Jwg/J, The Jackson Laboratory), were employed as an ALS model. The expression of the SOD1 mutant gene is driven by the endogenous SOD1 promoter. As shown in FIG. 1A, the overexpression levels of mutant SOD1 are very low compared with other transgenic mice such as SOD1^{G93A}, decreasing the possible non-specific effects of overexpression. In addition, SOD1^{G86R} encodes an enzyme with low SOD activity, and thus expression of the altered enzyme does not significantly affect overall SOD1 cellular activity when added to the genome in the presence of two wild-type parental genes. All animal experiments were performed according to procedures approved by the Institutional Review Board's Animal Care and Use Committee of Harvard School of Public Health (approved animal protocol 04137) and the Faculty of Medicine of the University of Chile (approved protocol CBA # 0208 FMUCH). Third generation of XBP-1^{Nes-/-}/SOD1^{G86R} mice were used to expand the colony and obtain experimental groups. Disease onset was determined by visual observation of the appearance of abnormal limb-clasping, slight tremor felt in one of the hind-limbs, wobbly gait and the first signs of paralysis in one hind-limb. End disease stage was determined as the time at which an animal can no longer right itself within 30 s after being placed on its back.

### Behavioral Analysis.

Several assays were employed to monitor SOD1^{G86R} performance including rotarod, grip strength and the inverted grill.

RotaRod (Model LE8500, Panlab SL). Animals were trained at 5-8 weeks of age by acclimating to the rotor at 5 RPM, increasing to 10 RPM over a period of five minutes. Training occurred three times over a five day period, with the last session also including a ten second acclimation followed by a one minute progressive increase from 5-60 RPM used as experimental conditions. After training, animals were tested using this method once a week in triplicate allowing 15 seconds between trials. Scores were measured from the start of increase to the point when animals were unable to maintain upright on the rotor. Inability to maintain on rotor was denoted as zero, and animals able to maintain until full speed were given a score of 60.

Grip Strength (Ugo Basile, #47105). Animals were allowed to attach by the front paws to the force transducer using a trapeze-bar, then pulled under constant force. Peak measurements were acquired weekly in triplicate and averaged for each session. Inability to grip was denoted as zero.

Inverted Grill: Mice were attached upside down to a 5 mm spaced wire mesh panel for one minute each week. The inability to attach for less than a minute was noted.

### Tissue Analysis.

To monitor SOD1 pathogenesis in vivo, animals were euthanized and tissue collected for histology at different time points depending on the analysis required. Spinal cord tissue was processed for immunohistochemistry using standard procedures. Apoptotic cells in the ventral horn were quantified by using the TUNEL assay (Promega). Motoneurons were also directly visualized with an anti-ChAT or anti-NeuN (Chemicon) staining. In addition, staining of LC3 and lysosomes were performed with anti-LC3 (Cell Signaling Technology) and anti-LAMP-2 (Developmental Studies Hybridoma Bank). Confocal microscopy was used to acquire images and then analysis was performed using the IP lab v 4.04 software (Beckon and Dickenson).

### Western Blot Analysis of Spinal Cord Extracts.

One cm lumbar spinal cord tissue was collected and homogenated in RIPA buffer (20 mM Tris pH 8.0, 150 mM NaCl, 0.1% SDS, 0.5% DOC, 0.5% triton X-100) containing a protease inhibitor cocktail (Roche, Basel, Switzerland) by sonication. Protein concentration was determined by micro-BCA assay (Pierce, Rockford, Ill.). The equivalent of 30-50 μg of total protein was loaded onto 4-12, 7.5, 12 or 15% SDS-PAGE minigels (Cambrex Biosciences) depending on the analysis as described before. The following antibodies and dilutions were used: anti-Grp78/Bip, anti-PDI, 1:2,000 (StressGene, San Diego, Calif.), anti-XBP-1,1:1,000 (Iwakoshi et al., 2003), anti-GFP 1:1000, anti-Ubiquitin 1:2000, anti-ATF4, anti-Hsp90, anti-CHOP 1:2,000 (Santa Cruz, Calif.); anti-SOD1 1:3000 (Calbiochem), anti-LC3 1:500, anti-Beclin-1 1:2000 (Cell Signaling Technology).

### RNA Extraction and RT-PCR.

Total RNA was prepared from spinal cord tissue homogenated in cold PBS using Trizol (Invitrogen, Carlsbad, Calif.) and cDNA was synthesized with SuperScript III (Invitrogen, Carlsbad, Calif.) using random primers p(dN)₆ (Roche, Basel, Switzerland). Quantitative real-time PCR reactions employing SYBR green fluorescent reagent were performed in an ABI PRISM 7700 system (Applied Biosystems, Foster City, Calif.). The relative amounts of mRNAs were calculated from the values of comparative threshold cycle by using β-actin as control. Primer sequences were designed by Primer Express software (Applied Biosystems, Foster City, Calif.). Real time PCR was performed as previously described (Lee et al., 2005. EMBO J. 24, 4368-4380) using the following primers: grp78/bip 5′-TCATCGGACGCACTTGGAA-3′ and 5′-CAACCACCTTGAATGGCAAGA-3′; grp58 5′-GAGGCTTGCCCCTGAGTATG-3′ and 5′-GTTGGCAGTGCAATCCAC C-3′; Chop/gadd153 5′-GTCCCTAGCTTGGCTGACAGA-3′ and 5′-TGGAGAGC GAGGGCTTTG-3′; xbp-1 5′-CCTGAGCCCGGAGGAGAA-3′ and 5′-CTCG AGCAGTCTGCGCTG-3′; pdi 5′-CAAGATCAAGCCCCACCTGAT-3′ and AGTTCGCCCCAACCAGTACTT; erdj4 5′-CCCCAGTGTCAAACTGTACCAG-3′ and 5′-AGCGTTTCCAATTTTCCATAAATT-3′; edem 5′-AAGCCCTCTGGAACTTGCG-3′ and 5′-AACCCAATGGCCTGTCTGG-3′; sec61a 5′-CTATTTCCAGGGCTTCCGAGT-3′ and 5′-AGGTGTTGTACTGGCCTCGGT-3′; herp 5′-CATGTACCTGCACCACGTCG-3′ and 5′-GAGGACCACCATCATCCGG-3′; actin 5′-TACCACCATGTACCCAGGCA-3′ and 5-′ CTCAGGAGGAGCAATGATCTTGAT-3′; wfs-1 5′-CCATCAACATGCTCC CGTTC-3′ and 5′-GGGTAGGCCTCGCCAT-3′; grp94, 5′-TGTATGTACGCCGCGTATTCA-3′ and 5′-TCGGAATCCACAACACCTTTG-3′.

XBP-1 mRNA Splicing Assays.

XBP-1 mRNA splicing assay was performed as previously described (Lee et al., 2003a. Proc. Natl. Acad. Sci. U.S. A 100, 9946-9951; Iwakoshi et al., 2003. Nat. Immunol. 4, 321-329). In brief, PCR primers 5′-ACACGCTTGGGAATGGACAC-3′ and 5′-CCATGGGAAGA TGTTCTGGG-3′ encompassing the spliced sequences in xbp-1 mRNA were used for the PCR amplification with AmpliTaq Gold polymerase (Applied Biosystem, Foster City, Calif.). PCR products were separated the by electrophoresis on a 2.5% agarose gel (Agarose-1000 Invitrogen, Carlsbad, Calif.) and visualized by ethidium bromide staining. Alternatively, in spinal cord extracts an alternative method was employed to monitor XBP-1 mRNA splicing. PCR using the sense primer mXBP1.3S (5′-AAACAGAGTAGCAGCGCAGACTGC-3′) and antisense primer mXBP1.2AS (5′-GGATCTCTAAAACTAGAGGCTTGGTG-3′) amplified a 600-bp cDNA product encompassing the IRE1α cleavage sites. The fragment was further digested by PstI to reveal a restriction site that is lost after IRE1-mediated cleavage and splicing of the mRNA (Calfon et al., 2002. Nature 415, 92-96).

### Knockdown of UPR Components in Motoneurons.

Stable motoneuron cell lines with reduced levels of XBP-1, IRE1α, Beclin-1 and EDEM were generated using methods previously described (Hetz et al., 2007) by targeting the respective mRNA with shRNA using the lentiviral expression vector pLKO.1 and puromycin selection. As control empty vector or a shRNA against the luciferase gene were employed. Constructs were generated by The Broad Institute (Boston, USA) based on different criteria for shRNA design (see broad.mit.edu/genome_bio/trc/rnai.html). A total of five different constructs for each gene were screened and the most efficient one was selected for further studies. Targeting sequences identified for the mouse XBP-1, IRE1α, Beclin-1 and EDEM mRNA are 5′-CCATTAATGAACTCATTCGTT-3′,5′-GCTCGTGAATTGATAGAGAAA-3′,5′-GCGGGAGTATAGTGAGTTTAA-3′,5′-CCATTAATGAACTCATTCGTT-3′,5′-GCTCGTGAATTGATAGAGAAA-3′,5′-GCGGGAGTATAGTGAGTTTAA-3′, 5′-CCATATCATATCTGTGGACAA-3′ and 5′-GCCCTTAAAGAGCATCTACAT-3′, respectively.

The NSC34 cell model was selected for this study because it has several valuable characteristics of motor neurons (Cashman et al., 1992. Dev. Dyn. 194, 209-221), which include the ability to induce contraction in co-cultured muscle cells; the expression of neurofilaments; the generation of action potentials and the synthesis, storage and release of acetylcholine. In addition, NSC34 cells induce acetylcholine receptor clusters on co-cultured myotubules and they are sensitive to mutant SOD1 neurotocixity (Turner et al., 2005. J. Neurosci. 25, 108-117). All experiments were performed in the presence of 2% FBS to induce differentiation of NSC34 cells that is corroborated by the observation of axonal projections in a subpopulation of cells. Cells were grown in the presence of 3 μg/ml puromycin to maintain selective pressure.

### Assays for Mutant SOD1 Aggregation and Detection of Intracellular Inclusions.

Assays using the transient expression of human SOD1^{WT} and the mutants SOD1^{G93A} and SOD1^{G85R} as EGFP fusion proteins were developed. These constructs were employed to visualize and quantify the formation of intracellular SOD1 inclusions in living cells by fluorescent confocal microscopy. All experiments were performed in complete media containing 2% FBS. SOD1 oligomers were visualized in total cell extracts prepared in RIPA buffer and sonication, and then analyzed by Western blot. Alternatively, post-nuclear cell lysates were prepared in 1% NP-40 in PBS containing protease inhibitors. After solubilization on ice for 30 min, cell nucleus was precipitated by a centrifugation at 3000 rpm for 5 min. Then, cell extracts were centrifuged at 10,000 g for 10 min to collect NP-40 soluble and insoluble material. Pellets were resuspended in Western blot sampler buffer containing SDS.

### Quantification of Autophagy and Cell Viability.

Lysosomes or acidic compartments were visualized with two different assays. Living cells were stained with 200 nM lysotraker or 600 nM Acridine orange for 45 min at 37 C and 5% CO2. Cells were washed three times with cold PBS and then fixed for 30 min with 4% formaldehyde on ice, then maintained in PBS containing 0.4% formaldehyde for visualization on a confocal microscope. Autophagosomes were visualized after the expression of LC3-EGFP (Kabeya et al., 2000. EMBO J. 19, 5720-5728) or LC3-dsRED after the transient transfection of low amount of DNA (⅓ amount of recommended concentrations by transfection kit). As control experiments, cells were treated with 10 mM 3-MA autophagy inhibitor. All quantifications were performed at least in a total of 150 cells performed in duplicate for each independent experiment.

Autophagosomes were also visualized by transmission electron microscopy. Cells were fixed with 1.25% formaldehyde, 2.5% glutaraldehyde, 0.03% picric acid in 100 mM sodium cacodylate buffer. After washing with 100 mM sodium cacodylate buffer, tissues were treated for 1 h with 1% osmium tetroxide and 1.5% potassium ferrocyanide, and then 30 min with 0.5% uranyl acetate in 50 mM maleate buffer, pH 5.15. After dehydration in ethanol, cells were treated for 1 h in propylenoxide and then embedded in Epon/Araldite resin. Ultrathin sections were collected on electron microscope grids and observed by using a JEOL 1200EX transmission electron microscope at an operating voltage of 60 kV.

Cell viability was be monitored using the MTT assay or propidium iodide staining (Hetz et al., 2006. Curr. Mol. Med. 6, 37-43; Hetz et al., 2007. Cell Death. Differ. 14, 1386-1389).

### Example 1

### Activation of the UPR in Sporadic and Familial ALS

To define the contribution of the UPR to ALS, the expression levels of different UPR markers in post-mortem spinal cord samples from sALS and fALS patients was analyzed. Very marked expression of the UPR transcription factors XBP-1s and ATF4 in both sALS and fALS cases (FIG. 1A) was observed. Consistent with this result, classical UPR target genes such as Grp94, Grp78/BiP and Grp58 were induced (although to variable degrees) in the disease but not control samples (FIG. 1A). Other stress component proteins including eIF2α and phosphorylated ERK were also upregulated in ALS and fALS spinal cord (FIG. 1A). Consistent with these results, increased expression of the XBP-1s target EDEM 1 was observed. These results indicate that ER stress responses are active in both sALS and fALS.

In order to investigate the functional role of the UPR in ALS, the expression levels of different UPR markers were analyzed in two different mutant SOD1 transgenic models expressing human SOD1^{G93A} or mouse SOD1^{G86R}, the equivalent of human SOD1^{G85R} (FIG. 1B). The latter model uses the SOD1 endogenous promoter to express the enzymatically inactive SOD1^{G86R}, and has the advantage of expressing very low levels of the protein, which minimizes non-specific effects due to overexpression. The upregulation of three UPR markers (BiP, PDI and CHOP) was observed in the spinal cord of symptomatic mice when compared to control animals (FIG. 1B). In addition, a clear activation of XBP-1 mRNA splicing was observed in SOD1^{G86R} transgenic mice (FIG. 1C). In agreement with this, XBP-1s target genes herp, erdj4, wfs-1, Sec61 and Grp58 (Hetz et al., 2008. Proc. Natl. Acad. Sci. U.S. A 105, 757-762; Shaffer et al., 2004. Immunity. 21, 81-93; Lee et al., 2003b. Mol. Cell Biol. 23, 7448-7459) were significantly upregulated in symptomatic mice (FIG. 1D). In addition, other UPR-related genes such as bip, erol and chop were induced at the transcriptional level (FIG. 1D). It is important to highlight that the significant differences described in these experiments are observed in a total tissue extract where the UPR may well be occurring in a non-synchronized subpopulation of cells in the spinal cord. In summary, a clear engagement of ER stress responses in fALS models in vivo was observed consistent with previous reports.

### Example 2

### XBP-1 Deficiency Prolongs Lifespan in SOD1 Transgenic Mice

To study the contribution of the UPR to fALS in vivo, an XBP-1 conditional knockout model was generated in which xbp-1 was deleted in the nervous system using the Nestin-Cre system (XBP-1^{Nes-/-}) (Hetz et al., 2008. Proc. Natl. Acad. Sci. U.S. A 105, 757-762). These mice developed normally and did not show any spontaneous disease. XBP-1^{Nes-/-} mice were cross-bred with SOD1^{G86R} transgenic mice to evaluate the role of XBP-1 in disease onset and animal survival. Surprisingly, XBP-1 deficiency resulted in an increase in life span of SOD1^{G86R} mice from a median survival of 110 days to 120 days (FIG. 2A). Of particular interest, the protective effects of XBP-1 deficiency were markedly accentuated in female animals (FIG. 2B), as evidenced by a highly significant increase of 21 days in life span (p=0.0019), with an average survival of 110 and 132 days for XBP-1^{WT}- and XBP-1^{Nes-/-}-SOD1^{G86R} mice, respectively. These results correlated with a 30% decrease in apoptosis in the ventral horn of the spinal cord of XBP-1^{Nes-/-}/SOD1^{G86R} mice compared with control animals (FIG. 2C, p=0.03). Analysis of disease onset of cross-bred mice demonstrated that deletion of xbp-1 in SOD1^{G86R} mice slightly delayed disease onset. The appearance of other disease symptoms in SOD1^{G86R} such as decrease in motor performance, muscle strength and body weight were also delayed in XBP-1 deficient mice. These results were completely unexpected, as the prediction was that impairment of the IRE1α/XBP-1-dependent adaptive response would accelerate neuronal dysfunction, therefore decreasing the life span and disease severity of fALS mouse models.

### Example 3

### Targeting IRE1α and XBP-1 Decreases Mutant SOD1 Aggregation and Toxicity

To better define the role of XBP-1 and IRE1α in SOD1 pathogenesis, the expression of xbp-1 and IRE-1 alpha was reduced in NSC34 cells, a well established motoneuron cell line, using lentiviral delivery of small hairpin RNAs (shRNA) (termed shXBP-1 and shIRE1 cells, respectively). This method led to an efficient decrease of XBP-1s expression in cells undergoing experimental ER stress triggered by tunicamycin treatment (FIGS. 3A and B). In addition, knockdown of XBP-1 had a functional effect since the transcriptional upregulation of many UPR-target genes was significantly decreased (FIG. 3C). XBP-1 independent genes such as chop and grp94 were not affected. To monitor SOD1 misfolding in these cells, human SOD1^{WT} or the mutants SOD1^{G93A} and SOD1^{G85R} were transiently expressed as EGFP fusion proteins and the accumulation of intracellular SOD1 inclusions was examined by fluorescent microscopy (FIG. 3D). Interestingly, a nearly 50% reduction in the number of cells harboring SOD1^{G93A} and SOD1^{G86R} intracellular inclusions was observed in shXBP-1 and shIRE1α NSC34 cells (FIGS. 3D and 8B). To complement these observations, the generation of high molecular weight and detergent insoluble SOD1 species was monitored by Western blot analysis. Knocking-down XBP-1 or IRE1α drastically decreased the generation of toxic SOD1^{G93A} and SOD1^{G85R} protein aggregates (FIGS. 3E and 8C). Similar results were obtained when IRE1a and XBP-1 knockdowns were performed in Neuro2a cells. The effects of XBP-1s gain of function on mutant SOD1 aggregation was also investigated. After co-transfection of an XBP-1s expression vector with SOD1^{G93A} or SOD1^{G85R} increased aggregation of mutant SOD1 and augmented generation of intracellular inclusions was observed (FIGS. 3F and G). These data are in agreement with the in vivo data presented above. In both cases, the results were completely unexpected because the prediction was that impairment of the UPR by shRNA-mediated targeting would increase SOD1 aggregation due to improper ER function. Taken together, these results show that XBP-1 levels determine the basal levels of disease-related mutant protein aggregation in motoneuron cells.

### Example 4

### Targeting IRE1α/XBP-1 Upregulates Autophagy as does ERAD Impairment

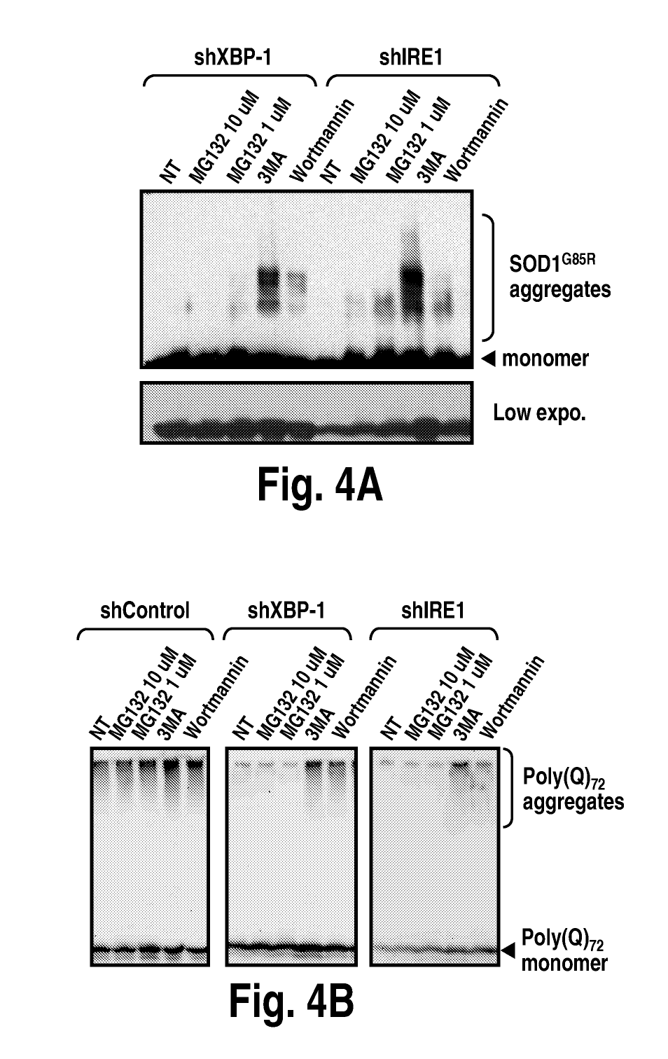

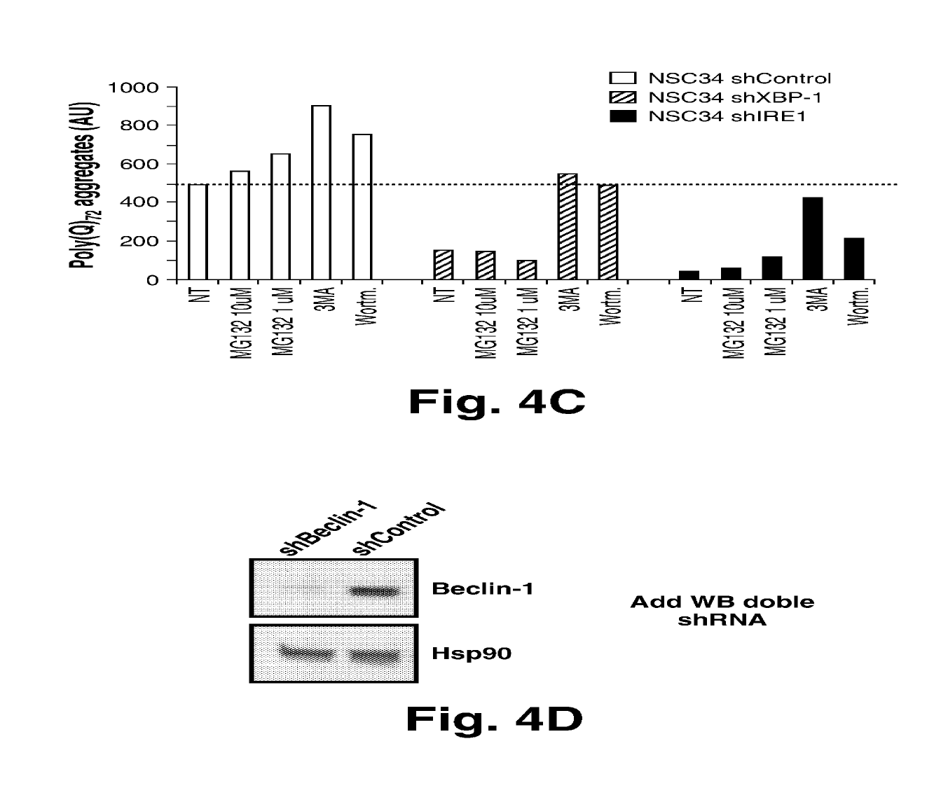

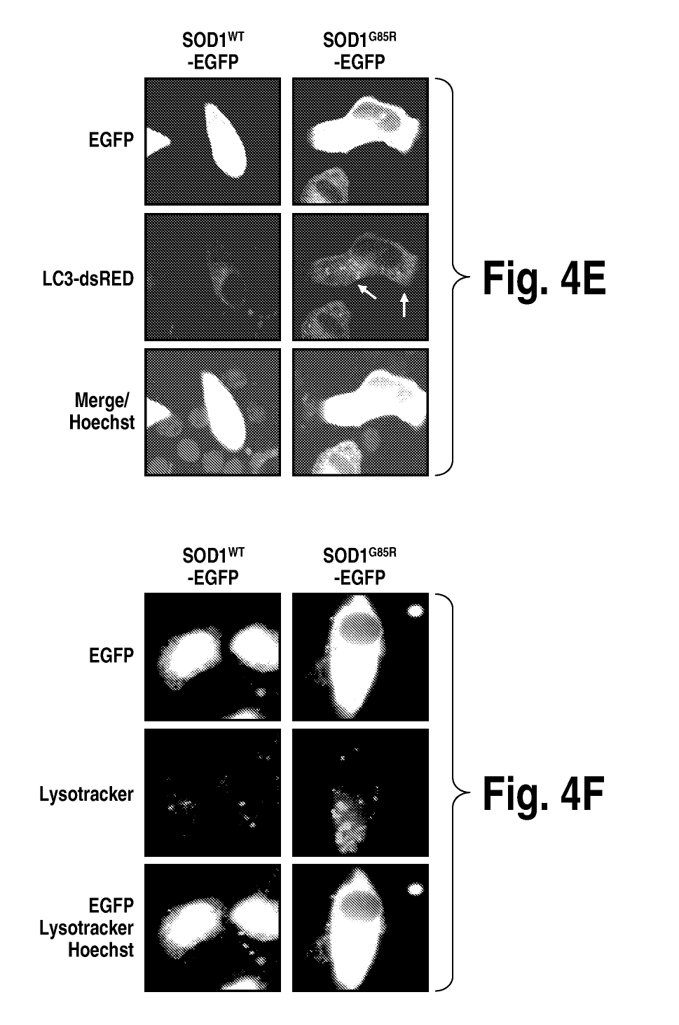

Diminished SOD1 aggregation in knockdown NSC34 cells might be explained by XBP-1 deficiency-mediated and/or upregulation of protein degradation pathways involved in mutant SOD1 clearance. Both the proteasome and autophagy pathways have been shown to mediate mutant SOD1 degradation in vitro (Kabuta et al., 2006. J. Biol. Chem. 281, 30524-30533) and a direct connection between the UPR and autophagy has been recently suggested by cellular studies (reviewed in Hoyer-Hansen and Jaattela, 2007. Cell Death. Differ. 14, 1576-1582). Autophagy is a “large-scale” cellular degradation process for proteins and damaged organelles (Maiuri et al., 2007. Nat. Rev. Mol. Cell Biol. 8, 741-752). Macroautophagy, a process generally referred to as autophagy, involves the formation of double-membrane-bound structures known as autophagosomes. These fuse with lysosomes to form autophagolysosomes, whose contents are then degraded by acidic lysosomal hydrolases. In order to define the contribution of the proteasome and autophagy in SOD1 clearance, shRNA NSC34 cells were treated with a proteasome inhibitor (MG-132) and PI3-kinase inhibitors (3-methyladenine [3-MA], and Wortmannin) which block an early step controlling autophagosome formation to compare the effects on SOD1 detergent insolubility. Blocking PI3-kinase resulted in more SOD1 aggregation than proteasome inhibition, with recovery of mutant SOD1 aggregation in knockdown cells (FIGS. 4A and 9A). These results were also confirmed by knocking down Beclin-1/ATG6, the first identified mammalian gene product shown to be essential for initiation of autophagy (Maiuri et al., 2007. Nat. Rev. Mol. Cell Biol. 8, 741-752; Levine and Yuan, 2005. Clin. Invest 115, 2679-2688), and found increased levels of mutant SOD1 in shXBP-1 NSC34 cells (FIG. 4D). Similar results were observed when poly-(Q)₇₂ aggregation was assessed, suggesting that IRE1α/XBP-1 deficiency has a broad impact in disease-related mutant protein aggregation.

LC3 (also known as ATG8) is a commonly used marker of autophagy that specifically localizes to autophagosomes. To define the effect of SOD1 on the levels of autophagy, SOD1^{WT} and SOD1^{G85R} were co-transfected with a LC3-dsRED fusion protein to monitor LC3 redistribution. As shown in FIG. 4E, mutant SOD1 specifically increased the levels of LC3-containing autophago some dots, consistent with previous findings (Kabuta et al., 2006). Based on the marked effects of 3-MA on SOD1 aggregation, the degree of co-localization between SOD1 mutants and the lysosomal/acidic compartments was determined by confocal microscopy. Similarly, increased lysosome content was detected after visualization of shXBP-1 cells with lysotraker, accridine orange staining, or electron microscopy. These studies were complemented by measuring the co-localization of mutant SOD1 aggregates with DQ-BSA positive vacuoles, a dye that stains active proteolysis at the lysosome. To measure the functional degredation of mutant SOD1 by the lysosomal pathway, shXBP-1 cells were treated with a cocktail of lysosomal inhibitors (bafilomycin A1 and the protease inhibitors pepstatin and E64d). Using this approach, an enhanced accumulation of SOD1 aggregates and inclusions in shXBP-1 cells was seen after inhibiting lysosomal activity.

These results were extended by knocking down ATG5, a major autophagy regulator in the nervous system (Hara et al., 2006). shXBP-1 cells were transduced with shRNA lentiviruses against the atg5 mRNA, that reduced its mRNA levels by ˜70%. A significant increase in the levels of mutant SOD1 aggregation was observed in shXBP-1 cells when ATG5 expression was knocked down, reverting the phenotype of XBP-1 deficiency. Similar results were obtained when we targeted the expression of Beclin-1/ATG6, the first identified mammalian gene product shown to regulate autophagy (Liang et al., 1999) (reviewed in (Mizushima et al., 2008), in shXBP-1 cells. Thus, our results indicate that XBP-1 deficiency increases mutant SOD1 clearance due to autophagy-mediated degradation.

It was observed that there are two different phenomena in this analysis: (i) there was a strong co-localization between SOD1 mutant inclusions and lysosomes in cells overexpressing mutant SOD1^{G85R} but not SOD1^{WT}, and (ii) cells displaying SOD1 mutant inclusions contained greater numbers of lysosomes than non-expressing cells in the same experiment (FIG. 4F and FIG. 10A).

To determine whether or not the presence of LC3-positive vacuoles is related to augmented autophagy activity rather than to decreased lysosomal fusion/degradative activity, the flow of LC3 through the autophagy pathway using previously described approaches (Klionsky et al., 2008) was monitored. The level of endogenous LC3-II (the active phosphatidylethanolamine conjugated form) was measured under resting conditions in shXBP-1 and control cells in the presence or absence of lysosomal inhibitors. A clear increase in the expression of LC3-II in shXBP-1 cells was observed when compared with control cells. Interestingly, LC3-II levels were further augmented by blocking lysosomal activity, indicating elevated autophagy activity and LC3-flux in shRNA cells. In agreement with this result, LC3-II levels were decreased in shXBP-1 cells when the upstream regulator ATG5 was knocked down in these cells, consistent with the results observed in SOD1 aggregation assays. These data were also corroborated by expressing a tandem monomeric RFP-GFP-tagged LC3, where the LC3 flux into the lysosomal acidic compartment can be followed in living cells in the absence of drug treatment (Klionsky et al., 2008).

In summary, these results show that autophagy may be overactive in cells deficient in IRE1α signaling and that the UPR has an important role in modulating the clearance of abnormal SOD1 aggregates.

Genes related to proteasome-mediated degradation of misfolded proteins through the ER-associated degradation (ERAD) pathway are a major target of XBP-1s (Lee et al., 2003). Through ERAD, misfolded proteins accumulated at the ER are retrotranslocated to the cytosol for degradation by the proteasome. Autophagy has been suggested to act as a second ERAD pathway for degradation of ER-located misfolded proteins (Fujita et al., 2007). The possibility that XBP-1 deficiency may affect ERAD and other related processes, leading to increased basal levels of misfolded proteins at the ER, activating autophagy as a survival mechanism was investigated.

To determine the effects of XBP-1 on ERAD activity in motoneurons, the levels of a classical ERAD substrate, CD3-δ-YFP (Lerner et al., 2007) in shXBP-1 cells under resting conditions were monitored. To monitor the flow of CD3-δ-YFP through the ERAD pathway for proteasome-mediated degradation, experiments were performed after inhibition of translation by cycloheximide treatment in the presence or absence of proteasome inhibitors. Western blot analysis of CD3-δ-YFP expressing cells revealed a marked decrease in its degradation in shXBP-1 cells when compared with control cells. To test the possible involvement of ERAD-related XBP-1 target genes in the upregulation of autophagy EDEM, a key protein in ERAD (Molinari et al., 2003) that is specifically upregulated by XBP-1 under ER stress conditions (Lee et al., 2003) was knocked down. Interestingly, decreasing EDEM levels in NSC34 cells recapitulated the phenotype of XBP-1 silencing, with reduced accumulation of SOD1^{G85R} detergent insoluble species. In addition, elevated numbers of LC3-labeled autophagosomes were observed in these cells. These results suggest that the protective effects of XBP-1 deficiency on SOD1 pathogenesis are related to its essential regulation of ERAD function.

### Example 5

### Targeting of XBP-1 Leads to Autophagy-Mediated Degradation of Huntington's Disease-Related Mutant Protein

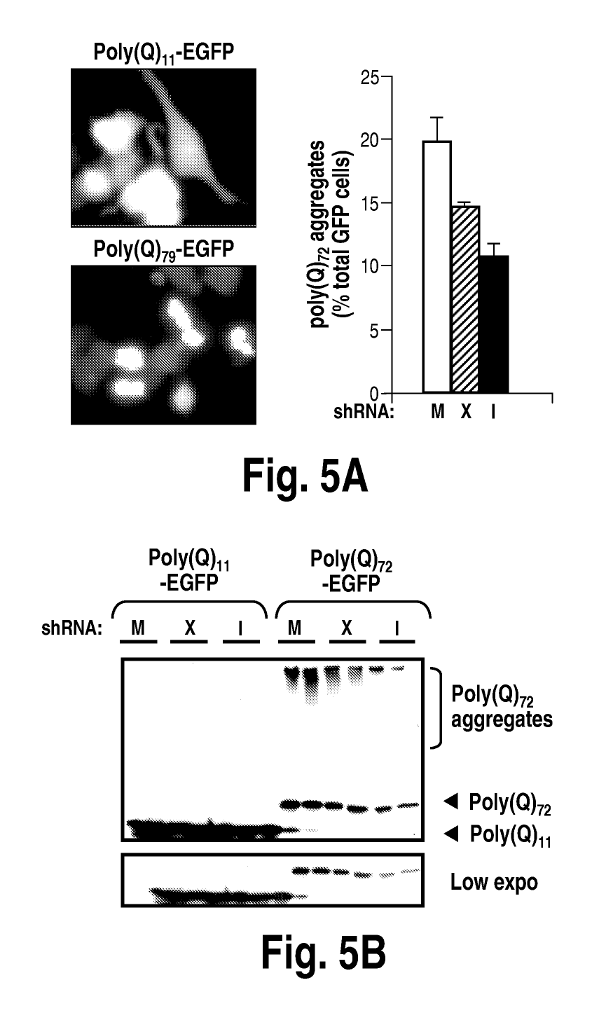

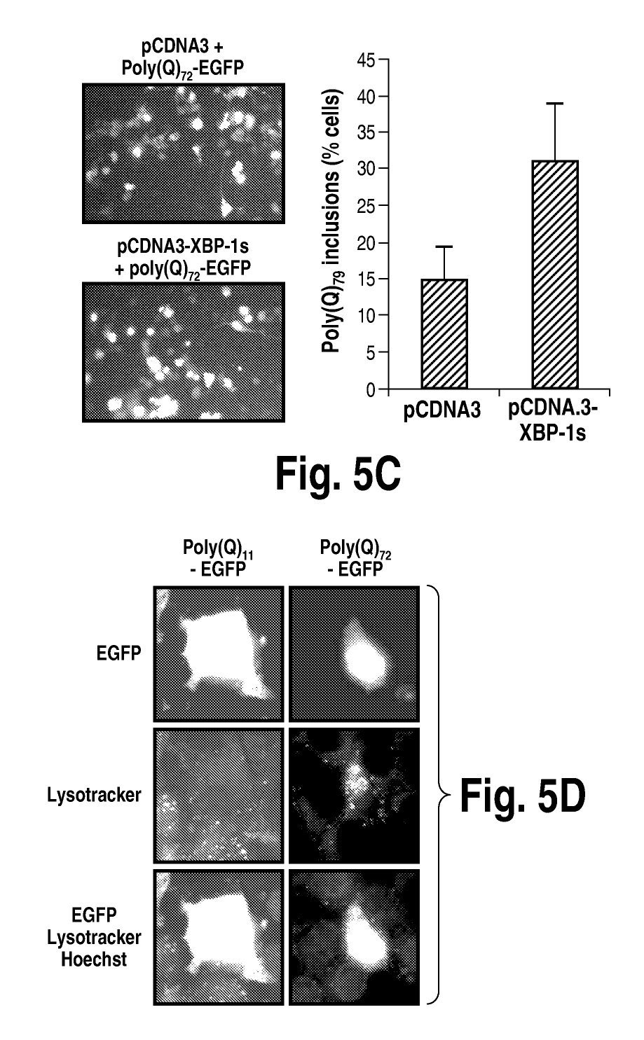

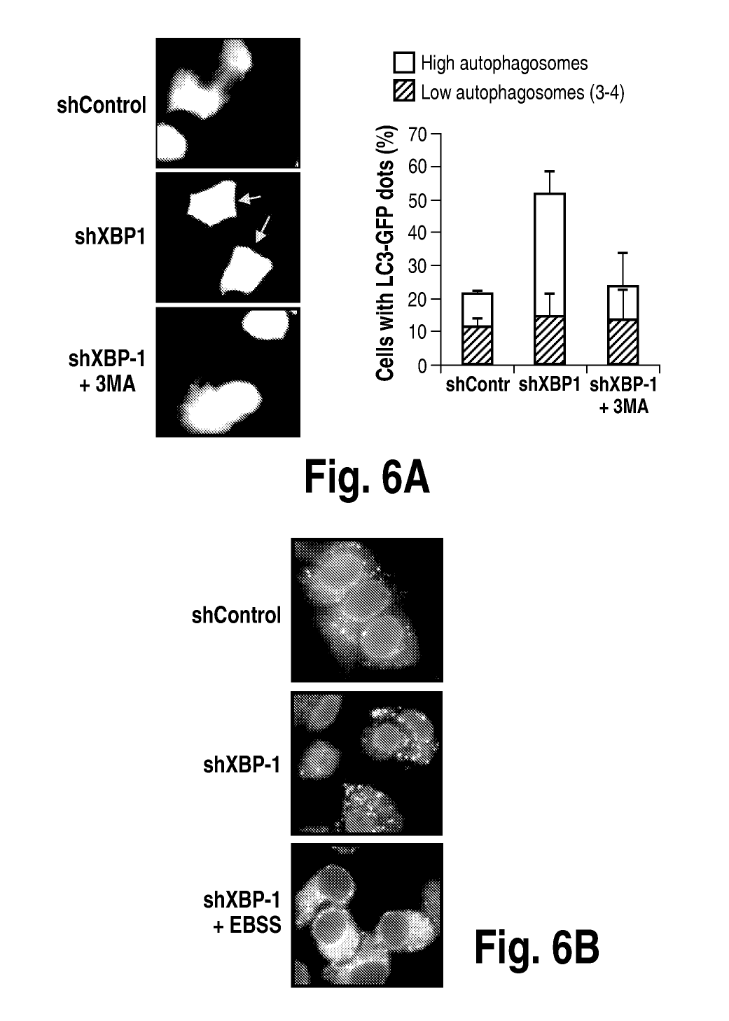

To address the effects of XBP-1 and IRE1α deficiency in other protein misfolding diseases, the levels of Huntington's disease-linked poly-glutamine(Q) aggregation were assessed. A clear decrease in the levels of pathogenic poly-(Q)₇₂ intracellular inclusions was observed a (FIG. 5A and detergent insoluble species (FIG. 5B) in shXBP-1 and shIRE1α cells. These effects were not observed when control poly-(Q)₃₂ peptide was expressed. In addition, XBP-1s expression increased the generation of poly-(Q)₇₂ intracellular inclusion and detergent insoluble species (FIG. 5C). Similar to mutant SOD1, expression of poly-(Q)₇₂ increased lysosome number when compared to cells lacking intracellular inclusions or expressing control peptide (FIG. 5D and FIG. 10B). Interestingly, this phenomenon was not observed when α-synucleinA53T and α-synucleinE46K mutants linked to Parkinson's disease were expressed, (FIG. 10C), suggesting that the increase in autophagy levels by disease-related mutant proteins is specific to certain misfolded proteins. Finally, blockade of autophagy but not proteasome function restored the rate of poly-(Q)₇₂ aggregation in shXBP-1 and shIRE1 cells (FIG. 4B, C and FIG. 9B). Hence IRE1α/XBP-1 deficiency reduces disease-related mutant protein aggregation.

### Example 6

### The Protective Effects of XBP-1 Deletion are Associated with Increased Macroautophagy

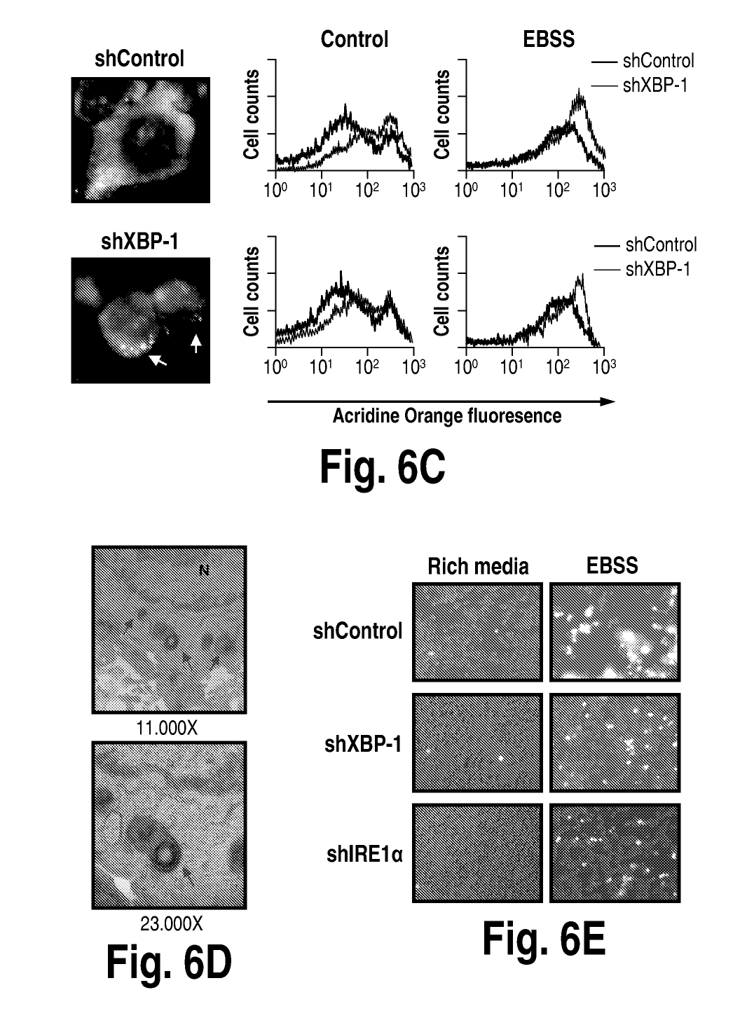

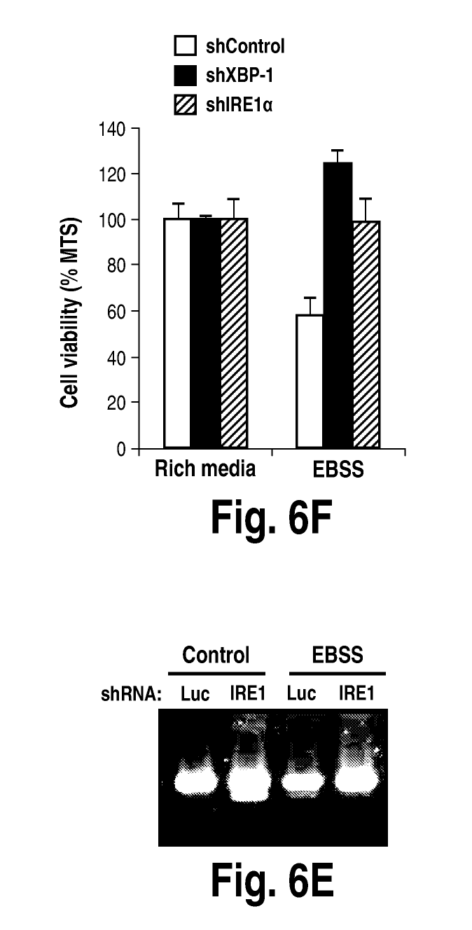

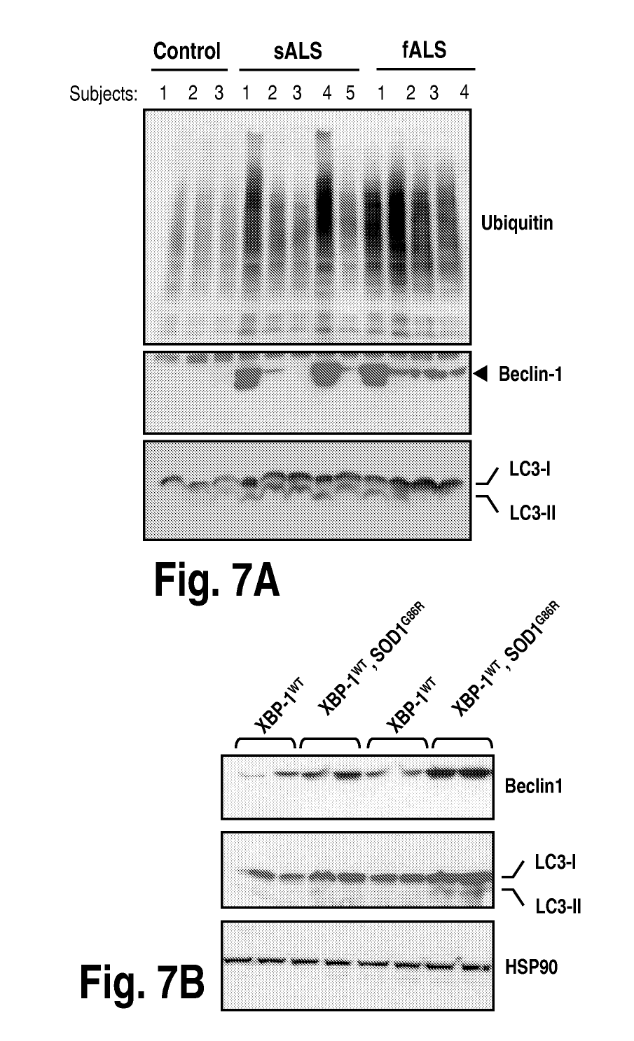

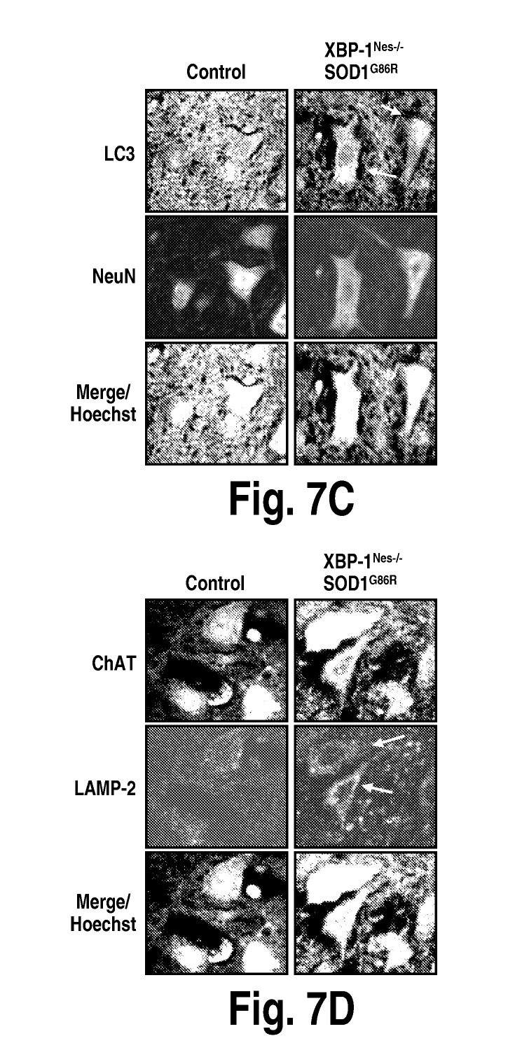

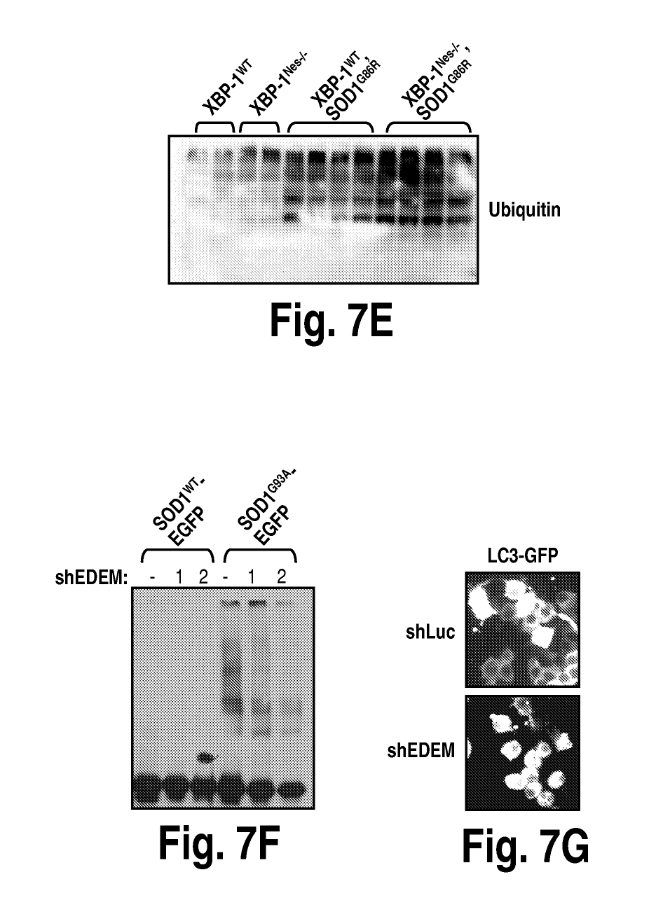

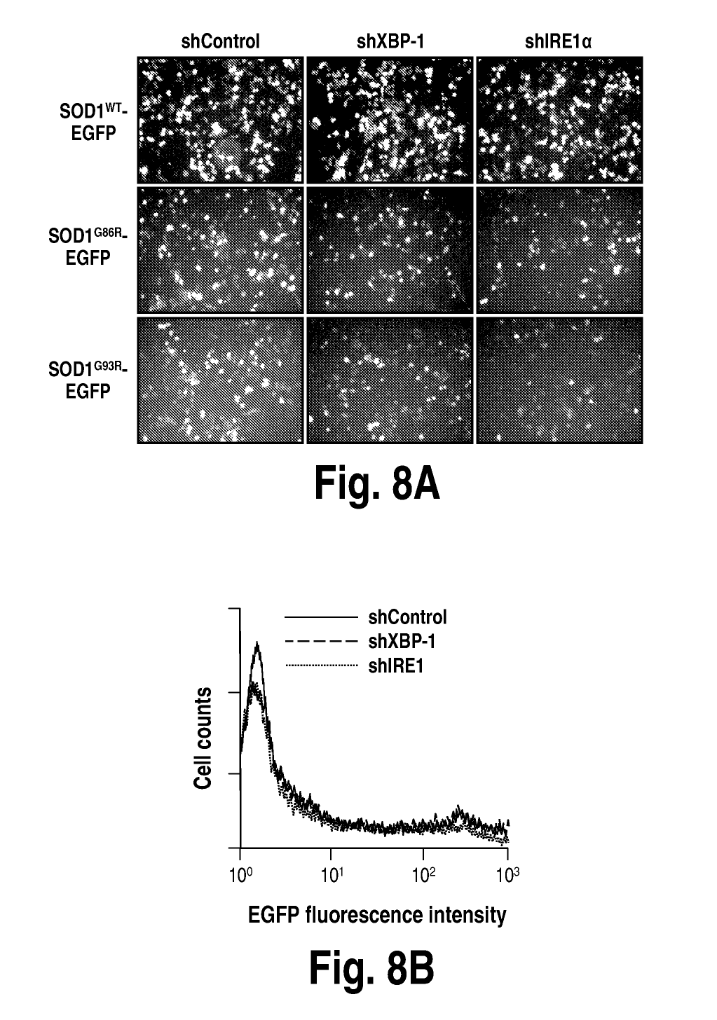

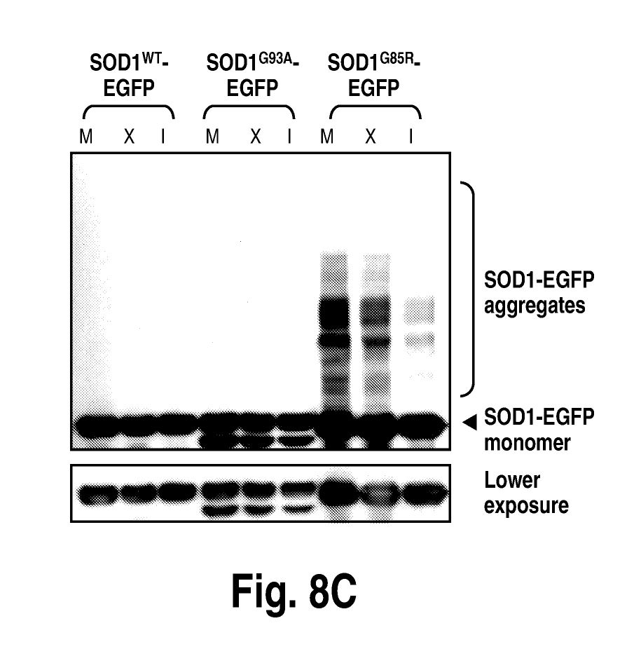

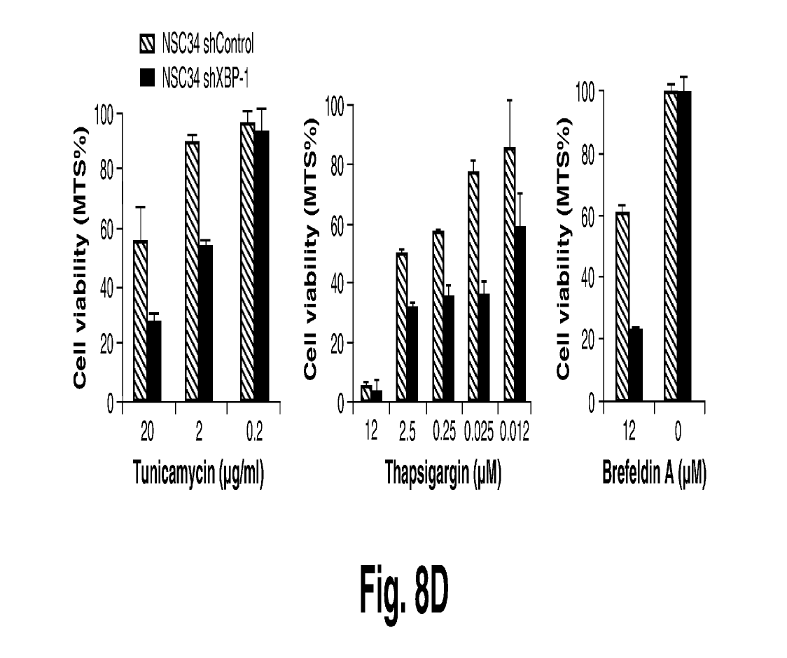

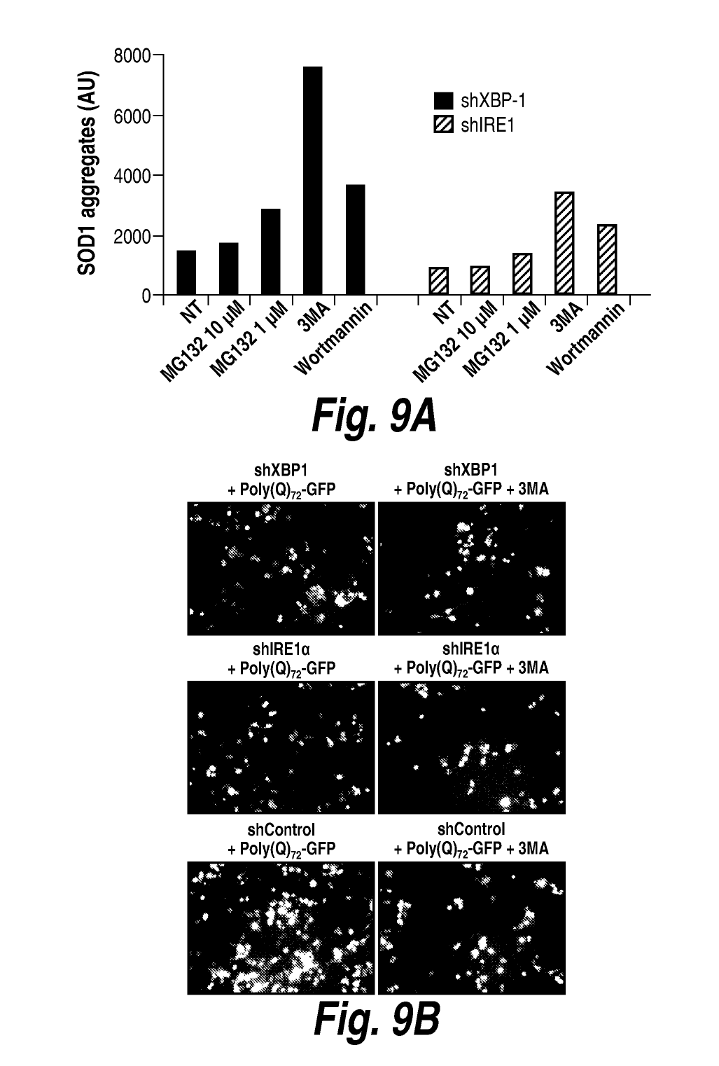

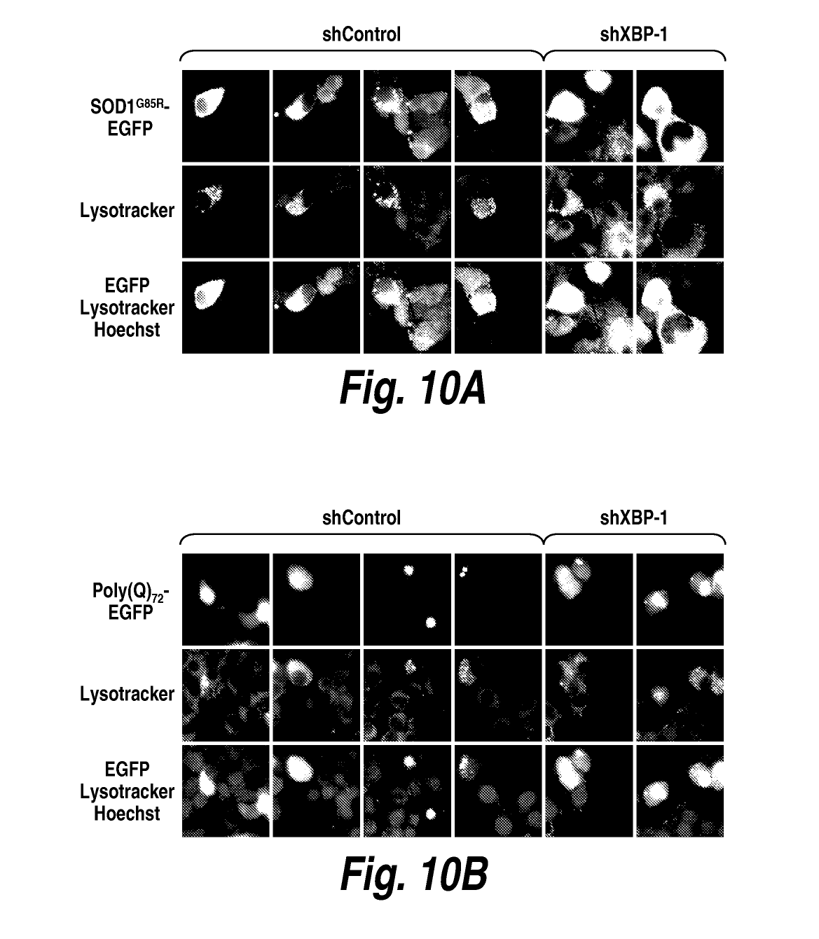

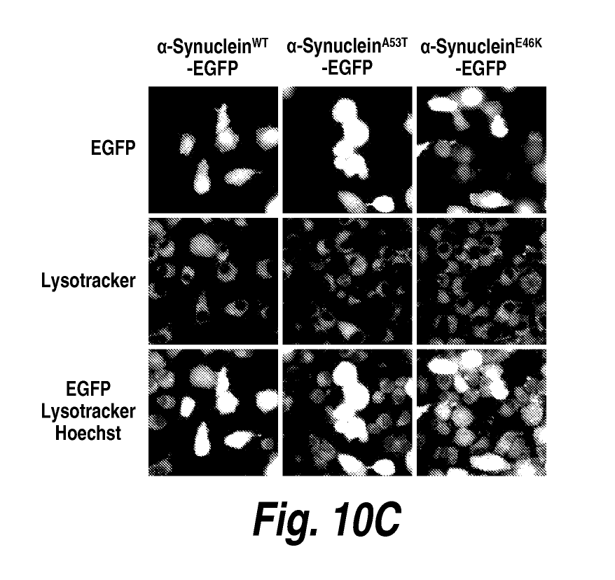

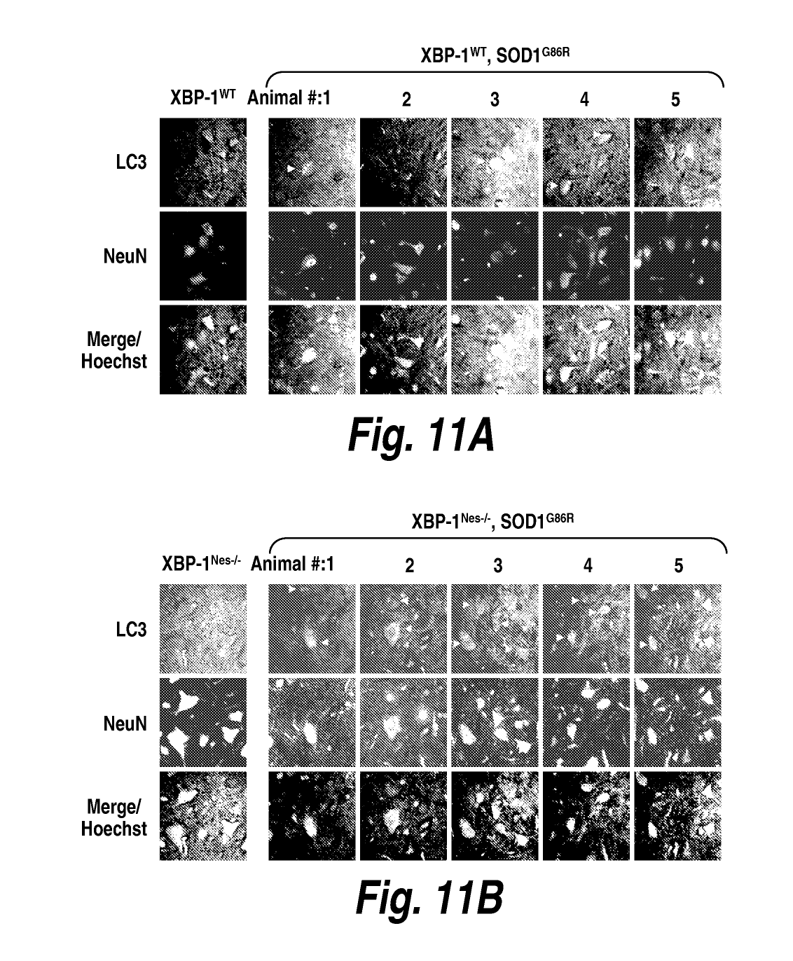

Based on the effects of 3MA on mutant SOD1 aggregation in vivo, autophagosome and lysosome content in shXBP-1 cells under resting conditions were measured in vitro. An LC3-EGFP fusion protein was expressed in shControl and shXBP-1 cells to monitor autophagosome number. A clear increase in the number of cells containing more than four autophagosomes per cell was observed in shXBP-1 cells when compared with control cells and was decreased by 3-MA treatment (FIG. 6A). Similarly, increased lyso some content was detected after visualization of cells with lysotraker (FIG. 6B), that co-localized with LC3-EGFP dots, corroborating the acidic nature of LC3-labeled vesicles. Increased numbers of acidic vesicles were also observed in shXBP-1 cells after acridine orange staining and FACS analysis (FIG. 6C). Finally, electron microscopy analysis revealed increased autophagosome content in XBP-1 deficient NSC34 motoneurons (FIG. 6D).

Because autophagy was originally described as a survival pathway under limiting nutrient conditions, the cellular consequence of increased basal autophagy in IRE1α and XBP-1 deficient NSC34 cells was tested by analyzing their susceptibility to nutrient starvation. Using two independent approaches, it was observed that shXBP-1 and shIRE1α cells were more resistant to starvation-induced cell death, possibly related to a preconditioning effect due to increased basal activation of the autophagy machinery (FIGS. 6E and F). These results completely contrasted with the enhanced sensitivity to ER-stress mediated apoptosis of IRE1α/XBP-1 deficient cells (FIG. 8D). In addition, nutrient starvation led to XBP-1 mRNA splicing, highlighting the presence of cross talk between the UPR and autophagy signaling pathways (FIG. 6G).

Increased autophagosome numbers, LC3 processing and inhibition of mTOR (a repressor of autophagy) were recently observed in the spinal cord of SOD1G93A transgenic mice (Morimoto et al., 2007). To characterize the role of autophagy in sporadic and familial ALS, the levels of LC3 processing (a modification related to its activation) and Beclin-1 levels were determined in human post mortem spinal cord samples. As shown in FIG. 7A, activation of autophagy markers was observed in the majority of samples analyzed. Interestingly, the levels of Beclin-1 induction correlated well with increased amounts of poly-ubiquitinated proteins (FIG. 7A) and the upregulation of ER stress-responsive chaperones in these samples (FIG. 1A). Thus, ER stress and autophagy responses are induced in sALS and fALS cases. To test whether autophagy and XBP-1 deficiency are linked in ALS in vivo, autophagy levels in the spinal cord of symptomatic SOD1^{G86R} mice bred onto a wild type or XBP-1 deficient background were measured. Western blot analysis revealed a clear induction of the autophagy regulator Beclin-1 in the ALS model, which was further increased in XBP-1^{Nes-/-}SOD1^{G86R} animals (FIG. 5B). In addition, the processing of LC3 was evident in SOD1^{G86R}/XBP-1^{Nes-/-} animals (FIG. 5B). In agreement with these results, the number of motoneurons containing multiple LC3-labeled autophagosomes was evident in SOD1^{G86R}/XBP-1^{Nes-/-} mice (N=5 per group) (FIG. 5C). SOD1^{G86R} mice breed on an XBP-1 wild type background showed only a slight increase in the number of autophagosomes when compared with non-transgenic mice (FIG. 11). The majority of L3 positive cells were positive for NeuN staining (greater than 90%). Similarly, increased lysosomal content (detected by LAMP-2 staining) was observed in mutant SOD1 mice, and further increased in SOD1^{G86R}/XBP-1^{Nes-/-} mice (FIG. 5D). Overall, these data indicate that targeting XBP-1 leads to compensatory changes that increase autophagy levels.

To monitor the active engulfment of SOD1 aggregates by autophagy in the spinal cord of XBP-1^{Nes-/-} mice, we performed SOD1 immunogold staining and EM analysis. SOD1 inside autophagosomes was visualized using this method. In addition, co-localization of SOD1 and LC3 in vesicular structures was detected by double immunogold staining of pre-symptomatic XBP-1^{Nes-/-}-SOD1^{G86R} mice. Consistent with increased autophagy levels in XBP-1^{Nes-/-} mice and the co-localization of SOD1 with autophagosomal structures, analysis of female animals at the pre-symptomatic stage revealed almost complete elimination of mutant SOD1 aggregates in spinal cord of some XBP-1^{Nes-/-}-SOD1^{G86R} animals when compared with littermate control female mice. Taken together, these data indicate that XBP-1 deficiency leads to increased autophagy, associated with augmented mutant SOD1 clearance and prolonged life span.

A clear augmentation of polyubuiquitinated proteins in sALS and fALS spinal cord samples was observed. One of the main targets of XBP-1s are genes related to proteasome degradation of misfolded proteins through the ERAD pathway (Lee et al., 2003b). Under ER stress conditions, EDEM is specifically upregulated by XBP-1, a crucial gene regulating ERAD (Molinari et al., 2003; Oda et al., 2003). Therefore, XBP-1 deficiency may affect ERAD, leading to increased basal levels of poly-ubiquitinated proteins, which may be the signal triggering an adaptive upregulation of autophagy as previously suggested (Pandey et al., 2007c). Upon examination of ubiquitinated protein levels in the spinal cord of XBP-1^{WT} and XBP-1^{Nes-/-} mice, a slight increase in the amount of high-molecular weight ubiquitinated proteins in XBP-1^{Nes-/-} animals was observed. Similarly, a further increase was detected in SOD1^{G86R} mice bred on an XBP-1 deficient genetic background compared with control SOD1^{G86R} mice (FIG. 5E). In agreement with this finding, targeting of EDEM in NSC34 cells leads to a similar phenotype as shXBP-1 cells, increasing the accumulation of SOD1^{G86R} detergent insoluble species (FIG. 5F) and LC3-labeled autophagosomes (FIG. 5G).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

We claim:

1. A method of identifying compounds useful in treating or preventing a neurodegenerative disease associated with aberrant protein aggregation comprising,
a) providing an indicator cell comprising a polypeptide selected from the group consisting of XBP-1, IRE-1, and EDEM polypeptide;
b) contacting the indicator cell with each member of a library of test compounds;
c) determining the activity of the polypeptide in the presence and absence of the test compound;
d) selecting from the library of test compounds a compound of interest that downmodulates the activity of the polypeptide;
e) determining the effect of the compound on autophagy; and
f) selecting from the library of test compounds a compound of interest that increases autophagy as compared to an appropriate control, thereby identifying a compound useful in treating a neurodegenerative disease associated with aberrant protein aggregation.

2. The method of any one of claims 1, wherein the activity of the XBP-1, IRE-1 alpha, or EDEM polypeptide is determined by determining the level of XBP-1, IRE-1 alpha, or EDEM protein levels.

3. The method of any one of claims 1, wherein the binding of XBP-1 to IRE-1.

4. The method of any one of claims 1, wherein the kinase activity of IRE-1 is measured.

5. The method of claim 1, wherein the endonuclease activity of IRE-1 is measured.

6. The method of claim 1, further comprising determining the effect of the identified compound on the ratio of unspliced XBP-1 to spliced XBP-1 mRNA and/or protein.

7. The method of claim 1, wherein the activity of the EDEM polypeptide is determined by determining the binding of EDEM to calnexin.

8. The method of claim 1, wherein the activity of the EDEM polypeptide is determined by determining the ubiquitinated cellular protein levels

9. The method of claim 1, wherein the effect of the compound on autophagy is determined by determining the effect on the compound on motor neuron survival.

10. The method of claim 1, wherein the effect of the compound on autophagy is determined by determining the effect on the compound on generation of protein aggregation associated with the neurodegenerative disease.

11. The method of any one of claims 1, wherein the effect of the compound on autophagy is determined by determining the effect on the compound on apoptosis of the cell.

12. The method of claim 1, wherein the cell further comprises an expression vector comprising a nucleic acid molecule encoding a SOD1 polypeptide, and the effect of the compound on autophagy is determined by determining the effect on the compound on intracellular accumulation of SOD1 inclusions and/or the detergent insolubility of SOD1.

13. The method of claim 12, wherein the SOD1 polypeptide comprises SOD^{G93A} or SOD^{G86R}.

14. The method of claim 12, wherein the expression vector further comprises a nucleic acid molecule encoding a heterologous polypeptide.

15. The method of claim 1, wherein the indicator cell further comprises an expression vector comprising a nucleic acid molecule encoding an LC3, ds RED or EGFP polypeptide.

16. The method of claim 1, wherein the indicator cell further comprises an expression vector comprising a nucleic acid molecule encoding polyglutamine, and the effect of the compound on autophagy is determined by determining the effect on the compound on intracellular accumulation of polyglutamine inclusions and/or the detergent insolubility of polyglutamine.

17. The method of claim 1, wherein the cell has been engineered to express the polypeptide by introducing into the cell an expression vector encoding the polypeptide.

18. The method of claim 17, wherein the indicator cell is a motor neuron cell.

19. The method of claim 1, wherein the indicator cell is contacted with an agent that induces ER stress.

20. The method of claim 19, wherein the agent is tunicamycin or thapsigargin.

21. The method of claim 1, wherein the indicator cell is contacted with an inhibitor of autophagy.

22. The method of claim 21, wherein the inhibitor is 3-methyladenine or Wortmannin.

23. The method of claim 1, wherein the indicator cell is undergoing nutrient starvation.

24. The method of claim 1, further comprising determining the effect of the compound on the number of autophagosomes.

25. The method of claim 1, further comprising determining the effect of the compound on lysosome content.

26. The method of claim 1, further comprising determining the effect of the compound on Beclin-1 expression.

27. The method of claim 1, further comprising determining the effect of the compound on expression and/or processing of LC3.

28. The method of claim 1, further comprising determining the expression of a gene responsive to the activity of the polypeptide.

29. The method of claim 28, wherein the gene is selected from the group consisting of XBP-1, IRE-1 alpha, EDEM, ERdj4, Gpr58, Sec61, Wfs-1, Herp, Chop.

30. The method of claim 1, further comprising determining the effect of the identified compound on the effect of the compound on a neurodegenerative disease associated with protein aggregation in a non-human animal, comprising administering the test compound to the animal and determining the effect of test compound on disease onset, disease progression, and/or disease severity in the presence and absence of the test compound, to thereby identify a compound that modulates a neurodegenerative disease associated with protein aggregation.

31. A method for decreasing protein aggregation in a motor neuron cell, comprising contacting the motor neuron cell with an agent that decreases the activity of molecule selected from the group consisting of XBP-1, IRE-1 and EDEM, thereby decreasing protein aggregation in a motor neuron cell.

32. A method for treating a subject with a neurodegenerative disease associated with protein aggregation, comprising administering to the subject having a neurodegenerative disease an agent that decreases the activity of a molecule selected from the group consisting of XBP-1, IRE-1 and EDEM, thereby treating a neurodegenerative disease associated with protein aggregation in the subject.

33. The method of claim 32, wherein the neurodegenerative disease associated with protein aggregation is Amyotrophic lateral sclerosis.

34. The method of claim 32, wherein the neurodegenerative disease associated with protein aggregation is Huntington's disease.

35. The method of claim 31 or 32, wherein the agent is a chemical chaperone or an autophagy activator.

36. The method of claim 35, wherein the chemical chaperone is selected from the group consisting of glycerol, D20, dimethylsulfoxide (DMSO), 4-phenyl butyrate (PBA), tauroursodeoxycholic acid (TUDCA), ursodeoxycholic acid (UDCA), glycine betaine (betaine), glycerolphosphocholine (GPC), methylamines, and trimethylamine N-oxide (TMAO).

37. The method of claim 35, wherein the autophagy activator is selected from the group consisting of a proteasome inhibitor, rapamycin, analogues, and derivatives thereof, tamoxifen, IFN-gamma, trehalose and vinblastine.

38. A method for determining the predisposition of a subject to develop a neurodegenerative disorder associated with aberrant protein aggegation, said method comprising determining the amount of one or more markers selected from the group consisting of XBP-1, ERdj4, EDEM, WFS1, Grp58, and PDI in a biological sample derived from the nervous system of the subject and comparing the amount in said sample to the activity of the marker in an appropriate control sample, wherein an increase in the amount of the marker in the sample relative to the amount of the marker in the control sample indicates that the subject is at risk of developing neurodegenerative disorder associated with aberrant protein aggegation.
